(19) United States
(12) Patent Application Publication
(10) Pub. No.: US 2026/0199396 A1
(43) Pub. Date: Jul. 16, 2026

(54) METHOD OF TREATING HEART FAILURE IN SUBJECTS WITH PERSISTENT INFLAMMATION

(71) Applicant: MESOBLAST INTERNATIONAL SARL, Meyrin (CH)

(72) Inventors: Silviu ITESCU, Melbourne (AU); Paul SIMMONS, Melbourne (AU); Jack HAYES, New York, NY (US); Justin HORST, New York, NY (US); Fiona SEE, Melbourne (AU); Kenneth BOROW, New York, NY (US)

(73) Assignee: MESOBLAST INTERNATIONAL SARL, Meyrin (CH)

(21) Appl. No.: 19/136,677

(22) PCT Filed: Dec. 8, 2023
(86) PCT No.: PCT/IB2023/062427

§ 371 Date: Jun. 6, 2025

Related U.S. Application Data
(60) Provisional application No. 63/386,876, filed on Dec. 9, 2022.
(60) Provisional application No. 63/386,878, filed on Dec. 9, 2022.
(60) Provisional application No. 63/486,792, filed on Feb. 24, 2023.
(60) Provisional application No. 63/507,009, filed on Jun. 8, 2023.
(60) Provisional application No. 63/507,013, filed on Jun. 8, 2023.
(60) Provisional application No. 63/513,777, filed on Jul. 14, 2023.

(51) Int. Cl. A61K 35/28; A61P 9/04; C12N 5/00; C12N 5/0775; G01N 33/68

(52) U.S. Cl. CPC A61K 35/28; A61P 9/04; C12N 5/0663; C12N 5/562; G01N 33/68; G01N 33/6863; C12N 2500/98; C12N 2501/2306; C12N 2501/2308; C12N 2501/2317; C12N 2501/24; C12N 2501/25; G01N 2333/475; G01N 2333/515; G01N 2333/521

(57) ABSTRACT

The present disclosure relates to cellular compositions with anti-inflammatory properties and use of the same in methods for treating and/or preventing progressive heart failure.

### TECHNICAL FIELD

The present disclosure relates to cellular compositions with anti-inflammatory properties and use of the same in methods for treating and/or preventing progressive heart failure.

### BACKGROUND

Myocardial infarction (MI) is still one of the main causes of mortality and morbidity in developed countries. An update of US Medicare records was published that evaluated data involving 350,509 acute MI hospitalization in patients >65 years who were discharged alive after their event (Schuster et al. (2004) Physiol Heart Circa Physiol., 287(2):525-32). Within the first year post the index event, 25.9% of the MI patients died with 50.5% re-hospitalized. In the month after a MI, the likelihood of death was 21 times higher and the likelihood of hospitalization and was 12 times higher than among the general Medicare-age population.

During the past decade, numerous clinical trials evaluating novel drug therapies have been conducted in patients with advanced heart failure (HF). Despite progress made in reducing morbidity and mortality in patients with HF, those with advanced disease continue to experience an unfavourable clinical course characterized by frequent hospitalizations and premature death.

Clearly, there is a need in the art for treating or preventing progressive heart failure.

### SUMMARY

The present inventors have surprisingly identified that mesenchymal lineage precursor or stem cells (MLPSCs) which have been cultured in a culture media comprising non-fetal serum are particularly effective in treating certain subjects with progressive heart failure, in particular in the context of subjects with persistent inflammation. One reason this is surprising is because newborn calf serum (NBCS; a non-fetal serum) is generally marketed as an equivalent/acceptable substitute for fetal bovine serum (FBS). Indeed, the present inventors unexpectedly found that this was not the case, as NBCS supplementation improved therapeutic outcomes in patients. Analysis of the newborn serum used to culture MLPSCs with increased therapeutic efficacy surprisingly revealed increased levels of cytokines, in particular, in regard to cytokines where a corresponding receptor is expressed by MLPSCs. These findings underpin the use of novel MLPSC compositions through culture expansion with certain pro-inflammatory cytokines and/or in newborn serum.

Accordingly, in an example, the present disclosure relates to a method of treating progressive heart failure in a subject, the method comprising administering to the subject a composition comprising a population of culture expanded mesenchymal lineage precursor or stem cells (MLPSCs) or conditioned media obtained therefrom, wherein the subject has persistent inflammation and, wherein the MLPSCs have been culture expanded in a cell culture media comprising at least one pro-inflammatory cytokine. In an example, the pro-inflammatory cytokine is selected from the group consisting of IL-1B, IL-6, TNF-α, IFN-γ and/or IL-1RA. In an example, the MLPSCs have been culture expanded in media containing: IFN-gamma and/or TNF-alpha; and/or, one or more pro-inflammatory cytokines selected from the group consisting of IL-6; IL-8; IL-17A; MCP-1; MIP-1-alpha; MIP-1-beta; IP-10. In an example, the media contains three or more pro-inflammatory cytokines. In an example, the media contains two or more pro-inflammatory cytokines selected from the group consisting of IL-6; IL-8; IL-17A; MCP-1; MIP-1-alpha; MIP-1-beta; IP-10. In an example, the media contains IL-6. In an example, the media contains IL-8 and/or IL-17A. In an example, the media contains IFN-gamma and TNF-alpha. In an example, the media contains IFN-gamma. In an example, the level of IFN-gamma is <1 ng/ml. In an example, the level of IFN-gamma is <500 pg/ml. In an example, the level of IFN-gamma is <100 pg/ml. In an example, the media contains TNF-alpha. In an example, the level of TNF-alpha is <1 ng/ml. In an example, the level of TNF-alpha is <750 pg/ml. In an example, the level of TNF-alpha is <400 pg/ml.

In an example, the pro-inflammatory cytokine is provided in a non-fetal serum. Therefore, in an example, the cell culture media comprises non-fetal serum. In an example, the media contains serum which comprises the pro-inflammatory cytokines. In an example, the media comprises a non-fetal serum. In an example, the serum is a newborn mammalian serum. In an example, the serum is newborn calf serum (NBCS). In an example, the non-fetal serum is NBCS. In an example, the serum is obtained no more than 21 days after birth. In an example, the serum is obtained between the day of birth and 21 days after birth. In an example, the serum is obtained between the day of birth and 14 days after birth. In an example, the serum is obtained between the day of birth and 10 days after birth. In an example, the serum is obtained between the day of birth and 7 days after birth. In an example the media comprises at least 5% (v/v) newborn calf serum (NBCS). In an example, the media comprises 5% non-fetal serum. In an example, the media comprises 5% non-fetal serum and 5% fetal serum. In these examples, the non-fetal serum is NBCS. In an example, the fetal serum is fetal calf serum.

In an example, the media is serum free and/or xeno free. In an example, the media is a xeno-free media. In an example, the xeno-free media comprises human serum. In an example, the media is serum-free.

In an example, the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In another example, the present disclosure relates to a method of treating progressive heart failure in a subject, the method comprising administering to the subject a composition comprising a population of culture expanded mesenchymal lineage precursor or stem cells (MLPSCs), wherein the MLPSCs have been culture expanded in media containing Interferon (IFN)-gamma and/or tumor necrosis factor (TNF)-alpha, wherein the level(s) of IFN-gamma and/or TNF-alpha in the media are <1 ng/ml. For example, the level of IFN-gamma may be <500 pg/ml. In an example, the level of IFN-gamma is <100 pg/ml. In an example, the level of TNF-alpha is <750 pg/ml. In another example, the level of TNF-alpha is <500 pg/ml. In an example, the levels of IFN-gamma and TNF-alpha are both <500 pg/ml.

In an example, the administered composition comprises a culture-expanded population of mesenchymal lineage precursor or stem cells (MLPSCs), wherein the MLPSCs have been culture expanded in media containing one or more pro-inflammatory cytokines selected from the group consisting of IL-6; IL-8; IL-17A; MCP-1; MIP-1-alpha; MIP-1-beta; IP-10.

Accordingly, in another aspect, the administered composition comprises a culture-expanded population of mesenchymal lineage precursor or stem cells (MLPSCs), wherein the MLPSCs have been culture expanded in media containing:
IFN-gamma and/or TNF-alpha; and/or,
one or more pro-inflammatory cytokines selected from the group consisting of IL-6; IL-8; IL-17A; MCP-1; MIP-1-alpha; MIP-1-beta; IP-10. In an example, the media contains three or more pro-inflammatory cytokines. In another example, the media contains two or more pro-inflammatory cytokines selected from the group consisting of IL-6; IL-8; IL-17A; MCP-1; MIP-1-alpha; MIP-1-beta; IP-10. In another example, the media contains IL-6. In another example, the media contains IL-8 and/or IL-17A. In another example, the media contains IFN-gamma and TNF-alpha. For example, the media may contain IFN-gamma, TNF-alpha and, one or more pro-inflammatory cytokines selected from the group consisting of IL-6; IL-8; IL-17A; MCP-1; MIP-1-alpha; MIP-1-beta; IP-10.

In an example, the level of IFN-gamma in the media is <1 ng/ml. For example, the level of IFN-gamma may be <500 pg/ml. In an example, the level of IFN-gamma is <100 pg/ml. In another example, the level of TNF-alpha in the media is <1 ng/ml. For example, the level of TNF-alpha may be <750 pg/ml. In an example, the level of TNF-alpha is <400 pg/ml.

In an example, the media contains serum which comprises the pro-inflammatory cytokines. In an example, the serum is a non-fetal serum. In an example, the serum is a newborn mammalian serum. For example, the serum may be newborn calf serum. In an example, the newborn serum is obtained no more than 21 days after birth.

In an example, the media is characterised by one or more or all of the following:
a level of IFN-gamma greater than 1 pg/ml;
a level of TNF-alpha greater than 2 pg/ml;
a level of IL-6 greater than 3 pg/ml;
a level of IL-8 greater than 500 pg/ml;
a level of IL-17A greater than 0.2 pg/ml;
a level of MCP-1 greater than 3 pg/ml;
a level of MIP-1-alpha greater than 0.5 pg/ml;
a level of MIP-1-beta greater than 3 pg/ml;
a level of IP-10 greater than 500 pg/ml.

In an example, the media is characterised by one or more or all of the following:
a level of IFN-gamma between 1 pg/ml and <1 ng/ml;
a level of TNF-alpha between than 2 pg/ml and <1 ng/ml;
a level of IL-6 greater than 3 pg/ml;
a level of IL-8 greater than 500 pg/ml;
a level of IL-17A greater than 0.2 pg/ml;
a level of MCP-1 greater than 3 pg/ml;
a level of MIP-1-alpha greater than 0.5 pg/ml;
a level of MIP-1-beta greater than 3 pg/ml;
a level of IP-10 greater than 500 pg/ml.

In another example, the media is characterised by a level of IFN-gamma between 1 pg/ml and <1 ng/ml; a level of TNF-alpha between than 2 pg/ml and <1 ng/ml; and, one or more or all of the following:
a level of IFN-gamma between 1 pg/ml and <1 ng/ml;
a level of TNF-alpha between than 2 pg/ml and <1 ng/ml;
a level of IL-6 greater than 3 pg/ml;
a level of IL-8 greater than 500 pg/ml;
a level of IL-17A greater than 0.2 pg/ml;
a level of MCP-1 greater than 3 pg/ml;
a level of MIP-1-alpha greater than 0.5 pg/ml;
a level of MIP-1-beta greater than 3 pg/ml;
a level of IP-10 greater than 500 pg/ml

In another example, the media is characterised by supplementation with serum comprising one or more or all of the following:
a level of IFN-gamma greater than 10 pg/ml;
a level of TNF-alpha greater than 20 pg/ml;
a level of IL-6 greater than 30 pg/ml;
a level of IL-8 greater than 5,000 pg/ml;
a level of IL-17A greater than 2 pg/ml;
a level of MCP-1 greater than 30 pg/ml;
a level of MIP-1-alpha greater than 5 pg/ml;
a level of MIP-1-beta greater than 30 pg/ml;
a level of IP-10 greater than 5,000 pg/ml.

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In an example, the media comprises at least 5% (v/v) newborn mammalian serum. In another example, the media comprises 5% (v/v) newborn calf serum. In another example, the media is serum free.

In another example, the present disclosure relates to a method of treating progressive heart failure in a subject, the method comprising administering to the subject a composition comprising a population of culture expanded MLPSCs, wherein the MLPSCs have been culture expanded in a cell culture media comprising non-fetal serum.

In an example, the non-fetal serum is new born calf serum (NBCS). In an example, NBCS is obtained ≤21 days after birth of the calf. For example, the NBCS is obtained between the day of birth and 21 days after birth of the calf. In another example, the NBCS is obtained between the day of birth and 14 days after birth of the calf. In another example, the NBCS is obtained between the day of birth and 10 days after birth of the calf. In another example, the NBCS is obtained between the day of birth and 7 days after birth of the calf. In an example, the NBCS is obtained after the calf has received colostrum.

In an example, subjects treated according to the present disclosure have persistent inflammation.

In an example, persistent inflammation is characterised by elevated C-reactive protein (CRP). In an example, elevated CRP is characterised by CRP ≥2 mg/L. Accordingly, in an example, the subject's CRP level is ≥2 mg/L. In another example, the subject's CRP level is between 2 and 5 mg/L, preferably between 2 and 4 mg/L, more preferably between 2 and 3 mg/L.

In an example, the subject has persistent left ventricular dysfunction. In an example, the subject has a LVEF of less than about 45%. In an example, the subject has a LVEF of less than 40%. %. In an example, the subject has a LVEF of between 30 and 35%. In an example, the subject has a LVEF of between 30 and 35% or lower. In an example, the subject's LVEF is less than 35%. In another example, the subject has a LVESV greater than 70 ml. In an example, subject has a LVESV between 70 ml and 160 ml.

In an example, the subject has Class II heart failure according to the New York Heart Association (NYHA) classification scale. In an example, the subject has myocardial ischemia and/or diabetes. In an example, the subject's level of N-terminal pro-B-type natriuretic peptide (NT-proBNP) is: >1000 pg/mL, or, between 1000 pg/ml and 2500 pg/ml. In an example, the subject has had a heart failure hospitalisation event over the previous 9 months. In an example, the subject's heart failure results from an ischaemic event or from a non-ischaemic event. In an example, the heart failure results from an ischemic event.

In an example, methods of the disclosure comprise the steps of: i) selecting a subject having progressive heart failure for treatment, wherein the subject has a micro-vascular disease and/or a macro-vascular disease, and ii) administering the MLPSCs. For example, the method comprises the steps of: i) selecting a subject having a micro-vascular disease and/or a macro-vascular disease for treatment, and ii) administering the MLPSCs. In an embodiment of these examples, the subject has persistent inflammation.

In an example, the method comprises the steps of: i) selecting a subject having a CRP level ≥2 mg/L for treatment, and ii) administering to the MLPSCs.

In an example, methods of the disclosure comprise the steps of: i) selecting a subject having progressive heart failure and a CRP level ≥2 mg/L for treatment, and ii) administering the MLPSCs. In an example, the subject's CRP level is between 2 and 5 mg/L. In an example, the subject's CRP level is between 2 and 4 mg/L. In an example, the subject's CRP level is between 2 and 3 mg/L.

In an example, the composition is administered transendocardially and/or intravenously. In an example, methods of treatment disclosed herein comprise administering between 1×10⁷ and 2×10⁸ cells.

In an example, the subject has a reduced risk of cardiac death after treatment. In an example, the reduced risk is relative to risk of cardiac death in a subject that has not been administered MLPSCs.

In an example, treatment improves the subject's LVEF by at least 4 percentage points. In an example, treatment improves the subject's LVEF by at least 5 percentage points or at least 6 percentage points. In an example, treatment improves the subject's LVEF by between 4 and 7 percentage points. In an example, treatment improves the subject's LVEF by between 5 and 7 percentage points.

In an example, treatment improves the subject's L VESV by at least 17 ml. In an example, treatment improves the subject's LVESV by at least 20 ml. In an example, treatment improves the subject's LVESV by between 15 ml and 30 ml.

In an example, treatment improves the subject's LVEDV by at least 15 ml. In an example, treatment improves the subject's LVEDV by between 15 ml and 25 ml.

In an example, the subject has a reduced risk of ischaemic MACE (MI or stroke) after treatment.

In an example, the subject has a left ventricular assist device (LVAD). In an example, the subject has a LVAD and heart failure resulting from an ischemic event. In an example, the subject's IL-6 level is increased relative to baseline 60 days after LVAD implantation. In an example, treatment reduces the subject's risk of all-cause death. In an example, treatment reduces the subject's risk of all-cause death by between 10% and 90%. In an example, treatment reduces the subject's risk of all-cause death by greater than 50%. In an example, treatment reduces the subject's risk of all-cause death by between 20 and 85%. In an example, treatment reduces the subject's risk of all-cause death by about 80%. In an example, the reduced risk is relative to risk of all-cause death in a subject that has not been administered MLPSCs.

In an example, the MLPSCs are mesenchymal precursor cells (MPCs). In an example, the MPCs are isolated from bone mononuclear cells with an anti-STRO-3 antibody before culture expansion.

In an example, the MLPSCs are mesenchymal stem cells (MSCs).

In an example, the MLPSCs are allogeneic.

In an example, the cells have been cryopreserved prior to administration.

In an example, populations of MLPSC disclosed herein are administered in a composition. In an example, the composition further comprises Plasma-Lyte A, dimethyl sulfoxide (DMSO), human serum albumin (HSA). In another example, the composition further comprises Plasma-Lyte A (70%), DMSO (10%), HSA (25%) solution, the HSA solution comprising 5% HSA and 15% buffer.

In an example, the composition comprises greater than 6.68×10⁶ viable cells/mL.

In an example, the composition comprises human bone marrow-derived allogeneic MPCs isolated from bone mononuclear cells with anti-STRO-3 antibodies, expanded ex vivo in culture media comprising NBCS, and cryopreserved.

The present inventors have also surprisingly identified that MLPSCs which have been cultured in a culture media comprising certain pro-inflammatory cytokines and/or a non-fetal serum increase angiogenesis and express increased levels of angiogenic markers. Thus, the present inventors have arrived at criteria which can be used in one or more informative potency assay(s) to establish therapeutic efficacy of culture expanded MLPSC populations (or conditioned media obtained therefrom), in particular in the context of progressive heart failure.

Accordingly, in an example, the present disclosure provides a method for selecting a population of culture expanded MLPSCs for use in treatment of progressive heart failure in a subject wherein the MLPSCs have been culture expanded in a cell culture media comprising at least one pro-inflammatory cytokine, the method comprising: (i) obtaining a population of MLPSCs, (ii) determining the level of one or more angiogenic markers in the population of MLPSCs, wherein the one or more angiogenic marker(s) is selected from the group consisting of: the level of VEGF, angiogenin, SDF-1a expressed by the MLPSCs under culture conditions; and/or, the level of endothelial network formation, endothelial network length, endothelial branch length measured after treating a population of endothelial cells with conditioned media obtained from the MLPSCs; (iii) selecting for use in treatment the MLPSCs that have increased level(s) of the one or more angiogenic markers. In an example, the treatment is treatment of progressive heart failure. In an example, MLPSCs are selected for use in treatment based on:
an increased level of one or more of VEGF, angiogenin, SDF-1a expressed by the MLPSCs under culture conditions; and,
an increased level of one or more of endothelial network formation, endothelial network length, endothelial branch length measured after treating a population of endothelial cells with conditioned media obtained from the MLPSCs.

In an example, MLPSC conditioned media or soluble factors derived therefrom are selected for use in treatment based on:
an increased level of one or more of VEGF, angiogenin, SDF-1a expressed by the MLPSCs under culture conditions; and,
an increased level of one or more of endothelial network formation, endothelial network length, endothelial branch length measured after treating a population of endothelial cells with conditioned media obtained from the MLPSCs.

In an example, the soluble factors derived from conditioned media are exosomes.

In an embodiment of the above referenced example, the pro-inflammatory cytokine(s) is one or more of the above referenced cytokine(s) or combinations thereof.

In an embodiment of the above referenced example, the pro-inflammatory cytokine(s) is provided in a non-fetal serum. Accordingly, in an example, the cell culture media comprises a non-fetal serum such as new born calf serum.

In another example, the present disclosure provides a method for determining the potency of a population of culture expanded MLPSCs or conditioned media obtained therefore, wherein the MLPSCs have been culture expanded in a cell culture media comprising at least one pro-inflammatory cytokine, the method comprising determining the level of one or more angiogenic markers in the population of MLPSCs, wherein the one or more angiogenic markers is selected from the group consisting of: the level of VEGF, angiogenin, SDF-1a expressed by the MLPSCs under culture conditions; and/or, the level of endothelial network formation, endothelial network length, endothelial branch length measured after treating a population of endothelial cells with conditioned media obtained from the MLPSCs, wherein an increased level of one or more angiogenic markers is indicative of biological activity or therapeutic efficacy. In an example, an increased level of one or more angiogenic markers is indicative of biological activity or therapeutic efficacy in progressive heart failure.

In an embodiment of the above referenced example, the pro-inflammatory cytokine is provided in a non-fetal serum. Accordingly, in an example, the cell culture media comprises a non-fetal serum such as new born calf serum.

In an example, the increased level of the one or more angiogenic markers is determined relative to a control population of MLPSCs. For example, an increased level of one or more angiogenic markers may be determined relative to a population of MLPSCs that have been culture expanded in a cell culture media comprising 10% fetal calf serum. In an example, an appropriate control does not contain a non-fetal serum.

In an example, the population of MLPSCs used in the methods of the present disclosure have been cultured expanded in culture media comprising a non-fetal serum, cryopreserved and thawed. In an example, potency is assessed after the MLPSCs have been cultured expanded, cryopreserved and thawed. In another example, potency is assessed after the MLPSCs have been cultured expanded, cryopreserved and thawed twice.

In an example, the level of VEGF is greater than about 3 ng/ml, preferably greater than about 3.45 ng/mL.

In an example, the level of angiogenin is greater than about 1000 pg/ml, preferably greater than about 1114 pg/ml.

In an example, the level of SDF-1a is greater than about 3000 ng/ml.

In an example, the endothelial network formation is greater than about 0.1 mm²/mm², preferably greater than about 0.12 mm²/mm².

In an example, the endothelial network length is greater than about 4 mm²/mm², preferably greater than about 5 mm²/mm².

In an example, the endothelial branch length is greater than about 12 l/mm², preferably about 15 l/mm².

In an example, endothelial network formation, endothelial network length, and/or endothelial branch length are measured using an in-vitro angiogenesis assay.

In an example, the method further comprises culture expanding a selected MLPSC population to provide a pharmaceutical composition.

In an example, the disclosure provides a selected population of MLPSCs obtained by the methods disclosed herein. In an example, the disclosure provides a cryopreserved cellular intermediate comprising a population of culture expanded MLPSCs that have been selected according to the method disclosed herein. In an example, the selected population of MLPSCs is for use in a method of treating progressive heart failure.

The present inventors have also surprisingly identified a novel population of MLPSCs characterised as having high angiogenic potential, as determined by the level of expression of one or more angiogenic markers.

Accordingly, in an example, the disclosure provides a culture expanded population of mesenchymal lineage precursor or stem cells (MLPSCs), wherein the population of MLPSCs are selected based on high angiogenic potential as determined by the level of angiogenin expressed by the MLPSCs under culture conditions. In an example, the MLPSCs have been culture expanded in a cell culture media comprising at least one pro-inflammatory cytokine as disclosed herein.

In an example, MLPSCs that express an increased level of angiogenin relative to a control population are selected. In an example, the control population is a population of MLPSCs that have been culture expanded in a cell culture media comprising 10% fetal calf serum. In an example, MLPSCs that express a level of angiogenin greater than about 1200 pg/ml are selected. In an example, MLPSCs that express a level of angiogenin greater than about 1100 pg/ml are selected. In an example, MLPSCs that express a level of angiogenin greater than about 1000 pg/ml are selected. In an example, MLPSCs that express a level of angiogenin greater than about 700 pg/ml are selected. In an example, the level of angiogenin is measured in conditioned media obtained from the culture expanded cells. In an example, the level of angiogenin is measured in cell lysates of culture expanded cells.

In an example, the disclosure provides a culture expanded population of mesenchymal lineage precursor or stem cells (MLPSCs), wherein the population of MLPSCs are selected based on high angiogenic potential as determined by the level of one or more of the following measured after treating a population of endothelial cells with conditioned media obtained from the MLPSCs:
endothelial network formation;
endothelial network length; or,
endothelial branch length.

In an example, the MLPSCs have been culture expanded in a cell culture media comprising at least one pro-inflammatory cytokine as disclosed herein.

In an example, MLPSCs that induce increased levels of one or more of endothelial network formation, endothelial length, or endothelial branch length, relative to a control population are selected. In an example, the control population is a population of MLPSCs that have been culture expanded in a cell culture media comprising 10% fetal calf serum.

In an example, MLPSCs that induce endothelial network formation greater than about 0.12 mm²/mm² are selected. In an example, MLPSCs that induce endothelial network formation greater than about 0.11 mm²/mm² are selected. In an example, MLPSCs that induce endothelial network formation greater than about 0.10 mm²/mm² are selected. In an example, MLPSCs that induce endothelial network formation greater than about 0.14 mm²/mm² are selected.

In an example, MLPSCs that induce endothelial network length greater than about 5 mm²/mm² are selected. In an example, MLPSCs that induce endothelial network length greater than about 4 mm²/mm² are selected. In an example, MLPSCs that induce endothelial network length greater than about 5.5 mm²/mm² are selected. In an example, MLPSCs that induce endothelial network length greater than about 5.75 mm²/mm² are selected.

In an example, MLPSCs that induce endothelial branch length greater than about 15 l/mm² are selected. In an example, MLPSCs that induce endothelial branch length greater than about 14 l/mm² are selected. In an example, MLPSCs that induce endothelial branch length greater than about 10 l/mm² are selected. In an example, MLPSCs that induce endothelial branch length greater than about 16 l/mm² are selected.

The present disclosure also provides a method of manufacturing drug product which comprises a population of mesenchymal lineage precursor or stem cells (MLPSCs), the method comprising: acquiring a determination of whether a test population of MLPSCs have a predetermined level of angiogenic potential under culture conditions, and processing at least a portion of the test population of MLPSCs as a drug product if the test population of MLPSCs have at least the predetermined level of angiogenic potential under culture conditions, thereby manufacturing the drug product; or discarding at least a portion of the test population of MLPSCs if the population of MLPSCs has less than the predetermined level of angiogenic potential under culture conditions, wherein angiogenic potential is measured by a predetermined level of angiogenin measured under culture conditions.

In an example, the predetermined level of angiogenin is an increase relative to a control population. For example, the control population is a population of MLPSCs that have been culture expanded in a cell culture media comprising 10% fetal calf serum. In an example, the predetermined level of angiogenin is greater than about 1200 pg/ml. In an example, the predetermined level of angiogenin is greater than about 1100 pg/ml. In an example, the predetermined level of angiogenin is greater than about 1000 pg/ml. In an example, the predetermined level of angiogenin is greater than about 700 pg/ml. In an example, the predetermined level of angiogenin is measured in conditioned media obtained from the test population of MLPSCs. In an example, the predetermined level of angiogenin is measured in conditioned media obtained from the culture expanded cells. In an example, the predetermined level of angiogenin is measured in lysates of culture expanded cells.

In an example, the disclosure provides a method of manufacturing drug product which comprises a population of mesenchymal lineage precursor or stem cells (MLPSCs), the method comprising: acquiring a determination of whether a test population of MLPSCs have a predetermined level of angiogenic potential under culture conditions, and processing at least a portion of the test population of MLPSCs as a drug product if the test population of MLPSCs have at least the predetermined level of angiogenic potential under culture conditions, thereby manufacturing the drug product; or discarding at least a portion of the test population of MLPSCs if the population of MLPSCs has less than the predetermined level of angiogenic potential under culture conditions, wherein angiogenic potential is measured by:
a predetermined level of one or more of the following as measured after treating a population of endothelial cells with conditioned media obtained from the MLPSCs:
   endothelial network formation;
   endothelial network length; and/or,
   endothelial branch length measured in an in-vitro angiogenesis assay.

In an example, the predetermined level of one or more of endothelial network formation, endothelial length, or endothelial branch length is an increase relative to a control population. For example, the control population is a population of MLPSCs that have been culture expanded in a cell culture media comprising 10% fetal calf serum.

In an example, the predetermined level of endothelial network formation is greater than about 0.12 mm²/mm². In an example, the predetermined level of endothelial network formation is greater than about 0.11 mm²/mm². In an example, the predetermined level of endothelial network formation is greater than about 0.10 mm²/mm². In an example, the predetermined level of endothelial network formation is greater than about 0.14 mm²/mm².

In an example, the predetermined level of endothelial network length is greater than about 5 mm²/mm². In an example, the predetermined level of endothelial network length is greater than about 4 mm²/mm². In an example, the predetermined level of endothelial network length is greater than about 5.5 mm²/mm². In an example, the predetermined level of endothelial network length is greater than about 5.75 mm²/mm².

In an example, the predetermined level of endothelial branch length is greater than about 15 l/mm². In an example, the predetermined level of endothelial branch length is greater than about 14 l/mm². In an example, the predetermined level of endothelial branch length is greater than about 10 l/mm². In an example, the predetermined level of endothelial branch length is greater than about 16 l/mm².

In an example, MLPSCs with high angiogenic potential according to the disclosure can be culture expanded in a cell culture media which comprises at least one pro-inflammatory cytokine. In an example, the MLPSCs have been culture expanded in media containing: IFN-gamma and/or TNF-alpha; and/or, one or more pro-inflammatory cytokines selected from the group consisting of IL-6; IL-8; IL-17A; MCP-1; MIP-1-alpha; MIP-1-beta; IP-10. In an example, the media contains three or more pro-inflammatory cytokines. In an example, the media contains two or more pro-inflammatory cytokines selected from the group consisting of IL-6; IL-8; IL-17A; MCP-1; MIP-1-alpha; MIP-1-beta; IP-10. In an example, the media contains IL-6. In an example, the media contains IL-8 and/or IL-17A. In an example, the media contains IFN-gamma and TNF-alpha. In an example, the media contains IFN-gamma. In an example, the level of IFN-gamma is <1 ng/ml. In an example, the level of IFN-gamma is <500 pg/ml. In an example, the level of IFN-gamma is <100 pg/ml. In an example, the media contains TNF-alpha. In an example, the level of TNF-alpha is <1 ng/ml. In an example, the level of TNF-alpha is <750 pg/ml. In an example, the level of TNF-alpha is <400 pg/ml.

In an example, the pro-inflammatory cytokine is provided in a non-fetal serum. Therefore, in an example, the cell culture media comprises non-fetal serum. In an example, the media contains serum which comprises the pro-inflammatory cytokines. In an example, the media comprises a non-fetal serum. In an example, the serum is a newborn mammalian serum. In an example, the serum is newborn calf serum (NBCS). In an example, the non-fetal serum is NBCS. In an example, the serum is obtained no more than 21 days after birth. In an example, the serum is obtained between the day of birth and 21 days after birth. In an example, the serum is obtained between the day of birth and 14 days after birth. In an example, the serum is obtained between the day of birth and 10 days after birth. In an example, the serum is obtained between the day of birth and 7 days after birth. In an example the media comprises at least 5% (v/v) newborn calf serum (NBCS). In an example, the media comprises 5% non-fetal serum. In an example, the media comprises 5% non-fetal serum and 5% fetal serum. In these examples, the non-fetal serum is NBCS.

In an example, the media is serum free and/or xeno free. In an example, the media is a xeno-free media. In an example, the xeno-free media comprises human serum. In an example, the media is serum-free.

In an example, the pro-inflammatory cytokine is provided in a non-fetal serum. Accordingly, in an example, MLPSCs have been culture expanded in a culture media comprising a non-fetal serum. In an example, the non-fetal serum is a newborn serum. In an example, the non-fetal serum is a newborn mammalian serum. In an example, the newborn mammalian serum is newborn calf serum (NBCS). In an example, MLPSCs have been culture expanded in a culture media comprising about 5% non-fetal serum. In an example, MLPSCs have been culture expanded in a culture media comprising between about 5% non-fetal serum and about 10% non-fetal serum. In an example, MLPSCs have been culture expanded in a culture media comprising about 5% non-fetal serum and about 5% fetal serum. In these examples, non-fetal serum can be a newborn mammalian serum, for example newborn calf serum (NBCS). Accordingly, in an example, MLPSCs have been culture expanded in a culture media comprising between about 5% NBCS. In an example, MLPSCs have been culture expanded in a culture media comprising less than 10% fetal calf serum.

In an example, the pro-inflammatory cytokine is provided in xeno-free media. In an example, the pro-inflammatory cytokine is provided in serum free media. Accordingly, in an example, MLPSCs have been culture expanded in a culture media comprising xeno-free media. In an example, the xeno-free media comprises human serum. In an example, the xeno-free media comprises 3% human serum. In another example, the xeno-free media is serum-free. Accordingly, in an example, the MLPSCs have been culture expanded in a serum-free culture medium.

In an example, the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In an example, the level of angiogenin, endothelial network formation, endothelial network length, and/or endothelial branch length is indicative of biological activity or therapeutic efficacy of the culture expanded MLPSCs.

### BRIEF DESCRIPTION OF DRAWINGS

FIG. 1: Serum cytokine levels assessment and comparison. 1:1 FCS/NBCS (serum A); fetal bovine serum (serum B); FBS from a different supplier (serum C).

FIG. 2: Quantitative measurement of in-vitro angiogenesis induced by MLPSC conditioned media using IncuCyte® 96-Well Kinetic Angiogenesis PrimeKit Assay.

FIG. 3: Luminex assay results showing increased production of angiogenin by MLPSCs cultured in the presence or absence of newborn serum.

FIG. 4: Angiogenic marker levels in MLPSC-conditioned media from cGMP lots cultured in the presence or absence of newborn serum.

FIG. 5: Analysis of Change from Baseline at 12 Months in Echo Parameters—all subjects.

FIG. 6: Analysis of Change from Baseline at 12 Months in Echo Parameters—subjects with persistent inflammation (hsCRP ≥2).

FIG. 7: Analysis of Change from Baseline at 12 Months in Echo Parameters—subjects without persistent inflammation (hsCRP <2).

FIG. 8: MPCs cultured in media supplemented with newborn serum significantly reduced 3-point MACE in all patients.

FIG. 9: CV Death in in subjects with persistent inflammation (hsCRP ≥2) by MPCs cultured in the presence or absence of non-fetal serum.

FIG. 10: 3-Point composite MACE (MI, Stroke or CV Death) in subjects with persistent inflammation (hsCRP ≥2) by MPCs cultured in the presence or absence of non-fetal serum.

FIG. 11: MPCs cultured in media supplemented with newborn serum significantly reduced CV death (A) and TCE (B) in highest risk patients (CRP>2 mg/ml; NTpro-BNP>1000 ng/ml).

FIG. 12: (A) Pro-inflammatory cytokine IL-6 plasma levels in control LVAD patients: ischemic versus non-ischemic HFrEF etiology. (B) Pro-inflammatory cytokine IL-6 plasma levels in LVAD patients: ischemic controls versus ischemic LVAD patients administered MPCs.

FIG. 13: All-Cause Death over 12 months in ischemic and non-ischemic LVAD control patients.

FIG. 14: All-Cause Death over 12 months in LVAD patients administered “Licensed” Rexlemestrocel-L formulation, “Unlicensed” Rexlemestrocel-L formulation, and control patients. (A) All LVAD patients (ischemic and non-ischemic groups). (B) Ischemic LVAD patients.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

In an example, a sample is obtained from a patient or subject (e.g. a blood sample) and the level of a substance is measured in the sample. For example, a blood sample can be obtained to measure the level of CRP in a subject.

“C-reactive protein” or “CRP” is an inflammatory mediator. CRP levels are raised under conditions of acute inflammatory recurrence and rapidly normalize once the inflammation subsides. Accordingly, CRP is an effective marker of persistent inflammation. In an example, subjects treated according to the present disclosure may have elevated CRP. The term “elevated CRP” is used in the context of the present disclosure to refer to CRP levels that are increased relative to baseline CRP levels. In an example, CRP levels ≥1 mg/L are elevated. In another example, CRP levels ≥1.5 mg/L are elevated. In another example, CRP levels ≥2 mg/L are elevated. In an example, persistent inflammation is characterised by CRP levels ≥2 mg/L.

The term “level” is used to define the amount of a particular substance present in a sample, cell culture medium, serum preparation or compositions of the present disclosure. For example, a particular concentration, weight, percentage (e.g. v/v %) or ratio can be used to define the level of a particular substance.

The term “conditioned media” is used in the context of the present disclosure to refer to media obtained from MLPSCs under culture conditions. Such media contains the MLPSC secretome, proteins shed from the surface of MLPSCs and, other particles such as extracellular vesicles. Conditioned media of the disclosure contains pro-angiogenic factors such as extracellular vesicles, Angiogenin or secreted metabolites such as prostaglandin E2. The pro-angiogenic capabilities of conditioned media disclosed herein and/or factors obtained therefrom can be confirmed, if necessary, using one or more of the angiogenesis assays disclosed herein (e.g. endothelial network formation; endothelial length; endothelial branch length). In certain examples, the present disclosure relates to extracellular vesicles such as exosomes that have been obtained from conditioned media obtained from MLPSCs under culture conditions. In an example, the conditioned media is obtained when the MLPSCs are in exponential growth phase. In an example, the conditioned media is obtained after at least two or three days in culture. In another example, the conditioned media is obtained after about 30 to 84 hours of culture.

In an example, the level of a particular marker, such as a pro-angiogenic factor(s), is determined under culture conditions. The term “culture conditions” is used to refer to cells growing in culture. In an example, culture conditions refers to an actively dividing population of cells. Such cells may, in an example, be in exponential growth phase. Alternatively, such cells may be in a stationary phase.

In an example, in the context of measuring the level of IL2-RA inhibition, culture conditions can encompass co-culture of an MLPSC population disclosed herein and a second cell population such as a population which comprises peripheral blood mononuclear cells (PBMC). In an example, co-culture comprises culturing an MLPSC population disclosed herein and a population of activated PBMC. For example, PBMC can be activated using anti-CD3 and anti-CD28 antibodies before co-culture with an MLPSC population disclosed herein. In this example, “culture conditions” can comprise co-culturing MLPSCs and T cells at a ratio of about 1 MLPSC:2 T cells, or less. For example, 1:3, 1:4, 1:5, 1:10, 1:20, 1:30, 1:40, 1:50, 1:60, 1:70 1:80, 1:90, or 1 MLPSC:100 T cells, or less. In this example, the level of IL2-RA inhibition is determined after about 30 to 84 hours of cell culture under culture conditions.

In an example, the level is expressed in terms of how much of a particular marker is released from cells described herein under culture conditions.

In an example, the level is expressed in mg/L. For example, a level of CRP can be expressed in mg/L. In an example, the level is expressed in ng/ml. For example, a level of VEGF can be expressed in ng/ml. In an example, a level of SDF-1a can be expressed in ng/ml. In an example, the level is expressed in pg/ml. For example, a level of NT-proBNP can be expressed in pg/ml. In an example, a level of angiogenin can be expressed in pg/ml.

In an example, the level of a particular marker is measured in a population of cells (or supernatant obtained following cell culture of the same) and divided by the number of cells in the population. In this example, the level may be presented in units (e.g. pg) per 10⁶ cells.

In an example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art will appreciate that secreted markers can be measured by sampling the culture media while markers expressed on the surface of the cell may be measured by assessing a sample of cell lysate. In an example, the sample is taken when the cells are in exponential growth phase. In an example, the sample is taken after at least two or three days in culture. In another example, the sample is taken after about 30 to 84 hours of culture. In another example, the sample is taken when the cells are in a stationary phase.

In an example, the sample is taken from a co-culture of MLPSCs and activated PBMCs. In this example, the cell sample can be lysed and the level of a marker can be determined. For example, the level of IL2-RA may be determined. In his example, the level of IL2-RA can be determined using various methods such as an enzyme-linked immunosorbent assay (ELISA) based method. In an example, the ELISA comprises:
(i) adding sample diluent to each well of a microplate precoated with a monoclonal antibody specific for IL2-RA;
(ii) adding a co-cultured sample to a well of a microplate precoated with a monoclonal antibody specific for IL2-RA;
(iii) incubating the microplate for sufficient time to allow for the monoclonal antibody specific for IL2-RA to specifically bind to any IL2-RA in the sample;
(iv) washing the microplate;
(v) adding IL2-RA conjugate to the well;
(vi) incubating the microplate for sufficient time to allow the conjugate to specifically bind to any captured IL2-RA;
(vii) washing the microplate;
(viii) adding a substrate solution to the well;
(ix) incubating the microplate for sufficient time for colour development;
(x) adding a stop solution to the well;
(xi) reading optical density on a microplate reader set to 450 nm with wavelength correction at 570 nm;
(xii) determining the level of IL2-RA.

In another example, the level of IL2-RA is determined using fluorescence-activated cell sorting (FACS) using appropriate antibodies such as anti-CD25. Further antibodies may also be employed if required to distinguish CD25+ cell types. While the above referenced examples refer to IL-2RA, it will be appreciated that similar methods may also be used to determine the level of other markers disclosed herein such as angiogenin. In these examples, co-culture may not be required to determine the level. For example, the level of angiogenin may be measured in a population of MLPSCs under culture conditions.

In another example, the level is measured based on an assessment of conditioned media (or properties thereof) obtained from a population of MLPSCs under culture conditions. For example, conditioned media can be obtained from a population of MLPSCs disclosed herein under culture conditions before being used in one or more angiogenesis assays disclosed below

In an example, methods of manufacturing drug product according to the present disclosure comprise determining the level of one or more angiogenic markers under culture conditions.

Culture expanding cells from a cryopreserved intermediate means thawing cells subject to cryogenic freezing and in vitro culturing under conditions suitable for growth of the cells.

In an example, the “level” of a particular marker is determined after cells have been cryopreserved and then seeded back into culture. For example, the level may be determined after a first cryopreservation of cells. In another example, the level is determined after a second cryopreservation of cells. In an example, cells are isolated from an appropriate stem cell source such as bone marrow (e.g. using immune-selection for marker(s) such as STRO-1), culture expanded to provide an intermediate cell population and assessed to determine the level of a particular marker. In this example, the level may be determined before or after cryopreservation. In an example, the level is determined after cryopreservation of the intermediate cell population. In another example, cells may be culture expanded from a cryopreserved intermediate, cryopreserved a second time before being re-seeded in culture so that the level of a particular marker can be determined under culture conditions.

As used herein, the terms “treating”, “treat”, “treatment”, “reducing progression” include administering a population of mesenchymal lineage stem or precursor cells cultured according to the present disclosure and/or progeny thereof and/or soluble factors derived therefrom and/or extracellular vesicles derived therefrom to thereby reduce or eliminate at least one symptom of progressive heart failure or, in the context of reducing progression, delay development of the same.

In an example, the present disclosure encompasses selecting certain subjects with progressive heart failure for treatment with a cellular composition disclosed herein. In an example, subjects with persistent inflammation are selected for treatment. In an example, persistent inflammation is determined based on CRP level. For example, subjects with persistent inflammation have elevated CRP. In an example, subjects with CRP levels ≥2 mg/L are selected for treatment. In an example, persistent inflammation is determined based on IL-6 level. For example, subjects with persistent inflammation have elevated IL-6. In an example, subjects with persistently elevated IL-6 levels post-LVAD implantation are selected for treatment. In another example, subjects with micro-vascular disease and/or macro-vascular disease are selected for treatment. In an example, subjects with Class II heart failure are selected for treatment. In an example, subjects having elevated risk of cardiac death are selected for treatment.

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure. Subjects in need of treatment include those already having progressive heart failure as well as those in which progressive heart failure is to be prevented, delayed or halted.

In an example, compositions of the disclosure comprise genetically unmodified MLPSCs. As used herein, the term “genetically unmodified” refers to cells that have not been modified by transfection with a nucleic acid. For the avoidance of doubt, in the context of the present disclosure a MLPSC transfected with a nucleic acid encoding a protein would be considered genetically modified.

The term “angiogenic marker” as used herein refers an indicator of angiogenesis. As used herein, “angiogenic markers” include pro-angiogenic molecules, for example, VEGF, angiogenin, and SDF-1α. In another example, angiogenic markers are cellular indicators of angiogenesis, for example, endothelial network formation, endothelial network length, and endothelial branch length. In this example, cellular indicators of angiogenesis are determined in an in-vitro angiogenesis assay as disclosed herein. In an example, angiogenic marker characterisation may be used to characterise a MLPSC population disclosed herein (e.g. a cryopreserved intermediate or drug product disclosed herein).

The term “angiogenic potential” as used herein refers to the capability of an MLPSC population to express one or more angiogenic markers. In an example, angiogenic potential is determined by capacity to induce angiogenesis via an assay of the disclosure. In an example, MLPSCs of the disclosure have increased angiogenic potential. In another example, conditioned media of the disclosure has increased angiogenic potential. In an example, MLPSCs with increased angiogenic potential have been culture expanded according to the methods disclosed herein. For example, the MLPSCs can be culture expanded in media supplemented with pro-inflammatory cytokines disclosed herein and/or a non-fetal serum such as new born calf serum. In an example, MLPSCs or conditioned media of the disclosure have increased angiogenic potential relative to MLPSCs that have been culture expanded in media containing 10% FCS.

As used herein, the term “sample” refers to an extract from a subject or cell culture in which the level of a particular marker can be measured. The “sample” includes extracts and/or derivatives and/or fractions of the sample. In an example, the sample is an extract from a subject in which CRP levels can be measured. In the present disclosure, any biological material can be used as the above-mentioned sample so long as it can be collected from the subject or cell culture and assayed to determine the level of a marker disclosed herein (e.g. level of CRP in a subject). In an example, the sample is a blood sample. For example, the blood sample can be obtained from a subject with NYHA Class II heart failure.

In an example, the “sample” is a population of cells, for example a population of cells under culture conditions. In an example, the sample is supernatant obtained following cell culture, for example, cell conditioned media. In these examples, the sample is any extract of cell culture in which angiogenic markers can be measured. In an example, the sample is contacted with another cell population to determine the level of an angiogenic marker.

In an example, the present disclosure encompasses selecting a population of culture expanded MLPSCs of a certain potency for use in methods of treatment disclosed herein. The term “potency” as used herein refers to the specific ability or capacity of the MLPSCs to effect a given result. In an example, the result is a therapeutic result, for example an improvement in cardiac outcomes as disclosed herein.

“Therapeutic efficacy” is used in the context of the present disclosure to refer to MLPSCs and compositions disclosed herein that can treat, inhibit and/or prevent disease. For example, therapeutically effective MLPSCs and compositions disclosed herein can treat inhibit and/or prevent progressive heart failure.

“Biological activity” is used in the context of the present disclosure to define MLPSCs and compositions disclosed herein based on a particular activity. In an example, the biological activity is pro-angiogenic and/or anti-inflammatory activity. In an example, the biological activity is capacity to increase in-vitro angiogenesis. In an example, the biological activity is the increased expression of one or more angiogenic markers. In an example, the biological activity is angiogenic potential, as determined by the level of one or more angiogenic markers. In an example, biological activity is characterised by an improved clinical outcome(s) (e.g. survival) and/or parameter(s) (e.g. LVEF).

The term “clinically proven” (used independently or to modify the term “effective”) shall mean that efficacy has been proven by a clinical trial wherein the clinical trial has met the approval standards of U.S. Food and Drug Administration, EMEA or a corresponding national regulatory agency. For example, the clinical study may be an adequately sized, randomized, double-blinded study used to clinically prove the effects of the composition. In an example, a clinically proven effective amount is an amount shown by a clinical trial to meet a specified endpoint. In an example, the end point is protection against death. Put another way, the end point increases survival. For example, 100 day survival may be increased when administering treatment according to the present disclosure.

Accordingly, the terms “clinically proven efficacy” and “clinically proven effective” can be used in the context of the present disclosure to refer to a dose, dosage regimen, treatment or method disclosed herein. Efficacy can be measured based on change in the course of the disease in response to administering a composition disclosed herein. For example, a composition of the disclosure is administered to a subject in an amount and for a time sufficient to induce an improvement, preferably a sustained improvement, in at least one indicator that reflects the severity of cardiovascular disease. Various indicators that reflect the severity of the disease can be assessed for determining whether the amount and time of the treatment is sufficient. Such indicators include, for example, clinically recognized indicators of disease severity or symptoms. In an example, the degree of improvement is determined by a physician, who can make this determination based on signs, symptoms, or other test results (e.g. echocardiograph; LVEF; LVESV). In an example, a clinically proven effective amount improves patient survival. In another example, a clinically proven effective amount reduces a subjects risk of mortality. In another example, a clinically proven effective amount increases 100 day survival. In another example, a clinically proven effective amount increases LVEF. In an example, methods of the disclosure administer a clinically proven effective amount of a composition disclosed herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

### Mesenchymal Lineage Precursor or Stem Cells (MLPSCs)

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. As used herein, the terms “culture media” and “culture medium” are used interchangeably. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+ (TNAP+). For example, the MPCs can be isolated from bone mononuclear cells with an anti-STRO-3 antibody.

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as conditioned medium obtained therefrom under culture conditions. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy. Such extracellular vesicles can be characterised, if necessary, using one or more of the angiogenesis assays disclosed herein (e.g. endothelial network formation; endothelial length; endothelial branch length).

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

The term “pre-licensing” or “licensing”, as used herein, refers to a process by which MLPSCs achieve functional maturation, whereby, the pre-licensed or licensed MLPSCs reduce release of inflammatory cytokines when the MLPSCs are administered to a subject to a greater extent than MLPSCs that have not been pre-licensed.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for STRO-1+ cells comprises at least about 0.1% or 0.5% or 1% or 2% or 5% or 10% or 15% or 20% or 25% or 30% or 50% or 75% STRO-1+ cells. In this regard, the term “population of cells enriched for STRO-1+ cells” will be taken to provide explicit support for the term “population of cells comprising X % STRO-1+ cells”, wherein X % is a percentage as recited herein. The STRO-1+ cells can, in some examples, form clonogenic colonies, e.g. CFU-F (fibroblasts) or a subset thereof (e.g., 50% or 60% or 70% or 80% or 90% or 95%) can have this activity.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1^{bright}). Accordingly, an indication that cells are STRO-1+ does not mean that the cells are selected by STRO-1 expression. In one example, the cells are selected based on at least STRO-3 expression, e.g., they are STRO-3+ (TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the mesenchymal lineage precursor or stem cells used in the present disclosure express one or more markers individually or collectively selected from the group consisting of TNAP+, VCAM-1+, THY-1+, STRO-2+, STRO-4+ (HSP-90β), CD45+, CD146+, 3G5+ or any combination thereof.

By use of the term “individually” it is meant that the disclosure encompasses the recited markers or groups of markers separately, and that, notwithstanding that individual markers or groups of markers may not be separately listed herein the accompanying claims may define such marker or groups of markers separately and divisibly from each other.

By use of the term “collectively” it is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

In one example, the STRO-1+ cells are STRO-1^{bright} (syn. STRO-1^{bri}). In another example, the STRO-1^{bri} cells are preferentially enriched relative to STRO-1^{dim} or STRO-1^{intermediate} cells. In another example, the STRO-1^{bri} cells are additionally one or more of TNAP+, VCAM-1+, THY-1+, STRO-2+, STRO-4+ (HSP-90β) and/or CD146+. For example, the cells are selected for one or more of the foregoing markers and/or shown to express one or more of the foregoing markers. In this regard, a cell shown to express a marker need not be specifically tested, rather previously enriched or isolated cells can be tested and subsequently used, isolated or enriched cells can be reasonably assumed to also express the same marker.

In one example, the mesenchymal precursor cells are perivascular mesenchymal precursor cells as defined in WO 2004/85630, characterized by the presence of the perivascular marker 3G5.

A cell that is referred to as being “positive” for a given marker may express either a low (lo or dim) or a high (bright, bri) level of that marker depending on the degree to which the marker is present on the cell surface, where the terms relate to intensity of fluorescence or other marker used in the sorting process of the cells. The distinction of lo (or dim or dull) and bri will be understood in the context of the marker used on a particular cell population being sorted. A cell that is referred to as being “negative” for a given marker is not necessarily completely absent from that cell. This term means that the marker is expressed at a relatively very low level by that cell, and that it generates a very low signal when detectably labelled or is undetectable above background levels, e.g., levels detected using an isotype control antibody.

The term “bright” or “bri” as used herein, refers to a marker on a cell surface that generates a relatively high signal when detectably labelled. Whilst not wishing to be limited by theory, it is proposed that “bright” cells express more of the target marker protein (for example the antigen recognized by STRO-1) than other cells in the sample. For instance, STRO-1^{bri} cells produce a greater fluorescent signal, when labelled with a FITC-conjugated STRO-1 antibody as determined by fluorescence activated cell sorting (FACS) analysis, than non-bright cells (STRO-1^{dull/dim}). In one example, “bright” cells constitute at least about 0.1% of the most brightly labelled bone marrow mononuclear cells contained in the starting sample. In other examples, “bright” cells constitute at least about 0.5%, at least about 1%, at least about 1.5%, or at least about 2%, of the most brightly labelled bone marrow mononuclear cells contained in the starting sample. In an example, STRO-1^{bright} cells have 2 log magnitude higher expression of STRO-1 surface expression relative to “background”, namely cells that are STRO-1⁻. By comparison, STRO-1^{dim} and/or STRO-1^{intermediate} cells have less than 2 log magnitude higher expression of STRO-1 surface expression, typically about 1 log or less than “background”.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ multipotential cells are capable of differentiation into at least two different germ lines. Non-limiting examples of the lineages to which the multipotential cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an aspect of the present disclosure, the presently described mesenchymal lineage precursor or stem cells are MSCs. The MSCs may be a homogeneous composition or may be a mixed cell population enriched in MSCs. Homogeneous MSCs cell compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium.

In another example, the mesenchymal lineage precursor or stem cells are CD29+, CD54+, CD73+, CD90+, CD102+, CD105+, CD106+, CD166+, MHC1+ MSCs (e.g. remestemcel-L).

As will be appreciated by those of skill in the art, cultured mesenchymal lineage precursor or stem cells are phenotypically different to cells in-vivo. For example, in one embodiment they express one or more of the following markers, CD44, NG2, DC146 and CD140b. Cultured mesenchymal lineage precursor or stem cells are also biologically different to cells in-vivo, having a higher rate of proliferation compared to the largely non-cycling (quiescent) cells in-vivo.

Mesenchymal lineage precursor or stem cells cultured using the methods of the present disclosure may also be cryopreserved.

Culture Expanded MLPSC and Conditioned Media Obtained from the Same

In example, culture expanded MLPSCs of the disclosure and/or conditioned media obtained from the same are characterised by expression of an angiogenic marker(s). For example, a culture expanded MLPSC population according to the present disclosure and/or conditioned media obtained from the same can be characterised by increased levels of VEGF, angiogenin, and/or SDF-1a under culture conditions. In another example, the MLPSC population can be characterised based on an assessment of conditioned media obtained from the MLPSC population under culture conditions. In an example, the conditioned media increases the level endothelial network formation, endothelial network length, and/or endothelial branch length in a population of endothelial cells when said cells are treated with conditioned media obtained from culture expanded MLPSCs. In an example, the increase is determined relative to a control population of MLPSCs. In an example, the control population is a population of MLPSCs that have been culture expanded in a cell culture medium comprising 10% fetal calf serum.

In an example, the expanded MLPSC population is characterised by a level of VEGF greater than about 3 ng/ml. In an example, the level of VEGF is between about 3 ng/ml to 4 ng/ml. In an example, the level of VEGF is greater than about 3.1 ng/ml. In an example, level of VEGF is greater than about 3.2 ng/ml. In an example, level of VEGF is greater than about 3.3 ng/ml. In an example, the level of VEGF is greater than about 3.4 ng/ml. In an example, the level of VEGF is greater than about 3.5 ng/ml. In an example, the level of VEGF is between about 3.2 and 3.6 ng/ml. In an example, the level of VEGF is about 3.45 ng/mL.

In an example, the MLPSCs express an increased level of angiogenin relative to a control population. In an example, the expanded MLPSC population is characterised by a level of angiogenin greater than about 1000 pg/ml. In an example, the level of angiogenin is greater than about 1100 pg/ml. In an example, the level of angiogenin is between about 1000 pg/ml and 1200 pg/ml. In an example, the level of angiogenin is between about 1100 pg/ml and 1150 pg/ml. In an example, the level of angiogenin is about 1114 pg/ml.

In an example, the expanded MLPSC population is characterised by a level of SDF-1a greater than about 3000 ng/ml. In an example, the level of SDF-1a is greater than about 3100 ng/ml. In an example, the level of SDF-1a is greater than about 3200 ng/ml. In an example, the level of SDF-1a is greater than about 3300 ng/ml. In an example, the level of SDF-1a is greater than about 3400 ng/ml. In an example, the level of SDF-1a is greater than about 3500 ng/ml. In an example, the level of SDF-1a is between about 3000 ng/ml and 3500 ng/ml. In an example, the level of SDF-1a is between about 3000 ng/ml and 3400 ng/ml. In an example, the level of SDF-1a is between about 3000 ng/ml and 3300 ng/ml. In an example, the level of SDF-1a is between about 3100 ng/ml and 3400 ng/ml. In an example, the level of SDF-1a is between about 3100 ng/ml and 3300 ng/ml.

In an example, the culture expanded MLPSC population is characterised by conditioned media which stimulates endothelial network formation greater than about 0.1 mm²/mm². In an example, the endothelial network formation is between about 0.1 mm²/mm² and 0.2 mm²/mm². In another example, the endothelial network formation is about 0.12 mm²/mm².

In an example, the culture expanded MLPSC population is characterised by conditioned media which stimulates endothelial network length greater than about 4 mm²/mm². In an example, the endothelial network length is between about 4 mm²/mm² and about 6 mm²/mm². In an example, the endothelial network length is about 5 mm²/mm². In an example, the culture expanded MLPSC population is characterised by conditioned media which stimulates endothelial branch length greater than about 12 l/mm². In an example, the endothelial branch length is between about 12 l/mm² and about 17 l/mm². In an example, the endothelial branch length is about 15 l/mm².

In an example, the culture expanded MLPSC population is characterised by an increased level of one or more angiogenic markers relative to a population of MLPSCs that have been culture expanded in a cell culture medium comprising 10% fetal serum (e.g. fetal calf serum). In an example, the level of angiogenic marker is increased by about 5%, about 10%, about 20%, about 30%, about 40%, about 50%, about 60%, or about 70% relative to a population of MLPSCs that have been culture expanded in a cell culture medium comprising 10% fetal serum (e.g. fetal calf serum). In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%. In an example, the level of angiogenic marker is increased relative to a population of MLPSCs that have been culture expanded in cell culture medium that does not contain IFN-gamma or TNF-alpha.

In an example, the expanded MLPSC population is characterised by an increased level of one or more angiogenic markers relative to a population of MLPSCs that have been culture expanded in a cell culture medium that does not comprise newborn serum. In an example, the level of angiogenic marker is increased by about 5%, about 10%, about 20%, about 30%, about 40%, about 50%, about 60%, or about 70% relative to a population of MLPSCs that have been culture expanded in a cell culture medium that does not comprise newborn serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In another example, culture expanded MLPSCs of the disclosure are characterised based on therapeutic efficacy. For example, the MLPSCs may be characterised based on therapeutic efficacy in an inflammatory disease. In an example, the MLPSCs are characterised by therapeutic efficacy in heart failure. In another example, the MLPSCs are characterised by therapeutic efficacy in a T-cell mediated disease such as GvHD.

In another example, culture expanded MLPSCs are characterised by their capacity to inhibit IL-2RA expression by CD3/CD28-activated PBMCs under culture conditions. In an example, the culture expanded MLPSCs inhibit IL2-RA expression by CD3/CD28-activated PBMCs by at least 60% relative to a control. In another example, the culture expanded MLPSCs inhibit IL2-RA expression by CD3/CD28-activated PBMCs by at least 65% relative to a control. In another example, the culture expanded MLPSCs inhibit IL2-RA expression by CD3/CD28-activated PBMCs by at least 70% relative to a control. In another example the culture expanded MLPSCs inhibit IL2-RA expression by CD3/CD28-activated PBMCs by between 60 and 70% relative to a control.

“Culture expanded” MLPSCs are distinguished from freshly isolated cells in that they have been cultured in cell culture medium and passaged (i.e. sub-cultured).

In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population. In an example, freshly isolated cells are STRO-1+.

Accordingly, in an example, relevant cells are isolated and culture expanded for 2 passages to provide an intermediate MLPSC population. In certain examples, the intermediate MLPSC population is then culture expanded to provide a drug product (DP). For example, DP compositions of the present disclosure are produced by culturing cells from an intermediate cryopreserved MLPSC population or, put another way, a cryopreserved intermediate. In an example, the intermediate cell population can be cultured for three more passages (i.e. 5 passages total) to provide a DP.

In an example, MLPSCs are culture expanded for about 4-10 passages. In an example, MLPSCs are culture expanded for at least 5, at least 6, at least 7, at least 8, at least 9, at least 10 passages. For example, MLPSCs can be culture expanded for at least 5 passages. In an example, MLPSCs can be culture expanded for at least 5-10 passages. In an example, MLPSCs can be culture expanded for at least 5-8 passages. In an example, MLPSCs can be culture expanded for at least 5-7 passages. In an example, MLPSCs can be culture expanded for more than 7 passages. In these examples, MLPSCs may be culture expanded before being cryopreserved to provide an intermediate cryopreserved MLPSC population and then subject to further culture expansion.

In an example, compositions of the disclosure comprise MLPSCs that are culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, MLPSCs can be culture expanded for at least 3 passages. In an example, MLPSCs can be culture expanded for at least 3-10 passages. In an example, MLPSCs can be culture expanded for at least 3-8 passages. In an example, MLPSCs can be culture expanded for at least 3-7 passages. In an example, MLPSCs culture expanded from a cryopreserved intermediate are culture expanded in media disclosed herein (e.g. media containing newborn calf serum).

In an example, MLPSCs can be obtained from a single donor, or multiple donors where the donor samples or MLPSCs are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated MLPSCs and then serially establishing a first non-primary (P1) culture of isolated MLPSCs from the previous culture;
ii. expanding by passage expansion the P1 culture of isolated MLPSCs to a second non-primary (P2) culture of MLPSCs; and,
iii. preparing and cryopreserving an in-process intermediate MLPSC preparation obtained from the P2 culture of MLPSCs; and, optionally
iv. thawing the cryopreserved in-process intermediate MLPSC preparation and expanding by passage expansion the in-process intermediate MLPSC preparation.

In an example, the methods of the disclosure comprise selecting an intermediate population (e.g. a cryopreserved intermediate) for further culture expansion based on certain criteria such as the level of one more angiogenic markers. Selection processes are not particularly limited so long as they are able to select cell populations characterized by the relevant criteria such as level of angiogenic marker. In an example, a series of intermediate MLPSC populations are assessed for levels of angiogenic markers and those populations which express over a threshold level of the angiogenic marker as described herein are selected for further expansion.

It should be appreciated that the selection process does not require immediate culture expansion. Rather “selected” populations can be cryopreserved and culture expanded at a later stage. In an example, a fraction of the intermediate cell population is culture expanded with the remainder of the population being cryopreserved for culture expansion at a later stage.

In an example, selected cell populations are immediately culture expanded. In another example, selected cell populations are cryopreserved to allow culture expansion at a later stage.

In an example, a selected cell population is culture expanded to provide a pharmaceutical composition. In an example, the pharmaceutical composition is characterized by certain criteria such as level of angiogenic markers.

In the context of the present disclosure, the level of angiogenic marker/s can be assessed between steps iii and iv of the culture expansion process described above. For example, the level of angiogenic marker/s may be determined under culture conditions and/or from conditioned media after step iii. In an example, step iv is only performed if a desired level of angiogenic marker/s is/are observed under culture conditions and/or from conditioned media. In this example, the cell population is selected for culture expansion on the basis of the level of angiogenic marker/s under culture conditions and/or from conditioned media.

In an example, the culture expanded MLPSC population is expanded from an intermediate MLPSC population with an increased level of one or more angiogenic markers relative to a population of MLPSCs that have been culture expanded in a cell culture medium comprising 10% fetal calf serum.

In an example, a level of an angiogenic marker(s) disclosed herein is considered increased when it is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, the culture expanded MLPSC population is expanded from an intermediate MLPSC population with an increased level of one or more angiogenic markers relative to a population of MLPSCs that have been culture expanded in a cell culture medium that does not comprise newborn serum. In an example, the level of angiogenic marker is considered increased when it is increased by about 5%, about 10%, about 20%, about 30%, about 40%, about 50%, about 60%, or about 70% relative to a population of MLPSCs that have been culture expanded in a cell culture medium that does not comprise newborn serum. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, the culture expanded MLPSC preparation has an antigen profile and an activity profile comprising:
i. less than about 0.75% CD45+ cells;
ii. at least about 95% CD105+ cells;
iii. at least about 95% CD166+ cells.

### Conditioned Media

In an example, conditioned media or extracellular vesicles obtained therefrom are characterised by expression of an angiogenic marker(s). For example, conditioned media or extracellular vesicles obtained therefrom can be characterised by increased levels of VEGF, angiogenin, and/or SDF-1a under culture conditions. In another example, conditioned media or extracellular vesicles obtained therefrom can be characterised based on one or more functional criteria. In an example, the conditioned media or extracellular vesicles obtained therefrom increases the level endothelial network formation, endothelial network length, and/or endothelial branch length in a population of endothelial cells when said cells are treated with conditioned media or extracellular vesicles obtained therefrom that have been obtained from culture expanded MLPSCs. In an example, the increase is determined relative to conditioned media or extracellular vesicles obtained therefrom which have been obtained from a control population of MLPSCs. In an example, the control population is a population of MLPSCs that have been culture expanded in a cell culture medium comprising 10% fetal calf serum.

In an example, the conditioned media is characterised by a level of VEGF greater than about 3 ng/ml. In an example, the level of VEGF is between about 3 ng/ml to 4 ng/ml. In an example, the level of VEGF is greater than about 3.1 ng/ml. In an example, level of VEGF is greater than about 3.2 ng/ml. In an example, level of VEGF is greater than about 3.3 ng/ml. In an example, the level of VEGF is greater than about 3.4 ng/ml. In an example, the level of VEGF is greater than about 3.5 ng/ml. In an example, the level of VEGF is between about 3.2 and 3.6 ng/ml. In an example, the level of VEGF is about 3.45 ng/mL.

In an example, the conditioned media or extracellular vesicles obtained therefrom contain an increased level of angiogenin relative to a control population. In an example, the conditioned media is characterised by a level of angiogenin greater than about 1000 pg/ml. In an example, the level of angiogenin is greater than about 1100 pg/ml. In an example, the level of angiogenin is between about 1000 pg/ml and 1200 pg/ml. In an example, the level of angiogenin is between about 1100 pg/ml and 1150 pg/ml. In an example, the level of angiogenin is about 1114 pg/ml.

In an example, the conditioned media is characterised by a level of SDF-1a greater than about 3000 ng/ml. In an example, the level of SDF-1a is greater than about 3100 ng/ml. In an example, the level of SDF-1a is greater than about 3200 ng/ml. In an example, the level of SDF-1a is greater than about 3300 ng/ml. In an example, the level of SDF-1a is greater than about 3400 ng/ml. In an example, the level of SDF-1a is greater than about 3500 ng/ml. In an example, the level of SDF-1a is between about 3000 ng/ml and 3500 ng/ml. In an example, the level of SDF-1a is between about 3000 ng/ml and 3400 ng/ml. In an example, the level of SDF-1a is between about 3000 ng/ml and 3300 ng/ml. In an example, the level of SDF-1a is between about 3100 ng/ml and 3400 ng/ml. In an example, the level of SDF-1a is between about 3100 ng/ml and 3300 ng/ml.

In an example, the conditioned media stimulates endothelial network formation greater than about 0.1 mm²/mm². In an example, the endothelial network formation is between about 0.1 mm²/mm² and 0.2 mm²/mm². In another example, the endothelial network formation is about 0.12 mm²/mm².

In an example, the conditioned media stimulates endothelial network length greater than about 4 mm²/mm². In an example, the endothelial network length is between about 4 mm²/mm² and about 6 mm²/mm². In an example, the endothelial network length is about 5 mm²/mm². In an example, the conditioned media stimulates endothelial branch length greater than about 12 l/mm². In an example, the endothelial branch length is between about 12 l/mm² and about 17 l/mm². In an example, the endothelial branch length is about 15 l/mm².

In an example, the conditioned media or extracellular vesicles obtained therefrom is characterised by an increased level of one or more angiogenic markers relative to conditioned media or extracellular vesicles obtained therefrom that have been obtained from a population of MLPSCs that have been culture expanded in a cell culture medium comprising 10% fetal calf serum. In an example, the level of angiogenic marker is increased by about 5%, about 10%, about 20%, about 30%, about 40%, about 50%, about 60%, or about 70%. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%. In an example, the level of angiogenic marker is increased relative to conditioned media or extracellular vesicles obtained therefrom that have been obtained from a population of MLPSCs that have been culture expanded in cell culture medium that does not contain IFN-gamma or TNF-alpha.

In an example, the conditioned media or extracellular vesicles obtained therefrom is characterised by an increased level of one or more angiogenic markers relative to conditioned media or extracellular vesicles obtained therefrom that have been obtained from a population of MLPSCs that have been culture expanded in a cell culture medium that does not comprise newborn serum. In an example, the level of angiogenic marker is increased by about 5%, about 10%, about 20%, about 30%, about 40%, about 50%, about 60%, or about 70%. In an example, the level of angiogenic marker is increased by between about 5% and about 60%. In an example, the level of angiogenic marker is increased by between about 5% and about 40%. In an example, the level of angiogenic marker is increased by about 40%. In an example, the level of angiogenic marker is increased by at least about 5%. In an example, the level of angiogenic marker is increased by at least about 10%.

In an example, freshly isolated cells are culture expanded for about 1 or 2 passages to provide an intermediate population. In an example, freshly isolated cells are culture expanded for 2 passages to provide an intermediate population. In another example, freshly isolated cells are culture expanded for about 1 to 3 passages to provide an intermediate population. In an example, freshly isolated cells are STRO-1+. In an example, conditioned media or extracellular vesicles obtained therefrom are produced by culture expanding cells to provide a cryopreserved intermediate.

Accordingly, in an example, relevant cells are isolated and culture expanded for 2 passages to provide an intermediate MLPSC population. In certain examples, the intermediate MLPSC population is then culture expanded to provide a drug product (DP). In an example, conditioned media or extracellular vesicles obtained therefrom are obtained from DP MLPSCs.

In an example, MLPSCs are culture expanded for about 4-10 passages to provide conditioned media or extracellular vesicles obtained therefrom. In an example, MLPSCs are culture expanded for at least 5, at least 6, at least 7, at least 8, at least 9, at least 10 passages to provide conditioned media or extracellular vesicles obtained therefrom. For example, MLPSCs can be culture expanded for at least 5 passages to provide conditioned media or extracellular vesicles obtained therefrom. In an example, MLPSCs can be culture expanded for at least 5-10 passages to provide conditioned media or extracellular vesicles obtained therefrom. In an example, MLPSCs can be culture expanded for at least 5-8 passages to provide conditioned media or extracellular vesicles obtained therefrom. In an example, MLPSCs can be culture expanded for at least 5-7 passages to provide conditioned media or extracellular vesicles obtained therefrom. In an example, MLPSCs can be culture expanded for more than 7 passages to provide conditioned media or extracellular vesicles obtained therefrom. In these examples, MLPSCs may be culture expanded before being cryopreserved to provide an intermediate cryopreserved MLPSC population and then subject to further culture expansion to provide conditioned media or extracellular vesicles obtained therefrom.

In an example, conditioned media or extracellular vesicles obtained therefrom are obtained from MLPSCs that have been culture expanded from a cryopreserved intermediate. In an example, the cells culture expanded from a cryopreserved intermediate are culture expanded for at least 3, at least 5, at least 6, at least 7, at least 8, at least 9, at least 10 passages. For example, MLPSCs can be culture expanded for at least 3 passages. In an example, MLPSCs can be culture expanded for at least 3-10 passages. In an example, MLPSCs can be culture expanded for at least 3-8 passages. In an example, MLPSCs can be culture expanded for at least 3-7 passages. In an example, MLPSCs culture expanded from a cryopreserved intermediate are culture expanded in media disclosed herein (e.g. media containing newborn calf serum).

In an example, MLPSCs can be obtained from a single donor, or multiple donors where the donor samples or MLPSCs are subsequently pooled and then culture expanded as required. In an example, the culture expansion process comprises:
i. expanding by passage expansion the number of viable cells to provide a preparation of at least about 1 billion of the viable cells, wherein the passage expansion comprises establishing a primary culture of isolated MLPSCs and then serially establishing a first non-primary (P1) culture of isolated MLPSCs from the previous culture;
ii. expanding by passage expansion the P1 culture of isolated MLPSCs to a second non-primary (P2) culture of MLPSCs; and,
iii. preparing and cryopreserving an in-process intermediate MLPSC preparation obtained from the P2 culture of MLPSCs; and, optionally
iv. thawing the cryopreserved in-process intermediate MLPSC preparation and expanding by passage expansion the in-process intermediate MLPSC preparation.

In an example, the methods of the disclosure comprise selecting an intermediate population (e.g. a cryopreserved intermediate) for further culture expansion based on certain criteria such as the level of one more angiogenic markers. Selection processes are not particularly limited so long as they are able to select cell populations characterized by the relevant criteria such as level of angiogenic marker. In an example, a series of intermediate MLPSC populations are assessed for levels of angiogenic markers and those populations which express over a threshold level of the angiogenic marker as described herein are selected for further expansion.

It should be appreciated that the selection process does not require immediate culture expansion. Rather “selected” populations can be cryopreserved and culture expanded at a later stage. In an example, a fraction of the intermediate cell population is culture expanded with the remainder of the population being cryopreserved for culture expansion at a later stage.

In an example, selected cell populations are immediately culture expanded. In another example, selected cell populations are cryopreserved to allow culture expansion at a later stage.

In an example, conditioned media or extracellular vesicles obtained therefrom are obtained from a culture expanded MLPSC population that has an antigen profile and an activity profile comprising:
i. less than about 0.75% CD45+ cells;
ii. at least about 95% CD105+ cells;
iii. at least about 95% CD166+ cells.

### General MLPSC Expansion Methods

The process of MLPSC isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although MLPSCs can generally withstand a certain amount of insult during preparation, cells are preferably manipulated by handling procedures and/or equipment that adequately performs the given step(s) while minimizing insult to the cells.

In an example, MLPSCs are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a MLPSC composition cultured according to the present disclosure is 95% homogeneous with respect to being CD105 positive and CD166 positive and being CD45 negative. In an example, this homogeneity persists through ex vivo expansion; i.e. though multiple population doublings.

In an example, MLPSCs of the disclosure are culture expanded in 2D culture. For example, MLPSCs of the disclosure can be culture expanded in a cell factory. In certain examples, 3D culture of intermediates disclosed herein may follow using, for example, a bioreactor. In an example, MLPSCs of the disclosure are initially culture expanded in 2D culture prior to being further expanded in 3D culture. In an example, intermediate cell populations of the disclosure have not been culture expanded in 3D culture. In an example, the level of one or more angiogenic markers is assessed before subsequent culture expansion in a cell factory or 3D culture.

In an example, MLPSCs of the disclosure are culture expanded from an intermediate population. In an example, MLPSCs of the disclosure are culture expanded from the intermediate in 2D culture before seeding in 3D culture.

In the context of both intermediate populations and therapeutic compositions expanded from the same, in an example, MLPSCs of the disclosure are culture expanded in 2D culture for at least 3 days before seeding in a further culture system such as cell factory or 3D culture in a bioreactor. In an example, MLPSCs of the disclosure are culture expanded in 2D culture for at least 4 days before seeding in a further culture system. In an example, MLPSCs of the disclosure are culture expanded in 2D culture for between 3 and 5 days before seeding in a further culture system. In these examples, 2D culture can be performed in a cell factory. Various cell factory products are available commercially (e.g. Thermofisher, Sigma, Corning). In an example, the cell factory has at least 5 layers. In an example, the cell factory has at least 10 layers. In an example, the cell factory has at least 20 layers. 3D culture may be performed in various bioreactor types such as stirred tank, wave bag, and vertical wheel.

In an example, CO₂ is provided during culture expansion of MLPSCs. In an example, MLPSCs are culture expanded in less than 9% CO₂. In an example, MLPSCs are culture expanded in less than 8% CO₂. In an example, MLPSCs are culture expanded in 5% CO₂. For example, MLPSCs can be culture expanded in 5%+/−2% CO₂. In an example, the MLPSCs are culture expanded with passive priming of CO₂. For example, cell factories can be passively primed with 5% CO₂.

Priming cell factories maintains the CO₂ tension between the cell factory and incubator and stabilizes the pH level of the growth medium. Active priming involves actively passing CO₂ gas through a bacterial vent air filter into each culture vessel (e.g. cell factory) for a defined period of time (e.g. around 10 minutes). However, active priming has the potential to introduce contamination into culture as it requires an open port to provide gas. Passive priming involves placing a closed culture system into an incubator at appropriate CO₂ concentration prior to cell seeding (e.g. around 12 to 72 hours). In an example, cells of the disclosure are STRO-3+ before they are culture expanded to provide an intermediate cell population.

### Cell Culture Methods

Compositions of the disclosure can be prepared via culture expansion in media containing one or more pro-inflammatory cytokines and/or a non-fetal serum disclosed herein, such as newborn serum.

For example, MLPSC culture media can be supplemented with pro-inflammatory cytokine(s). In an example, the culture media comprises IFN-gamma and/or TNF-alpha. In an example, the media comprises IFN-gamma. For example, the level of IFN-gamma can be less than 1 ng/ml. In an example the level of IFN-gamma is less than 500 pg/ml or less than 100 pg/ml. In an example, the media comprises TNF-alpha. For example, the level of TNF-alpha can be less than 1 ng/ml. In an example, the level of TNF-alpha is less than 750 pg/ml or less than 400 pg/ml. In an example, the media comprises IFN-gamma and TNF-alpha and the level of both is less than 1 ng/ml.

In an example, the media comprises one or more pro-inflammatory cytokines which are capable of binding a receptor on the surface of MLPSCs.

In an example, the media comprises one or more pro-inflammatory cytokines selected from the group consisting of IL-6; IL-8; IL-17A; MCP-1; MIP-1-alpha; MIP-1-beta; IP-10. For example, the media can comprise IL-8.

In an example, the media comprises IFN-gamma and/or TNF-alpha, and, one or more pro-inflammatory cytokines selected from the group consisting of IL-6; IL-8; IL-17A; MCP-1; MIP-1-alpha; MIP-1-beta; IP-10. In an example, the level of IFN-gamma and/or TNF-alpha is less than 1 ng/ml.

In an example, the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

In another example, the media comprises serum which is characterised by one or more or all of the following:
i. a level of IFN-gamma greater than 10 pg/ml;
ii. a level of TNF-alpha greater than 20 pg/ml;
iii. a level of IL-6 greater than 30 pg/ml;
iv. a level of IL-8 greater than 5,000 pg/ml;
V. a level of IL-17A greater than 2 pg/ml;
vi. a level of MCP-1 greater than 30 pg/ml;
vii. a level of MIP-1-alpha greater than 50 pg/ml;
viii. a level of MIP-1-beta greater than 30 pg/ml;
ix. a level of IP-10 greater than 5,000 pg/ml.

In an example, the media comprises IL-10. In another example, the media comprises IL-36RA. In another example, the media comprises IL-10 and IL-36RA. In an example, the level of IL-10 is greater than 0.3 pg/ml. For example, the level of IL-10 may be greater than 30 pg/ml. In an example, the level of IL-10 is greater than 400 pg/ml. In an example, the level of IL-36RA is greater than 50 pg/ml.

In an example, the media is serum free.

In an example, the media is serum free and supplemented with PDGF and FGF2. In an example, the medium is serum free and is supplemented with PDGF, FGF2 and EGF. In an example, the PDGF is PDGF-BB. In an example, the serum free media is supplemented with 10 ng/ml PDGF-BB, 5 ng/ml EGF and, 1 ng/ml FGF2.

In an example, the above referenced cytokines can be provided at a concentration <1 ng/ml each. For example, the media may be characterised by one or more or all of the following, each provided at <1 ng/ml: IFN-gamma, TNF-alpha, IL-6, IL-17A, MCP-1, MIP-1-alpha, MIP-1-beta, IP-10.

In another example, compositions of the disclosure can be prepared via culture expansion in a culture medium that is supplemented with a serum comprising one or more pro-inflammatory cytokines as described herein. In some preferred embodiments, the culture medium is supplemented with a non-fetal serum, such as newborn serum. In some preferred embodiments, the culture medium is supplemented with both fetal serum and newborn serum in equal concentrations for a total serum concentration in the culture medium of about 10% (v/v). In some preferred embodiments MLPSCs are pre-licensed in cell culture medium containing 5% (v/v) newborn serum and 5% (v/v) fetal serum.

In some embodiments the methods of preparing MLPSCs disclosed herein include the additional step of determining or having determined the level of one or more pro-inflammatory cytokines in a serum to be included in the culture medium to be used for pre-licensing of MLPSCs. Methods for determining cytokine levels are well known in the art, e.g., ELISA.

In some embodiments the methods of preparing MLPSCs disclosed herein also include determining or having determined the ability of a culture medium (e.g. a newborn serum supplemented culture medium) to stimulate MLPSCs to promote angiogenesis in an in vitro assay, e.g., tube formation by human umbilical vein endothelial cells (HUVEC) and analysis of network length, network area and branch point formation. In some embodiments such an assay includes collecting MLPSC-conditioned media following its culture in a newborn serum-supplemented medium as disclosed herein and quantifying the effect of such conditioned media in the above-described angiogenesis assay or a similar assay.

In some embodiments the methods of preparing MLPSCs disclosed herein also include determining or having determined in the above-mentioned conditioned medium the level of one or more of Angiogenin, Angiopoietin (Ang1/ANGPT1), SDF-1α, and VEGF.

In some embodiments, where a first lot or batch of newborn serum was used in conditioned medium that promotes greater angiogenesis or release of angiogenic factors than that of conditioned medium in which second lot/batch of newborn serum was used, it is concluded that the use of the first lot of newborn serum for pre-licensing and culture expansion of MLPSCs will result in the generation of MLPSCs that have relatively greater therapeutic potency particularly for treatment of conditions where an angiogeneic or anti-inflammatory therapeutic mode of action is useful.

The methods and cell culture media used to prepare MLPSCs of the disclosure promote stem cell proliferation while maintaining MLPSCs in an undifferentiated state. MLPSCs are considered to be undifferentiated when they have not committed to a specific differentiation lineage. As discussed above, MLPSCs display morphological characteristics that distinguish them from differentiated cells. Furthermore, undifferentiated MLPSCs express genes that may be used as markers to detect differentiation status. The polypeptide products may also be used as markers to detect differentiation status. Accordingly, one of skill in the art could readily determine whether the methods of the present disclosure maintain MLPSCs in an undifferentiated state using routine morphological, genetic and/or proteomic analysis. Methods of monitoring/confirming cell proliferation are also known in the art and, in certain examples, may be as rudimentary as periodic visual inspection of cell cultures to confirm increase in cell number. Other methods may involve the use of cell viability dyes and/or live cell imaging and counting using commercially available products.

MLPSCs disclosed herein can be culture expanded in various suitable cell culture mediums comprising newborn serum. The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In the above examples, basal medium such as Alpha MEM or StemSpan™ can be supplemented with the referenced quantity of serum and, in certain examples, other additives. Further examples of suitable culture mediums for culturing stem cells can be found, for example, in WO2016139340.

### Serum

“Non-fetal serum” refers to serum that has been obtained postpartum. For example, the culture media can be supplemented with mammalian non-fetal serum (e.g. bovine). In an example, the culture media can be supplemented with an animal non-fetal serum. In another example, the culture media can be supplemented with human non-fetal serum.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v non-fetal serum. In an example, the cell culture media is supplemented with between about 1% v/v and about 15% v/v non-fetal serum. In an example, the cell culture media is supplemented with between about 1% v/v and about 10% v/v non-fetal serum. In an example, the cell culture media is supplemented with between about 5% v/v and about 10% v/v non-fetal serum. In an example, the cell culture media is supplemented with between about 5% v/v non-fetal serum.

In an example, the non-fetal serum comprises at least one pro-inflammatory cytokine. Methods to detect the presence of cytokines in cell culture medium and/or serum are known in the art and include, for example, enzyme-linked immunosorbent assay (ELISA). In another example, the presence of cytokines in serum are detected by measuring cytokine mRNA, for example by polymerase-chain reaction (PCR) techniques such as reverse-transcription PCR.

In an example, the non-fetal serum is a newborn serum such as newborn calf serum. “Newborn serum” refers to serum that has been obtained postpartum. For example, the culture media can be supplemented with mammalian newborn serum (e.g. bovine). In an example, the culture media can be supplemented with animal newborn serum.

In an example, the newborn serum is obtained within 4 weeks after birth of the animal. In an example, the newborn serum is obtained within 21 days after birth of the animal. For example, the newborn serum is obtained ≤21 days after birth of the animal. In an example, the newborn serum is obtained between the day of birth and 21 days after birth of the animal. In an example, the newborn serum is obtained between the day of birth and 14 days after birth of the animal. In an example, the newborn serum is obtained between the day of birth and 10 days after birth of the animal. In an example, the newborn serum is obtained between the day of birth and 7 days after birth of the animal. In an example, the newborn serum is obtained between 6 hours after birth and 72 hours after birth. In an example, the newborn serum is obtained between 6 hours after birth and 48 hours after birth. In an example, the newborn serum is obtained between 6 hours after birth and 24 hours after birth. In an example, the newborn serum is obtained between 12 hours after birth and 24 hours after birth.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v newborn serum. In an example, the cell culture media is supplemented with between about 1% v/v and about 15% v/v newborn serum. In an example, the cell culture media is supplemented with between about 1% v/v and about 10% v/v newborn serum. In an example, the cell culture media is supplemented with between about 5% v/v and about 10% v/v newborn serum. In an example, the cell culture media is supplemented with about 5% v/v newborn serum.

In an example, the newborn serum comprises at least one inflammatory cytokine. As used herein, the term “inflammatory cytokine” refers to a signalling molecule that promotes inflammation. In example, the one or more cytokine is selected from the group comprising IL-1B, IL-6, TNF-α, IFN-γ and/or IL-1ra.

In an example, the newborn serum comprises IFN-gamma. In another example, the newborn serum comprises TNF-alpha. In another example, the newborn serum comprises IFN-gamma and TNF-alpha. In another example, the newborn serum comprises one or more pro-inflammatory cytokines selected from the group consisting of IL-6; IL-8; IL-17A; MCP-1; MIP-1-alpha; MIP-1-beta; IP-10. For example, the newborn serum can comprise IL-8. In an example, the newborn serum comprises IFN-gamma and/or TNF-alpha and, one or more pro-inflammatory cytokines selected from the group consisting of IL-6; IL-8; IL-17A; MCP-1; MIP-1-alpha; MIP-1-beta; IP-10. In another example, the newborn serum comprises IFN-gamma and TNF-alpha and, one or more pro-inflammatory cytokines selected from the group consisting of IL-6; IL-8; IL-17A; MCP-1; MIP-1-alpha; MIP-1-beta; IP-10. In an example, the level of IFN-gamma is less than 1 ng/ml. In an example, the level of TNF-alpha is less than 1 ng/ml. In an example, the level of both IFN-gamma and TNF-alpha are less than 1 ng/ml. For example, the level of IFN-gamma may be less than 500 pg/ml or less than 100 pg/ml. In an example, the level of TNF-alpha is less than 750 pg/ml or less than 400 pg/ml.

Methods to detect the presence of cytokines in serum are known in the art and include, for example, enzyme-linked immunosorbent assay (ELISA). In another example, the presence of cytokines in serum are detected by measuring cytokine mRNA, for example by polymerase-chain reaction (PCR) techniques such as reverse-transcription PCR.

In an example, the newborn serum can be newborn calf serum (NBCS). In an example, the NBCS is obtained from newborn calves who have been fed colostrum. In an example, NBCS comprises elevated levels of at least one inflammatory cytokine relative to NBCS obtained from a calf that has not been fed colostrum. In an example, NBCS comprises elevated levels of at least one inflammatory cytokine relative to fetal serum such as FCS.

In an example, the NBCS is obtained within 4 weeks after birth of the calf. In an example, the NBCS is obtained within 21 days after birth of the calf. For example, the NBCS is obtained ≤21 days after birth of the calf. In an example, the NBCS is obtained between the day of birth and 21 days after birth of the calf. In an example, the NBCS is obtained between the day of birth and 14 days after birth of the calf. In an example, the NBCS is obtained between the day of birth and 10 days after birth of the calf. In an example, the NBCS is obtained between the day of birth and 7 days after birth of the calf. In an example, the NBCS is obtained between 6 hours after birth and 72 hours after birth. In an example, the NBCS is obtained between 6 hours after birth and 48 hours after birth. In an example, the NBCS is obtained between 6 hours after birth and 24 hours after birth. In an example, the NBCS is obtained between 12 hours after birth and 24 hours after birth.

In an example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v NBCS. In an example, the cell culture media is supplemented with between about 1% v/v and about 15% v/v NBCS. In an example, the cell culture media is supplemented with between about 5% v/v and about 10% v/v NBCS. In an example, the cell culture media is supplemented with at least about 5% v/v NBCS.

In an example, the culture medium is also supplemented with fetal serum. In an example, the fetal serum is fetal calf serum (FCS). It is envisaged that the term fetal calf serum (FCS) and fetal bovine serum (FBS) can in the context of the present disclosure be used interchangeably. In an example, cell culture medium is supplemented with less than 10% v/v FCS. In an example, cell culture medium is supplemented with about 5% v/v FCS.

In an example, the cell culture medium is fetal serum free.

In an example, the cell culture medium is FCS free.

In an example, the culture media is supplemented with a mixture of FCS and NBCS. In an example the cell culture medium is supplemented with about 5% v/v FCS and about 5% v/v NBCS (i.e. a 1:1 ratio of FCS to NBCS). In an example, the culture media can be supplemented with a mixture of FCS and NBCS so that the FCS: NBCS ratio is at least about 0.4:1, at least about 0.5:1, at least about 0.6:1, at least about 0.7:1, at least about 0.8:1, at least about 0.9:1, at least about 1:1, at least about 1.5:1, at least about 2:1. In an example, the FCS: NBCS ratio is between about 0.5:1 and about 2:1. In an example, the FCS: NBCS ratio is between about 0.8:1 and about 1.5:1. In an example, the FCS: NBCS ratio is between about 0.8:1 and about 1.2:1. In an example, the FCS: NBCS ratio is about 1:1.

In an example, the mixture of FCS and NBCS can comprise at least about 1% v/v, at least about 2% v/v, at least about 3% v/v, at least about 4% v/v, at least about 5% v/v, at least about 6% v/v, at least about 7% v/v, at least about 8% v/v, at least about 9%, at least about 10%, at least about 11%, at least about 12%, at least about 13%, at least about 14%, at least about 15%, at least about 16%, at least about 17%, at least about 18%, at least about 19%, at least about 20%, at least about 21%, at least about 22%, at least about 23%, at least about 24%, at least about 25% v/v of the cell culture media. In an example, the mixture of FCS and NBCS can comprise between about 1% v/v and about 15% v/v of the cell culture media. In an example, the mixture of FCS and NBCS can comprise between about 2% v/v and about 12% v/v of the cell culture media. In an example, the mixture of FCS and NBCS can comprise between about 5% v/v and about 12% v/v of the cell culture media. In an example, the mixture of FCS and NBCS can comprise between about 8% v/v and about 12% v/v of the cell culture media. In an example, the mixture of FCS and NBCS can comprise about 10% v/v of the cell culture media However, in this example, the cell culture media is supplemented with at least about 1% v/v, at least about 2% v/v, at least about 3% v/v, at least about 4% v/v, at least about 5% v/v, at least about 6% v/v, at least about 7% v/v, at least about 8% v/v, at least about 9% v/v, but less than 10% v/v FCS. In an example, the cell culture media is supplemented with between about 1% v/v and about 9% v/v FCS. In an example, the cell culture media is supplemented with between about 3% v/v and about 8% v/v FCS. In an example, the cell culture media is supplemented with between about 3% v/v and about 6% v/v FCS. In an example, the cell culture media is supplemented with about 5% v/v FCS.

### Ascorbic Acid

In an example, the cell culture media is supplemented with a short acting ascorbic acid derivative. The term “short acting” encompasses ascorbic acid derivatives that are oxidised by approximately 80-90% following 24 hours of cell culture under culture conditions of neutral pH and 37° C. In one example, the short acting L-ascorbic acid derivative is a L-ascorbic acid salt, for example L-ascorbic acid sodium salt. In an example, the cell culture media may contain at least about 0.005 g/L of a short acting ascorbic acid derivative. In another example, the cell culture media may contain at least about 0.01 g/L of a short acting ascorbic acid derivative. For example, the cell culture media may contain at least about 0.02 g/L of a short acting ascorbic acid derivative. In another example, the cell culture media may contain at least about 0.03 g/L of a short acting ascorbic acid derivative. For example, the cell culture media may contain at least about 0.04 g/L of a short acting ascorbic acid derivative. In another example, the cell culture media may contain at least about 0.05 g/L of a short acting ascorbic acid derivative. In another example, the cell culture media may contain at least about 0.06 g/L of a short acting ascorbic acid derivative.

In another example, the cell culture media contains a short acting ascorbic acid derivative but does not contain a substantial amount of a long acting ascorbic acid derivative. For example, the cell culture media may contain a short acting ascorbic acid derivative but not more than 0.04 g/L of a long acting ascorbic acid derivative. In another example, the cell culture media may contain a short acting ascorbic acid derivative but not more than 0.03 g/L of a long acting ascorbic acid derivative. In another example, the cell culture media may contain a short acting ascorbic acid derivative but not more than 0.02 g/L of a long acting ascorbic acid derivative. In another example, the cell culture media may contain a short acting ascorbic acid derivative but not more than 0.01 g/L of a long acting ascorbic acid derivative. In another example, the cell culture media may contain a short acting ascorbic acid derivative but not more than 0.005 g/L of a long acting ascorbic acid derivative. In another example, the cell culture media may contain a short acting ascorbic acid derivative but not a long acting ascorbic acid derivative. In another example, the cell culture media contains L-ascorbate sodium salt but does not contain a substantial amount of L-ascorbic acid-2-phosphate.

### Other Additives

In an example, the cell culture medium contains human derived additives. For example, human serum and human platelet cell lysate can be added to the cell culture media. In other examples, additional factors can be added to the cell culture medium. For example, the cell culture media can be supplemented with one or more stimulatory factors selected from the group consisting of, platelet derived growth factor (PDGF), fibroblast growth factor 2 (FGF2), epidermal growth factor (EGF), epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof.

In the above examples, basal medium such as Alpha MEM or StemSpan™ can be supplemented with the referenced quantity of serum and, in certain examples, other additives. Further examples of suitable culture mediums for culturing stem cells can be found, for example, in WO2016139340.

### Angiogenic Markers

According to the present disclosure, in certain embodiments, MLPSCs cultured according to the methods disclosed herein express increased levels of one or more angiogenic markers. The present inventors have surprisingly identified that MLPSCs expressing increased levels of one or more angiogenic markers have improved therapeutic efficacy in patients with progressive heart failure. Accordingly, in an example, methods of the disclosure relate to selection of culture expanded MLPSCs for use in treatments such as treatment of progressive heart failure. Such methods comprise determining the level(s) of a marker(s) disclosed herein and, selecting for use in treatment MLPSCs that have increased levels of one or more of the marker(s).

Angiogenesis is the physiological process through which new blood vessels form. In pathophysiological events such as ischemia and inflammation, angiogenesis is increased at the site of injury due to the release of growth factors such as vascular endothelial growth factor (VEGF) and chemokines such as stromal cell-derived factor 1 (SDF-1). SDF-1α is a pro-angiogenic protein that is known to play role in the migration, recruitment, and retention of endothelial progenitor cells to sites of ischemic injury and contributes to neovascularization. VEGF is considered the most important regulator of blood vessel formation in health and disease and is essential for embryonic vasculogenesis, angiogenesis, as well as being a key mediator of neovascularization in cancer and other diseases. VEGF acts through a family of cognate receptor kinases in endothelial cells to stimulate blood-vessel formation. At the cellular level, VEGF binding to its main receptor kinase-insert-domain-containing receptor (KDR) imitates a complex network of signalling pathways including activation of phospholipase C-gamma, protein kinase C, Ca(2+), ERK (extracellular-signal-regulated protein kinase), Akt, Src, focal adhesion kinase and calcineurin pathways.

Angiogenin is another potent pro-angiogenic factor that regulates angiogenesis and cell proliferation by stimulating basement membrane degradation, endothelial cell penetration, migration and formation of tubular vascular structures. Angiogenin induces angiogenesis after binding to actin on the surface of endothelial cells. Angiogenin is a member of the RNase A superfamily and is encoded by the ANG gene in humans (NCBI Gene ID: 283; GenBank: AAH62698.1). The structure, function an expression pattern of angiogenin is known in the art, along with methods for detection (see, for example Tello-Montoliu et al. J Thromb Haemost. 2006; 4(9):1864-74.). A range of commercially available antibodies directed to human angiogenin can be used to detect the protein in fluids such as serum, plasma, cell culture supernatant (for example, cell conditioned media), and urine using commercially available enzyme linked immunoabsorbance assays (ELISA) kits. Antibody-based detection assays can also be used to measure angiogenin in tissue or cell lysates. Other approaches to measure angiogenin employ the use of human cytokine protein array technology, for example, Luminex assays, where antibody arrays can be used to simultaneously detect angiogenin among multiple additional factors from a variety of sources.

Methods of the disclosure involve measuring the level of pro-angiogenic factors, such as VEGF, angiogenin and/or SDF-1α, expressed by MLPSCs under culture conditions. In an example, MLPSCs can be culture expanded in culture media according to the methods disclosed herein. Conditioned media from cultured MLPSCs is then isolated (i.e. a sample is obtained from the cell culture) and the amount of expressed angiogenic marker contained therein is measured. The level of angiogenic markers in MLPSC-conditioned media can be measured by standard protein detection methods and/or gene expression methods known in the art. In an example, the level of angiogenic marker is measured by enzyme-linked immunosorbent assay (ELISA). For example, conditioned media of MLPSCs is obtained and then contacted with anti-VEGF antibody, anti-SDF-1α antibody, and/or an anti-angiogenin antibody. Extent of antibody binding is used to quantify the level of angiogenic marker in the conditioned media (e.g. ng/L). In this example, the level of angiogenic marker in the conditioned media is a measure of the level of angiogenic marker expressed or secreted by MLPSCs.

In an example, the level of angiogenic marker is measured by a Western blot. In an example, the level of angiogenic marker is measured by a Luminex assay. In an example, the level of angiogenic marker is measured by reverse transcription RT-PCR. For example, the level of angiogenin in conditioned media from cultured MLPSCs is measured by a cytokine protein array, such as a Luminex assay.

In an example, MLPSCs are selected for use in treatment if they express elevated levels of vascular endothelial growth factor (VEGF). In an example, level of VEGF is greater than about 3 ng/ml. In an example, the level of VEGF is greater than between about 3 ng/ml and 4 ng/ml. In an example, level of VEGF is greater than about 3.1 ng/ml. In an example, level of VEGF is greater than about 3.2 ng/ml. In an example, level of VEGF is greater than about 3.3 ng/ml. In an example, level of VEGF is greater than about 3.4 ng/ml. In an example, level of VEGF is greater than about 3.5 ng/ml. In an example, the level of VEGF is between about 3 ng/ml and 4 ng/ml. In an example, the level of VEGF is between about 3.2 and 3.6 ng/ml. In an example, the level of VEGF is about 3.45 ng/ml.

In an example, MLPSCs are selected for use in treatment if they have increased levels of VEGF relative to a population of MLPSCs that have been culture expanded in a cell culture medium comprising 10% fetal calf serum. In an example, the level of VEGF is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of VEGF is increased by between about 5% and about 60%. In an example, the level of VEGF is increased by between about 5% and about 40%. In an example, the level of VEGF is increased by about 40%. In an example, the level of VEGF is increased by at least about 5%. In an example, the level of VEGF is increased by at least about 10%.

In an example, MLPSCs are selected for use in treatment if they express elevated levels of angiogenin. In an example, the level of angiogenin is greater than about 1000 pg/ml. In an example, the level of angiogenin is greater than about 1100 pg/ml. In an example, the level of angiogenin is between about 1000 pg/ml and 1200 pg/ml. In an example, the level of angiogenin is between about 1100 pg/ml and 1150 pg/ml. In an example, the level of angiogenin is about 1114 pg/ml or higher. In an example, the level of angiogenin is greater than about 1200 pg/ml.

In an example, MLPSCs are selected for use in treatment if they have increased levels of angiogenin relative to a population of MLPSCs that have been culture expanded in a cell culture medium comprising 10% fetal calf serum. In an example, the level of angiogenin is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of angiogenin is increased by between about 5% and about 60%. In an example, the level of angiogenin is increased by between about 5% and about 40%. In an example, the level of angiogenin is increased by about 40%. In an example, the level of angiogenin is increased by at least about 5%. In an example, the level of angiogenin is increased by at least about 10%.

In an example, MLPSCs are selected for use in treatment if they express elevated levels of stromal derived factor 1α (SDF-1α). In an example, the level of SDF-1α is greater than about 3000 ng/ml. In an example, the level of SDF-1α is greater than about 3100 ng/ml. In an example, the level of SDF-1α is greater than about 3200 ng/ml. In an example, the level of SDF-1α is greater than about 3300 ng/ml. In an example, the level of SDF-1α is greater than about 3400 ng/ml. In an example, the level of SDF-1α is greater than about 3500 ng/ml. In an example, the level of SDF-1α is between about 3000 ng/ml and 3500 ng/ml. In an example, the level of SDF-1α is between about 3000 ng/ml and 3400 ng/ml. In an example, the level of SDF-1α is between about 3000 ng/ml and 3300 ng/ml. In an example, the level of SDF-1α is between about 3100 ng/ml and 3400 ng/ml. In an example, the level of SDF-1α is between about 3100 ng/ml and 3300 ng/ml.

In an example, MLPSCs are selected for use in treatment if they have increased levels of SDF-1α relative to a population of MLPSCs that have been culture expanded in a cell culture medium comprising 10% fetal calf serum. In an example, the level of SDF-1α is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of SDF-1α is increased by between about 5% and about 60%. In an example, the level of SDF-1α is increased by between about 5% and about 40%. In an example, the level of SDF-1α is increased by about 40%. In an example, the level of SDF-1α is increased by at least about 5%. In an example, the level of SDF-1α is increased by at least about 10%.

In another example, the angiogenic marker is increased angiogenesis. In this example, increased angiogenesis is measured by an in-vitro angiogenesis assay, for example, a quantitative live-cell imaging assay. Briefly, an endothelial cell line (e.g. human umbilical vein endothelial cells (HUVECs), human dermal fibroblasts, human saphenous vein endothelial cells (HSaVECs), human coronary artery endothelial cells (HCAECs), human aortic endothelial cells (HAECs), brain microvascular endothelial cells (BMEC), or any combination thereof) are fluorescently labelled and seeded into a culture plates. The endothelial cells are then simultaneously incubated in the presence or absence of MLPSC-conditioned media and imaged using a live-cell imaging system.

In this example, angiogenesis can be measured by various network morphometric parameters identified and computed by image analysis software as composite of various elements described in Table 1 (Lam et al. Biomaterials 290. (2022) 121826). In an example, the live-cell imaging system is the IncuCyte® Live-Cell Analysis System. Live-cell imaging systems enable the fluorescent identification of cells and visualization of angiogenesis over time by time-lapse image acquisition. Images can be analysed using computer-based image analysis tools. In an example, the image analysis tool is the IncuCyte® Angiogenesis Analysis Software Module. The IncuCyte® Angiogenesis Analysis Software Module measures angiogenic outputs including endothelial network length, endothelial network area and endothelial branch point formation. The skilled person will appreciate that other image analysis applications can used, for example Image J, CellProfiler. Other examples of live imaging in-vitro angiogenesis assays are disclosed, for example, in Lam et al. Biomaterials 290. (2022). 121826.

TABLE 1
Network morphometric parameters

| Element | Definition |
|---|---|
| Node | Pixel having at least three neighbours |
| Junction | Group of nodes forming a bifurcation |
| Master Junction | Junctions linked with at least three |
| | master segments |
| Extremity | Pixel having only one neighbour |
| Segment | Binary line linked with two junctions |
| Master Segment | Binary lines linked with two junctions |
| | that are not exclusive to one branch |
| Branch | Binary line linked with one junction |
| | and one extremity |
| Isolated Element | Binary line connected to two extremities |
| Mesh | Area enclosed by segments and |
| | corresponding junctions |
| Number of Extremities | Number of Extremities in image |
| Number of Nodes | Number of Nodes in image |
| Number of Junctions | Number of Junctions in image |
| Number of Master | Number of Master Junctions in Image |
| Junctions | |
| Number of Master | Number of Master Segments in Image |
| Segments | |
| Total Master | Sum of all measured Segments in image |
| Segment Length | |
| Number of Meshes | Number of meshes in image |
| Total Mesh Area | Sum of mesh areas in image |
| Number of Pieces | Number of segments, isolated elements, |
| | and branches in image |
| Number of Segments | Number of segments in image |
| Number of Branches | Number of branches in image |
| Number of Isolated | Number of Isolated Segments in image |
| Segments | |
| Total Length | Sum of ‘Branch’, ‘Segment’, and |
| | ‘Isolated Segment’ lengths of |
| | all vessels in image |
| Total Branching Length | Sum of all ‘Branch’ and ‘Segment’ |
| | lengths in image |
| Total Segments Length | Sum of all ‘Segment’ lengths in image |
| Total Branches Length | Sum of all ‘Branch’ lengths in image |
| Total Isolated | Sum of all ‘Isolated Element’ |
| Branches Length | lengths in image |
| Branching Interval | Mean distance separating two |
| | branches in image |
| Mesh Index | Mean distance separating two |
| | master junctions in image |
| Mean Mesh Size | Mean of ‘Mesh’ area in image |
| | (unit - pixel) |

In an example, angiogenesis is measured by the level of endothelial network formation, endothelial network length, and/or endothelial branch length. In an example, angiogenic potential is measured by the level of endothelial network formation, endothelial network length, and/or endothelial branch length. In an example, the level of endothelial network formation, endothelial network length, and/or endothelial branch length is measured after treating a population of endothelial cells with conditioned media obtained from the MLPSCs In an example, the level of endothelial network formation, endothelial network length, and/or endothelial branch length is calculated by the IncuCyte® Angiogenesis Analysis Software Module.

In another example, the endothelial network formation, endothelial network length, and/or endothelial branch length can be calculated as a composite of one or more of number of nodes, number of junctions, number of segments, number of meshes, mean mesh size, total mesh area, number of extremities, total branches length, and/or number of branches. As used herein, “network formation” refers to the network area in units of mm²/mm². Further examples of how endothelial network formation endothelial network length, and/or endothelial branch length can be calculated are described, for example, in Lam et al. Biomaterials 290. (2022).

In an example, MLPSCs are selected for use in treatment if they increase the level of one or more of endothelial network formation, endothelial network length, and/or endothelial branch length. In an example, MLPSCSs are selected when endothelial network formation is greater than about 0.1 mm²/mm². In an example, the endothelial network formation is between about 0.1 mm²/mm² and 0.2 mm²/mm². In an example, the endothelial network formation is about 0.12 mm²/mm². In an example, the endothelial network formation is greater than about 0.12 mm²/mm². In an example, the endothelial network length is greater than about 4 mm²/mm². In an example, the endothelial network length is between about 4 mm²/mm² and about 6 mm²/mm². In an example, the endothelial network length is about 5 mm²/mm². In an example, the endothelial network length is greater than about 5 mm²/mm². In an example, the endothelial branch length is greater than about 12 l/mm². In an example, the endothelial branch length is between about 12 l/mm² and about 17 l/mm². In an example, the endothelial branch length is about 15 l/mm². In an example, the endothelial branch length is greater than about 15 l/mm².

In an example, MLPSCs are selected for use in treatment if they increase the level of one or more of endothelial network formation, endothelial network length, and/or endothelial branch length relative to a population of MLPSCs that have been culture expanded in a cell culture medium comprising 10% fetal calf serum. In an example, the level of endothelial network formation is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of endothelial network formation is increased by between about 5% and about 60%. In an example, the level of endothelial network formation is increased by between about 5% and about 40%. In an example, the level of endothelial network formation is increased by about 40%. In an example, the level of endothelial network formation is increased by at least about 5%. In an example, the level of endothelial network formation is increased by at least about 10%.

In an example, MLPSCs are selected when the level of endothelial network length is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of endothelial network length is increased by between about 5% and about 60%. In an example, the level of endothelial network length is increased by between about 5% and about 40%. In an example, the level of endothelial network length is increased by about 40%. In an example, the level of endothelial network length is increased by at least about 5%. In an example, the level of endothelial network length is increased by at least about 10%.

In an example, MLPSCs are selected when the level of endothelial branch length is increased by about 5%, about 10%, about 20%, about 30%, about 40%, about 50%, about 60%, or about 70% relative to a population of MLPSCs that have been culture expanded in a cell culture medium comprising 10% fetal calf serum. In an example, the level of endothelial branch length is increased by between about 5% and about 60%. In an example, the level of endothelial branch length is increased by between about 5% and about 40%. In an example, the level of endothelial branch length is increased by about 40%. In an example, the level of endothelial branch length is increased by at least about 5%. In an example, the level of endothelial branch length is increased by at least about 10%.

### Potency Assay

The present disclosure provides a potency assay for identifying cells and conditioned media with biological activity or therapeutic efficacy. The potency assay is based on one or more angiogenic markers disclosed herein and determining an increase in the same. Accordingly, in an example, the present disclosure relates to a method for determining the potency of a population of culture expanded mesenchymal lineage precursor or stem cells (MLPSCs) wherein the MLPSCs have been culture expanded in a cell culture medium comprising a non-fetal serum. In this example, the method comprises determining the level of one or more angiogenic markers in the population of MLPSCs. In an example, this method is applied to determine potency of a conditioned media.

In an example, the one or more angiogenic markers is selected from the group consisting of the level of VEGF, angiogenin, SDF-1α expressed by the MLPSCs under culture conditions; and/or, the level of endothelial network formation, endothelial network length, endothelial branch length measured after treating a population of endothelial cells with conditioned media obtained from the MLPSCs. In an example, an increased level of one or more angiogenic markers is indicative of biological activity or therapeutic efficacy. In an example, the potency assay is based on determining an increase in the level of VEGF, angiogenin and/or SDF-1α expressed by MLPSCs under culture conditions. In an example, the potency assay is based on determining an increase in the level of at least two of VEGF, angiogenin and SDF-1α expressed by MLPSCs under culture conditions. In another example, the potency assay is based on determining an increase in VEGF, angiogenin and SDF-1α expressed by MLPSCs under culture conditions. In these examples, VEGF, angiogenin and SDF-1α may be determined via ELISA or via a Luminex assay. In another example, the potency assay is based on determining an increase in endothelial network formation, endothelial network length and/or endothelial branch length, measured after treating a population of endothelial cells with conditioned media obtained from a population of MLPSCs. In an example, the potency assay is based on determining an increase in at least two of endothelial network formation, endothelial network length and endothelial branch length, measured after treating a population of endothelial cells with conditioned media obtained from a population of MLPSCs. In another example, the potency assay is based on determining an increase in endothelial network formation, endothelial network length and endothelial branch length, measured after treating a population of endothelial cells with conditioned media obtained from a population of MLPSCs.

### Compositions

MLPSCs disclosed herein can be culture expanded from a cryopreserved intermediate to produce a preparation containing at least one therapeutic dose.

In an example, compositions of the disclosure comprise around 150 million cells.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of MLPSCs can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11NaO7); 368 mg of sodium acetate trihydrate, USP (C2H3NaO2·3H2O); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotect or, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methylcellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the MLPSCs resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of MLPSCs and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). The exact amount of cells to be administered is dependent upon a variety of factors, including the age, weight, and sex of the subject, and the extent and severity of the disorder being treated.

Despite the number of cells provided in the composition, in an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the MLPSCs comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the composition may optionally be packaged in a suitable container with written instructions for a desired purpose.

Compositions of the disclosure may be administered systemically, such as, for example, by intravenous administration. In an example, compositions are administered transendocardially.

In an example, compositions of the disclosure comprise a “clinically proven effective” amount of MLPSCs. In an example, compositions of the disclosure comprise a “clinically proven effective” amount of MSCs. In an example, compositions of the disclosure comprise a “clinically proven effective” amount of MPCs.

In an example, the “clinically proven effective” amount of MLPSCs is administered as a total dose. The term “total dose” is used in the context of the present disclosure to refer to the total number of cells received by the subject treated according to the present disclosure. In an example, the total dose consists of one administration of cells. In another example, the total dose consists of two administrations of cells. In another example, the total dose consists of three administrations of cells. In another example, the total dose consists of four or more administrations of cells. For example, the total dose can consist of two to four administrations of cells.

### Drug Product and Methods of Manufacturing the Same

The present inventors have also surprisingly identified that MLPSCs which have been cultured in a culture medium according to the methods disclosed herein have/express increased levels of angiogenic markers. MLPSCs expressing increased levels of angiogenic markers, such as angiogenin, promote increased angiogenesis as determined by increased endothelial network formation, endothelial network length, or endothelial branch length. Thus, the present inventors have arrived at a novel population of culture expanded MLPSCs and a conditioned media that can be selected based on high angiogenic potential.

As used herein, “high angiogenic potential” refers to MLPSCs and conditioned media obtained from the same that increase angiogenesis. As described above, increased angiogenesis can be determined by an increase in one or more of endothelial network formation, endothelial network length, or endothelial branch length. As identified by the present inventors, MLPSCs that increase angiogenesis express a particular level of one or more angiogenic markers. Accordingly, in an example, MLPSCs that have high angiogenic potential according to the present disclosure express a particular level of angiogenin measured under culture conditions, and/or induce one or more of endothelial network formation, endothelial network length, or endothelial branch length measured after treating a population of endothelial cells with conditioned media obtained from the MLPSCs. In an example, MLPSCs and/or conditioned media obtained from the same having high angiogenic potential have improved therapeutic efficacy and/or biological activity.

The level of angiogenic marker/s can be measured according to the methods disclosed herein. For example, the level of angiogenin in MLPSC-conditioned media can be measured by standard protein detection methods and/or gene expression methods known in the art. In an example, the level of angiogenin is measured by enzyme-linked immunosorbent assay (ELISA). In an example, the level of angiogenin is measured by a Luminex assay. Endothelial network formation, endothelial network length, and/or, endothelial branch length can be measured in an in-vitro angiogenesis assay as described above.

In an example, the disclosure provides a culture expanded population of MLPSCs, wherein the population of MLPSCs are selected based on high angiogenic potential as determined by the level of angiogenin expressed by the MLPSCs under culture conditions. In an example, the MLPSCs have been culture expanded in a cell culture medium supplemented with at least one pro-inflammatory cytokine disclosed herein and/or a non-fetal serum such as newborn calf serum.

In another example, the disclosure provides a culture expanded population of MLPSCs, wherein the population of MLPSCs are selected based on high angiogenic potential as determined by the level of one or more of the following measured after treating a population of endothelial cells with conditioned media obtained from the MLPSCs: endothelial network formation; endothelial network length; or, endothelial branch length. In an example, the MLPSCs have been culture expanded in a cell culture medium comprising at least one pro-inflammatory cytokine.

In another example, the disclosure provides a conditioned media selected based on high angiogenic potential as determined by the level of one or more of the following measured after treating a population of endothelial cells with the conditioned media: endothelial network formation; endothelial network length; or, endothelial branch length. In an example, the conditioned media is obtained by culture expanding a population of MLPSCs according to the methods disclosed herein.

In an example, the culture expanded population of MLPSCs and/or conditioned media that has been selected based on high angiogenic potential is selected for administration. In an example, such populations or conditioned media can be referred to as a pharmaceutical composition or drug product (DP). In an example, drug product comprises a composition disclosed herein. In an example, the drug product comprises MLPSCs. For example, the DP can comprise 2×10⁶ MLPSCs. In an example, the culture expanded population is an intermediate population disclosed herein.

The present inventors have further identified a method for manufacturing drug product by selecting a population of MLPSCs having high angiogenic potential. Accordingly, in an example, the disclosure provides a method of manufacturing drug product which comprises a population of MLPSCs, the method comprising: acquiring a determination of whether a test population of MLPSCs have a predetermined level of angiogenic potential under culture conditions, and processing at least a portion of the test population of MLPSCs as a drug product if the test population of MLPSCs have at least the predetermined level of angiogenic potential under culture conditions, thereby manufacturing the drug product; or discarding at least a portion of the test population of MLPSCs if the population of MLPSCs has less than the predetermined level of angiogenic potential under culture conditions, wherein angiogenic potential is measured by a predetermined level of angiogenin measured under culture conditions.

In an example, the disclosure provides a method of manufacturing drug product which comprises a population of MLPSCs, the method comprising: acquiring a determination of whether a test population of MLPSCs have a predetermined level of angiogenic potential under culture conditions, and processing at least a portion of the test population of MLPSCs as a drug product if the test population of MLPSCs have at least the predetermined level of angiogenic potential under culture conditions, thereby manufacturing the drug product; or discarding at least a portion of the test population of MLPSCs if the population of MLPSCs has less than the predetermined level of angiogenic potential under culture conditions, wherein angiogenic potential is measured by:
a predetermined level of one or more of the following as measured after treating a population of endothelial cells with conditioned media obtained from the MLPSCs:
   endothelial network formation;
   endothelial network length; and/or,
   endothelial branch length measured in an in-vitro angiogenesis assay.

In an example, the test population is obtained from a population of MLPSCs in 3D culture. For example, the MLPSCs can be in a bioreactor culture. In an example, the test population is obtained from cryopreserved population of MLPSCs. In an example, the test population is representative of a larger population of MLPSCs such as multiple cryopreserved populations of MLPSCs. In an example, the multiple cryopreserved populations of MLPSCs have been culture expanded from the same intermediate population of MLPSCs. In an example, the manufacturing method is applied to conditioned media obtained from the MLPSCs.

As used herein, the term “predetermined level” refers to a level of an angiogenic marker that indicates high angiogenic potential. In an example, the predetermined level is a level of angiogenin that indicates high angiogenic potential. In an example, the predetermined level of angiogenin is greater than about 1200 pg/ml. In an example, the predetermined level of angiogenin is an increase relative to control population of MLPSCs that have been culture expanded in a cell culture medium comprising 10% fetal calf serum.

In an example, the predetermined level is a level of endothelial network formation that indicates high angiogenic potential. In an example, the predetermined level of endothelial network formation is greater than about 0.12 mm²/mm². In an example, the predetermined level of endothelial network formation is an increase relative to control population of MLPSCs that have been culture expanded in a cell culture medium comprising 10% fetal calf serum.

In an example, the predetermined level is a level of endothelial network length that indicates high angiogenic potential. In an example, the predetermined level of endothelial network length is greater than about 5 mm²/mm². In an example, the predetermined level of endothelial network length is an increase relative to control population of MLPSCs that have been culture expanded in a cell culture medium comprising 10% fetal calf serum.

In an example, the predetermined level is a level of endothelial branch length that indicates high angiogenic potential. In an example, the predetermined level of endothelial branch length is greater than about 15 l/mm². In an example, the predetermined level of endothelial branch length is an increase relative to control population of MLPSCs that have been culture expanded in a cell culture medium comprising 10% fetal calf serum.

In an example, the predetermined level is a level of angiogenin, endothelial network formation, endothelial network length, and/or endothelial branch length that indicates high angiogenic potential, according to the parameters shown in Table A.

In an example, the predetermined level is a clinically proven effective predetermined level. In an example, the level is clinically proven effective in the treatment of heart failure. In another example, the predetermined level is predetermined by a regulatory authority such as the US Food and Drug Administration (FDA). In an example, the predetermined level corresponds with increased survival in patients with heart failure. In an example, the predetermined level is a “reference level of angiogenin”. In an example, the reference level of angiogenin is a level of angiogenin of an FDA approved MLPSC population (e.g., an FDA MLPSC population approved for treatment of heart failure). In an example, the reference level of angiogenin provides the criteria for selecting cell populations according to the present disclosure. In an example, the predetermined level is a reference level of one or more of endothelial network formation, endothelial network length, or endothelial branch length. In an example, the reference level of one or more of endothelial network formation, endothelial network length, or endothelial branch length is a level of one or more of endothelial network formation, endothelial network length, or endothelial branch length of an FDA approved MLPSC population (e.g., an FDA MLPSC population approved for treatment of heart failure). In an example, the reference level of one or more of endothelial network formation, endothelial network length, or endothelial branch length provides the criteria for selecting cell populations according to the present disclosure.

In an example, the present disclosure provides methods of manufacturing MSC drug product, such methods include a first step of providing (e.g., culture expanding (e.g., in small scale or large scale cell culture) or manufacturing) or obtaining (e.g., receiving and/or purchasing from a third party (including a contractually related third party or a non-contractually-related (e.g., an independent) third party) a test MLPSC population (e.g., a sample of a test MLPSC population), a second step of acquiring (e.g., detecting, measuring, receiving, or obtaining) at least one value for an MLPSC parameter listed in Table A for the test MLPSC population, and a third step of processing at least a portion of the test MLPSC population (e.g., processing a portion of a manufacturing lot, culture, or run, an entire manufacturing lot, culture, or run, or multiple manufacturing lots, cultures, or runs) as MLPSC drug product (e.g., in a form or packaging intended for administration as described subsequently herein; optionally cryopreserved) if the at least one value for the test MLPSC population meets a reference criterion shown in Table A for the parameter, thereby manufacturing MSC drug product. In an example, the value(s) comprise parameter number 1. In another example, the value(s) comprise parameter number 2. In another example, the value(s) comprise parameter number 3. In another example, the value(s) comprise parameter number 4. In another example, the value(s) comprise parameter number 1 and 2. In another example, the value(s) comprise parameter number 1 and 3. In another example, the value(s) comprise parameter number 1, 2 and 4.

In an example, such methods comprise a second step which includes acquiring values for any combination of two or more MLPSC parameters listed in Table A, and the third step of such methods includes processing at least a portion of the test MLPSC population as MLPSC drug product if the values for the any combination of two or more MLPSC parameters meet the corresponding reference criterion shown in Table A for the parameters.

TABLE A

| Parameter # | Description | Reference Criterion |
|---|---|---|
| 1 | Angiogenin level under culture | ≥1200 pg/ml; or increased relative to a |
| | conditions | control MLPSC population cultured in a |
| | | culture medium comprising 10% FCS. |
| 2 | Endothelial network formation | ≥0.12 mm2/mm2; or increased relative |
| | measured after treating a population | to a control MLPSC population cultured |
| | of endothelial cells with conditioned | in a culture medium comprising 10% |
| | media obtained from the MLPSCs | FCS. |
| 3 | Endothelial network length | ≥5 mm2/mm2; or increased relative to |
| | measured after treating a population | a control MLPSC population cultured in |
| | of endothelial cells with conditioned | a culture medium comprising 10% FCS. |
| | media obtained from the MLPSCs | |
| 4 | Endothelial branch length measured | ≥15 1/mm2; or increased relative to a |
| | after treating a population of | control MLPSC population cultured in a |
| | endothelial cells with conditioned | culture medium comprising 10% FCS. |
| | media obtained from the MLPSCs | |

Populations of MLPSCs with high angiogenic potential can be culture expanded in a cell culture medium comprising at least one pro-inflammatory cytokine according to the methods disclosed herein. For example, MLPSCs are culture expanded in a cell culture medium comprising a non-fetal serum, for example new born serum such as new born calf serum. In an example, MLPSCs are culture expanded in a culture media comprising about 5% non-fetal serum and about 5% fetal serum. In an example, MLPSCs are culture expanded in a medium comprising less than 10% fetal calf serum. In an example, MLPSCs are culture expanded in a xeno-free culture media supplemented with one or more pro-inflammatory cytokines disclosed herein. As used herein, “xeno-free” refers to a culture medium comprising only human-derived components and does not comprise components from non-human animals. In an example, xeno-free culture media comprises human serum. In an example, xeno-free culture media is serum-free.

### Methods of Treatment

Methods of the present disclosure relate to treating progressive heart failure in a subject, the method comprising administering to the subject a composition comprising a population MLPSCs as disclosed herein and/or conditioned media obtained therefrom. Accordingly, in an example, methods of the disclosure comprise administering culture expanded MLPSCs. In another example, methods of the disclosure comprise administering conditioned media or soluble factors obtained therefrom.

Cardiomyopathy is a disease of the heart muscle that makes it harder for the heart to pump blood to the rest of the body. When the heart is unable to pump sufficiently to maintain blood flow to meet the needs of the body heart failure can occur. Cardiomyopathy can occur after an ischemic or non-ischemic event. One cause of ischemic heart failure is systolic dysfunction following a myocardial infarction (MI). A MI occurs when blood stops flowing properly to a part of the heart. The lack of blood supply results in a localized area of myocardial necrosis referred to as an infarct or infarction. The infarcted heart is unable to pump sufficiently to maintain blood flow to meet the needs of the body leading to multiple pathophysiologic responses and ultimately heart failure. Non-ischemic cardiomyopathy is not related to known coronary artery disease. One example, is dilated cardiomyopathy (DCM) where the heart's ability to pump blood is decreased because the heart's main pumping chamber, the left ventricle, becomes enlarged, dilated and weak.

Once the heart is unable to pump sufficiently to maintain blood flow to meet the needs of the body, a series of compensatory mechanisms are initiated, serving to buffer the fall in cardiac output and assisting to maintain sufficient blood pressure to perfuse the vital organs. As a result, patients with heart failure may not progress for extended periods of time. However, the compensatory mechanisms eventually fail to compensate for the damaged heart, resulting in a progressive decline in cardiac output, termed “progressive heart failure”. In the context of the present disclosure, the terms chronic heart failure, congestive heart failure, congestive cardiac failure, systolic dysfunction and advanced heart failure can be used interchangeably with “progressive heart failure”.

The methods of the present disclosure can be used to treat progressive heart failure in a specific populations of MI subjects. Subjects in need of treatment include those already having progressive heart failure as well as those in which progressive heart failure is to be prevented, delayed or halted. In these examples, the subject can have Class II or Class III progressive heart failure based on the NYHA. For example, the subject can have Class II progressive heart failure.

In a first example, the present disclosure relates to the treatment of subjects, defined based on the New York Heart Association (NYHA) classification scale. In an example, the subject's progressive heart failure is less than Class III. In an example, the subject has Class II heart failure. In an example, the NYHA classification is assigned based on the subject's symptoms. For example, the NYHA classification can be assigned based on the following Table:

| Class | Patient Symptoms |
|---|---|
| I | No limitation of physical activity. Ordinary physical |
| | activity does not cause undue fatigue, palpitation, |
| | dyspnea (shortness of breath). |
| II | Slight limitation of physical activity. Comfortable |
| | at rest. Ordinary physical activity results in |
| | fatigue, palpitation, dyspnea (shortness of breath). |
| III | Marked limitation of physical activity. Comfortable |
| | at rest. Less than ordinary activity causes fatigue, |
| | palpitation, or dyspnea. |
| IV | Unable to carry on any physical activity without |
| | discomfort. Symptoms of heart failure at rest. |
| | If any physical activity is undertaken, |
| | discomfort increases. |

In an example, the subject's heart failure results from an ischemic event. In an example, the subject's heart failure results from a myocardial infarction (MI). For example, the subject can be a MI subject. The term “myocardial infarction (MI) subject” is used to define subjects who have had a myocardial infarction. In an example, the subject's heart failure results from a non-ischemic cardiomyopathy.

In an example, the present disclosure relates to the treatment of subjects with progressive heart failure and persistent inflammation. “Persistent inflammation” is defined by elevated C-reactive protein levels. In an example, persistent inflammation is characterised by CRP levels ≥2 mg/L. Accordingly, in this example, the present disclosure relates to the treatment of subjects with progressive heart failure and CRP levels ≥2 mg/L. In an example, these subjects can have Class II or Class III progressive heart failure based on the NYHA. In another example, these subjects have micro-vascular disease and/or macro-vascular disease. For example, these subject can have ischemia and/or diabetes. Accordingly, in an example, the subject can have progressive heart failure, CRP levels ≥2 mg/L, Class II or Class III progressive heart failure and micro-vascular disease and/or macro-vascular disease. In another example, the subject can have progressive heart failure, CRP levels ≥2 mg/L, Class II or Class III progressive heart failure and ischemia and/or diabetes. In another example, the subject can have progressive heart failure, CRP levels ≥2 mg/L, Class II or Class III progressive heart failure and ischemia. In another example, the subject can have progressive heart failure, CRP levels ≥2 mg/L, Class II or Class III progressive heart failure and diabetes. In another example, the subject can have progressive heart failure, CRP levels ≥2 mg/L, Class II progressive heart failure and ischemia. In another example, the subject can have progressive heart failure, CRP levels ≥2 mg/L, Class II progressive heart failure and diabetes.

In a third example, the present disclosure relates to the treatment of subjects with progressive heart failure and micro-vascular disease and/or macro-vascular disease. “Microvascular disease” (sometimes called small artery disease or small vessel disease) is heart disease that affects the walls and inner lining of tiny coronary artery blood vessels that branch off from the larger coronary arteries. In coronary MVD, the heart's coronary artery blood vessels may not necessarily have plaque, but may rather have damage to the inner walls of the blood vessels which can lead to spasms and decreased blood flow to the heart muscle. In an example, the microvascular disease is “myocardial ischemia”, a condition characterised by obstructed blood flow to the heart muscle (myocardium) due to a partial or complete blockage of a coronary artery. Examples of myocardial ischemia include ischemic heart failure, angina and stroke. “Macrovascular disease” is characterised by the process of atherosclerosis, which leads to narrowing of arterial walls in the coronary vascular system. Atherosclerosis is thought to result from chronic inflammation and injury to the arterial wall(s) in the coronary vascular system. By driving inflammation and slowing blood flow, diabetes dramatically accelerates atherosclerosis and therefore represents one example of macrovascular disease. In an example, the diabetes is type I or type II diabetes. In an example, the diabetes is type II diabetes.

Accordingly, in an example, the subject can have progressive heart failure and micro-vascular disease and/or macro-vascular disease. In another example, the subject can have progressive heart failure and ischemia and/or diabetes. In another example, the subject can have progressive heart failure and ischemia. In another example, the subject can have progressive heart failure and diabetes. In these examples, the subject may also have persistent inflammation. For example, the subject can have progressive heart failure, CRP levels ≥2 mg/L and micro-vascular disease and/or macro-vascular disease. In another example, the subject can have progressive heart failure, CRP levels ≥2 mg/L and ischemia and/or diabetes. In another example, the subject can have progressive heart failure, CRP levels ≥2 mg/L and ischemia. In another example, the subject can have progressive heart failure, CRP levels ≥2 mg/L and diabetes.

Subjects from the above referenced first, second and third examples can be further characterised as follows. In an example, subjects treated according to the present disclosure have an initial CRP level ≥2 mg/L. For example, the subject can have Class II or Class III heart failure and an initial CRP level ≥2 mg/L. In another example, the subject can have Class II heart failure and an initial CRP level ≥2 mg/L. In an example, subjects treated according to the present disclosure have an initial CRP level <5 mg/L. In another example, subjects have an initial CRP level <4 mg/L. In another example, subjects have an initial CRP level between 2 and 6 mg/L. In another example, subjects have an initial CRP level between 3 and 6 mg/L. In another example, subjects have an initial CRP level between 4 and 5 mg/L.

There are various assays available for measuring CRP levels such as antibody based immunoassays. For example, CRP levels can be measured in blood samples using an Enzyme-Linked Immunosorbent (ELISA) assay. In an example, a blood sample is obtained from a patient and then purified before being contacted with anti-CRP antibody. Extent of antibody binding is used to quantify the level of CRP in the blood sample (e.g. mg/L).

B-type natriuretic peptide (BNP) is a hormone produced by the heart. N-terminal (NT)-pro hormone BNP (NT-proBNP) is a non-active prohormone that is released from the same molecule that produces BNP. Both BNP and NT-proBNP are released in response to changes in pressure inside the heart. These changes can be related to heart failure and other cardiac problems. Levels goes up when heart failure develops or gets worse, and levels goes down when heart failure is stable. Accordingly, BNP is an effective marker of heart failure progression. In an example, the subject's level of NT-proBNP is less than 2500 pg/ml prior to administering a composition of the disclosure. In another example, the subject's level of NT-proBNP is less than 2400 pg/ml prior to administering a composition of the disclosure. In another example, the subject's level of NT-proBNP is less than 2000 pg/ml prior to administering a composition of the disclosure. In another example, the subject's level of NT-proBNP is less than 1900 pg/ml prior to administering a composition of the disclosure. In another example, the subject's level of NT-proBNP is between 2200 pg/ml and 1000 pg/ml prior to administering a composition of the disclosure. In another example, the subject's level of NT-proBNP is between 2200 pg/ml and 1100 pg/ml prior to administering a composition of the disclosure. In another example, the subject's level of NT-proBNP is between 2100 pg/ml and 1200 pg/ml prior to administering a composition of the disclosure. In another example, the subject's level of NT-proBNP is between 2000 pg/ml and 1500 pg/ml prior to administering a composition of the disclosure.

In an example, the subjects CRP level is >2 mg/ml and their NT-proBNP is >1000 ng/ml.

In another example, the subject has had a heart failure hospitalisation event over the previous 12 months prior to administration of a composition disclosed herein. In another example, the subject has had a heart failure hospitalisation event over the previous 9 months prior to administration of a composition disclosed herein. In another example, the subject has had a heart failure hospitalisation event over the previous 6 to 12 months prior to administration of a composition disclosed herein. In an example, the heart failure hospitalisation event is worsening signs and symptoms of heart failure. In another example, the heart failure hospitalisation event is an ischaemic event. In another example, the heart failure hospitalisation event is a non-ischemic event.

In another example, the subject is able to walk at least 320 meters in 6 minutes prior to administering a composition of the disclosure. In another example, the subject is able to walk at least 330 meters in 6 minutes prior to administering a composition of the disclosure. In another example, the subject is able to walk at least 340 meters in 6 minutes prior to administering a composition of the disclosure. In another example, the subject is able to walk at least 350 meters in 6 minutes prior to administering a composition of the disclosure.

In an example, subjects can have persistent left ventricular dysfunction. Left ventricular dysfunction is characterised by a decrease in myocardial contractility. A reduction in the left ventricular ejection fraction (LVEF) results when myocardial contractility is decreased within the left ventricle. Thus, LVEF provides one way of determining left ventricular dysfunction. Another parameter of left ventricular function is left ventricular end systolic volume (LVESV), a measurement of the adequacy of cardiac emptying, related to systolic function. Another parameter of left ventricular function is left ventricular end diastolic function (LVEDV), a measurement of the adequacy of ventricular filling in diastole (i.e. the amount of blood in the ventricle immediately prior to systole). LVEF and LVESV are often used together to provide an assessment of left ventricular systolic performance and characterise persistent left ventricular dysfunction.

LVEF, LVESV and LVEDV can be measured by a number of methods known in the art such as echocardiogram (e.g. two dimensional echocardiogram), Single Photon Emission Computed Tomography (SPECT), cardio magnetic resonance imaging (cMRI) or multi-gated acquisition scan.

In an example, a subject having an LVEF of less than 45% has left ventricular dysfunction. In other examples, a subject with a LVEF of less than about 44%, 43%, 42%, 41% has left ventricular dysfunction. In another example, a subject with a LVEF of less than about 40% has left ventricular dysfunction. In other examples, a subject with a LVEF of less than about 39%, 38%, 37%, 36%, 35%, 34%, 33%, 32%, 31%, 30% has left ventricular dysfunction.

In the context of the present disclosure the term “persistent left ventricular dysfunction” is used to define left ventricular dysfunction that persists over a period of time or series of measurements. For example, “persistent left ventricular dysfunction” can include left ventricular dysfunction that persists for between about 1 to about 14 days or longer.

In an example, the subject has a LVEF of less than 45%. In another example, the subject has a LVEF of less than 40%. In other examples, the subject has a LVEF of less than 39%, 38%, 37%, 36%, 35%, 34%, 33%, 32%, 31%, 30%.

In an example, the subject has a LVESV greater than 70 ml. In another example, the subject has a LVESV greater than 100 ml. In another example, the subject has a LVESV greater than 130 ml. In another example, the subject has a LVESV between 70 ml and 160 ml. In these examples, the subject can also have an above referenced LVEF. For example, a subject can have a LVESV greater than 70 ml and a LVEF <45%.

In an example, the subject's heart failure results from an ischaemic event or from a non-ischaemic event. In an example, the subject's heart failure results from an ischaemic event disclosed below.

The methods of the present disclosure relate to the treatment of the progressive decline in cardiac output characteristic of progressive heart failure. Accordingly, “treat” and “treatment”, in the context of the present disclosure refers to both therapeutic treatment and prophylactic or preventative measures.

In an example, treatment includes administering a composition of the disclosure. In an example, methods of the present disclosure reduce or inhibit progression of progressive heart failure. In an example, treatment improves the subject's left ventricular function. In example, treatment improves the subject's LVEF. In example, treatment improves the subject's LVEF by at least 1 percentage point (i.e. a subject with an LVEF of 35% prior to treatment improves to an LVEF of 36% after treatment. In an example, treatment improves the subject's LVEF by between 2 and 10 percentage points. In an example, treatment improves the subject's LVEF by between 4 and 7 percentage points. In an example, treatment improves the subject's LVEF by between 5 and 7 percentage points.

In an example, treatment improves a subject's LVESV. In an example, wherein treatment improves the subjects LVEDV by at least 15 ml. In an example, treatment improves the subjects LVESV by at least 17 ml. In an example, treatment improves the subjects LVESV by at least 20 ml. In an example, treatment improves the subjects LVESV by between 15 ml and 30 ml. In an example, treatment improves the subjects LVESV by between 15 ml and 30 ml.

In an example, treatment improves the subject's LVEDV. In an example, treatment improves the subject's LVEDV by at least 15 ml. In an example, treatment improves the subjects LVEDV by between 15 ml and 25 ml.

In an example, treatment inhibits the subject's progression to NYHA class III progressive heart failure. In another example, treatment reduces the risk of cardiac death. In an example, the reduced risk of cardiac death is relative to risk of cardiac death in a subject with NYHA class III progressive heart failure. In an example, the reduced risk of cardiac death is relative to risk of cardiac death in a subject that has not been administered MLPSCs. For example, the reduced risk of cardiac death is relative to risk of cardiac death in a subject with NYHA class III progressive heart failure that has not been administered MLPSCs. In an example, treatment reduces the risk of cardiac death by at least 20%. In an example, treatment reduces the risk of cardiac death by at least 30%. In an example, treatment reduces the risk of cardiac death by at least 40%. In an example, treatment reduces the risk of cardiac death by at least 50%. In an example, treatment reduces the risk of cardiac death by between 35% and 45%. In an example, treatment reduces the risk of cardiac death by between 40% and 45%.

In another example, the risk of ischemic MACE (MI or stroke) is reduced after treatment. In an example, risk of ischemic MACE (MI or stroke) is reduced by at least 50% relative to baseline. In another example, risk of ischemic MACE (MI or stroke) is reduced by at least 55% relative to baseline. In another example, risk of ischemic MACE (MI or stroke) is reduced by at least 60% relative to baseline. In another example, risk of ischemic MACE (MI or stroke) is reduced by at least 65% relative to baseline. In another example, risk of ischemic MACE (MI or stroke) is reduced by at least 70% relative to baseline. In another example, risk of ischemic MACE (MI or stroke) is reduced by at least 50% to 70% relative to baseline.

In another example, the risk of 3-Point MACE (Cardiac death/MI/stroke) is reduced after treatment. In the context of the present disclosure, “3-point MACE” is used to refer to is defined as a composite of cardiovascular death, nonfatal myocardial infarction and nonfatal stroke (Cardiac death/MI/stroke). In an example, risk of 3 point MACE is reduced by at least 30% relative to baseline. In another example, risk of 3 point MACE is reduced by at least 40% relative to baseline. In another example, risk of 3 point MACE is reduced by at least 45% relative to baseline. In another example, risk of 3 point MACE is reduced by at least 50% relative to baseline. In another example, risk of 3 point MACE is reduced by at least 55% relative to baseline. In another example, risk of 3 point MACE is reduced by at least 30% to 50% relative to baseline. In another example, risk of 3 point MACE is reduced by at least 45% to 55% relative to baseline.

In an example, reduced risk is reduced 3 year risk. In another example, the reduced risk is reduced 5 year risk. In these examples, the risk of ischemic event is reduced over a defined period of time.

In an example, treatment increases patient survival. In an example, treatment increases the probability of a subject surviving for at least 1000 days after initiation of treatment. In another example, treatment increases the probability of a subject surviving for at least 2000 days after initiation of treatment. In an example, the increased probability is determined relative to a subject that is not treated with a composition of the disclosure. In an example, the increased probability is determined relative to a subject that has Class III heart failure.

In an example, treatment reduces the chance or risk of heart failure-related Major Adverse Cardiac Events (HF-MACE) defined as a composite of cardiac related death or resuscitated cardiac death, or non-fatal decompensated heart failure events. In an example, the chance or risk of HF-MACE is reduced over at least 6 months, at least 12 months, at least 24 months, at least 36 months after administration of a composition disclosed herein. In an example, treatment reduces the chance or risk of all-cause mortality.

The present inventors have also surprisingly found that heart failure patients who have received a left ventricular assist device (LVAD) and who have heart failure resulting from an ischemic event, also have persistent inflammation. As disclosed herein, MLPSC compositions according to the disclosure are particularly effective for treating heart failure patients with persistent inflammation.

LVADs are mechanical circulatory support devices that can be implanted into patients with end stage heart failure. Patients can have LVAD implantations as a bridge to transplant (BTT) therapy, or as a destination therapy (DT) for subjects ineligible for a transplant. The skilled person would be aware of various LVAD models, including but not limited to HeartMate I, HeartMate II, HeartMate 3, and HeartWare.

Accordingly, in another example, heart failure subjects with persistent inflammation that are treated according to methods of the present disclosure have an LVAD. For example, the subject can have an LVAD and heart failure resulting from an ischemic event. In these examples, subjects with an LVAD and heart failure resulting from an ischemic event have persistent inflammation and this can be characterised, if required, based on the serum level of certain biomarker(s).

For example, persistent inflammation, in the context of an LVAD subject, can be characterised based on plasma IL-6 levels. Accordingly, LVAD patients treated according to the disclosure can have IL-6 levels that are increased relative to baseline. In an example, the subject's IL-6 level is increased relative to baseline at least 30 days after LVAD implantation. In an example, the subject's IL-6 level is increased relative to baseline at least 60 days after LVAD implantation. In an example, the subject's IL-6 level is increased relative to baseline between 30 days and 365 days after LVAD implantation.

In an example, LVAD subjects with heart failure resulting from an ischemic event have an increased risk of all-cause death, relative to a subject who has been implanted with an LVAD and whose heart failure results from a non-ischemic event. As used herein, “all-cause death” (also known as “all-cause mortality”) is the measure of the total number of deaths from any cause. In an example, the subject's risk of all-cause death is about 30% higher than to a subject who has been implanted with an LVAD and whose heart failure results from a non-ischemic event. In an example, treatment of LVAD subjects with MLPSCs according to the present disclosure reduces a subject's risk of all-cause death. In an example, the subject's risk of all-cause death is reduced by between 10% and 90%. In an example, the subject's risk of all-cause death is reduced by greater than about 50%. In an example, the subject's risk of all-cause death is reduced by greater than about 80%. In an example, the subject's risk of all-cause death is reduced by about 80%.

### Ischemic Events

In an example, the present disclosure relates to a method of reducing the risk or incidence of ischemic events in subjects, in particular subjects with cardiomyopathy. In an example, the present disclosure relates to a method of reducing the risk or incidence of ischemic events in subjects with cardiomyopathy and elevated CRP. In an example, risk or incidence is reduced relative to a subject that does not receive a composition of the disclosure. For example, risk or incidence can be reduced relative to an untreated subject. In an example, the ischemic event is caused by the formation of an occlusion. In an example, the occlusion is an arterial occlusion. In an example, the ischemic event is formation of a cerebrovascular occlusion. In another example, the ischemic event is a formation of a cardiac occlusion. For example, the occlusion can form in the coronary artery.

Examples of ischemic events caused by formation of an occlusion include heart attack and stroke. Accordingly, in an example, the present disclosure relates to methods of reducing the risk or incidence of heart attack or stroke in a subject with cardiomyopathy.

The risk or incidence of ischemic events in subjects with cardiomyopathy is reduced by administering a cell therapy such as a composition of the disclosure.

In an example, the ischemic event is non-fatal. In an example, the ischemic event is fatal and, in this example, the method of the disclosure reduces risk of cardiac death from the ischemic event. Accordingly, in an example, the methods of the disclosure encompass a method of reducing risk of cardiac death or a non-fatal ischemic event in a subject, the method comprising administering to the subject a composition comprising MLPSCs. In an example, the subject has one or more or all of:
class II heart failure
micro-vascular disease and/or macro-vascular disease;
persistent inflammation.

For example, the present disclosure encompasses a method of reducing risk of cardiac death or a non-fatal ischemic event in a subject, the method comprising administering to the subject a composition comprising MLPSCs, wherein the subject has class II heart failure.

In another example, the present disclosure encompasses a method of reducing risk of cardiac death or a non-fatal ischemic event in a subject, the method comprising administering to the subject a composition comprising MLPSCs, wherein the subject has micro-vascular disease and/or macro-vascular disease.

In another example, the present disclosure encompasses a method of reducing risk of cardiac death or a non-fatal ischemic event in a subject, the method comprising administering to the subject a composition comprising MLPSCs, wherein the subject has persistent inflammation.

In an example, the subject has non-ischemic cardiomyopathy. For example, the subject's cardiomyopathy may be caused by an enlarged left ventricle (dilated cardiomyopathy. In another example, the cardiomyopathy is caused by a viral infection.

In another example, the subject has Class II or Class III heart failure according to the New York Heart Association (NYHA) classification scale.

In another example, the subject's level of N-terminal pro-B-type natriuretic peptide (NT-proBNP) is between 1000 pg/ml and 2000 pg/ml prior to administering the cells. In another example, the subject's C-reactive protein (CRP) level is elevated. In another example, the subject's CRP level is ≥1.5 mg/L. In another example, the subject's CRP level is ≥2 mg/L. In another example, the subject's CRP level is between 1 and 5 mg/L. In another example, the subject's CRP level is between 3 and 5 mg/L.

In an example, the cells are administered transendocardially.

In an example, reduced risk is reduced 3 year risk. In another example, the reduced risk is reduced 5 year risk. In these examples, the risk of ischemic event is reduced over a defined period of time.

### Selecting a Subject for Treatment

In an example, the methods of the present disclosure relate to methods of selecting a subject with persistent inflammation and/or elevated risk of cardiac death for treatment with stem cell compositions according to the disclosure. In an example, a level of CRP ≥2 mg/L indicates persistent inflammation and elevated risk of cardiac death, myocardial infarction or stroke. In an example, the level of CRP is measured after an ischemic event. In an example, the ischemic event is a myocardial infarction.

Accordingly, in an example, the present disclosure relates to a method of treating progressive heart failure, the method comprising the steps of: i) selecting a subject having a CRP level ≥2 mg/L for treatment, and ii) administering to the subject a composition comprising MLPSCs, wherein the MLPSCs have been culture expanded in a cell culture medium comprising a non-fetal serum and, wherein the subject has persistent inflammation

In another example, the method comprises the steps of: i) selecting a subject having progressive heart failure for treatment, wherein the subject has a micro-vascular disease and/or a macro-vascular disease, and ii) administering to the subject a composition comprising MLPSCs, wherein the MLPSCs have been culture expanded in a cell culture medium comprising a non-fetal serum and, wherein the subject has persistent inflammation.

In another example, the method comprises the steps of: i) selecting a subject having progressive heart failure for treatment, wherein the subject has been implanted with an LVAD, and ii) administering to the subject a composition comprising MLPSCs, wherein the MLPSCs have been culture expanded in a cell culture medium comprising a non-fetal serum and, wherein the subject has persistent inflammation.

In another example, the method comprises the steps of: i) selecting a subject having progressive heart failure for treatment, wherein the subject has CRP level >2 mg/ml and NTpro-BNP level >1000 ng/ml, and ii) administering to the subject a composition comprising MLPSCs, wherein the MLPSCs have been culture expanded in a cell culture medium comprising a non-fetal serum and, wherein the subject has persistent inflammation.

In another example, the method comprises the steps of: i) selecting a subject having progressive heart failure for treatment, wherein the subject has a micro-vascular disease and/or a macro-vascular disease, and ii) administering to the subject conditioned media or a population of exosomes derived therefrom, wherein the conditioned media is obtained by culture expanding a population of MLPSCs in a cell culture medium comprising a non-fetal serum and, wherein the subject has persistent inflammation.

In another example, the method comprises the steps of: i) selecting a subject having progressive heart failure for treatment, wherein the subject has been implanted with an LVAD, and ii) administering to the subject conditioned media or a population of exosomes derived therefrom, wherein the conditioned media is obtained by culture expanding a population of MLPSCs in a cell culture medium comprising a non-fetal serum, wherein the subject has persistent inflammation.

In another example, the method comprises the steps of: i) selecting a subject having progressive heart failure for treatment, wherein the subject has CRP level >2 mg/ml and NTpro-BNP level >1000 ng/ml, and ii) administering to the subject conditioned media or a population of exosomes derived therefrom, wherein the conditioned media is obtained by culture expanding a population of MLPSCs in a cell culture medium comprising a non-fetal serum.

### EXAMPLES

### Example 1: Serum Analysis

Mesenchymal precursor lineage or stem cell populations were culture expanded in either 5% FCS/5% NBCS (serum A) or 10% fetal bovine serum (also known as fetal calf serum) (serum B). These MLPSCs were used in examples 4 to 7.

Cytokine levels in 5% FCS/5% NBCS (serum A) and 10% fetal bovine serum (serum B) were assessed. To provide an external control, cytokine levels were also assessed in FBS from a different supplier (serum C). In each instance, cytokine levels were assessed in neat serum.

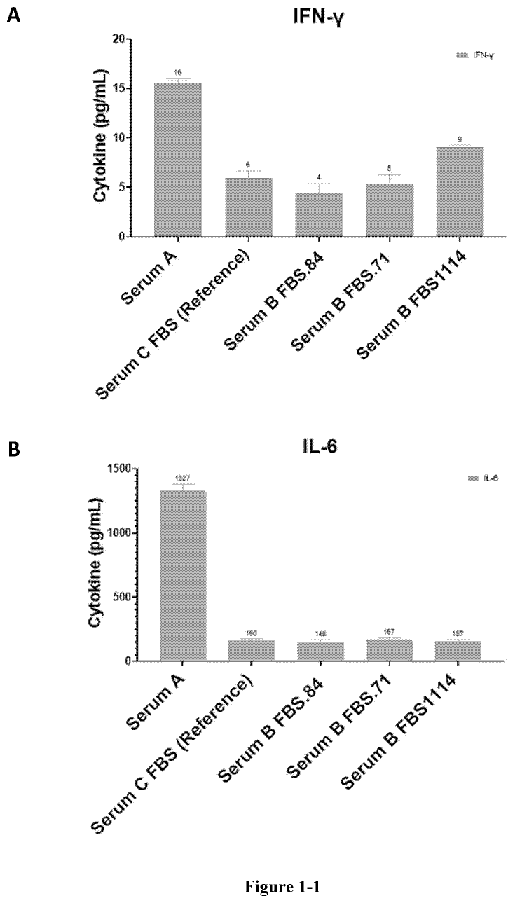

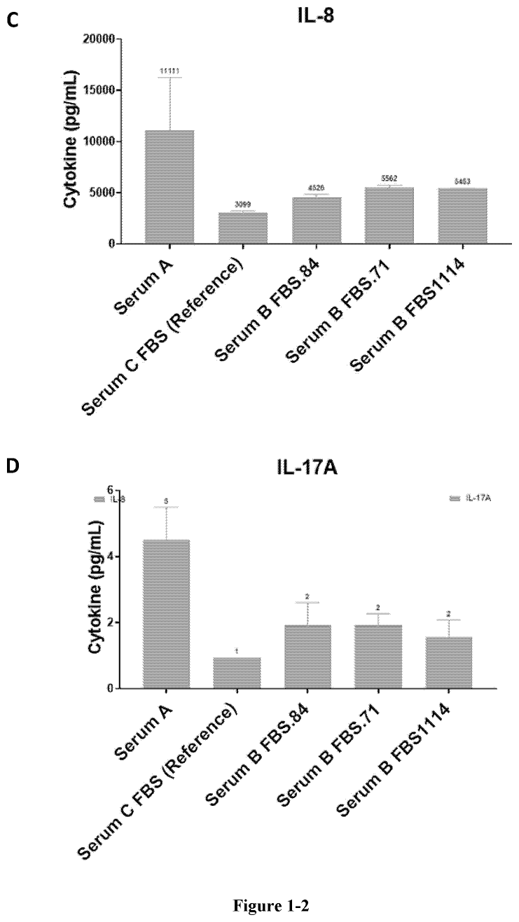

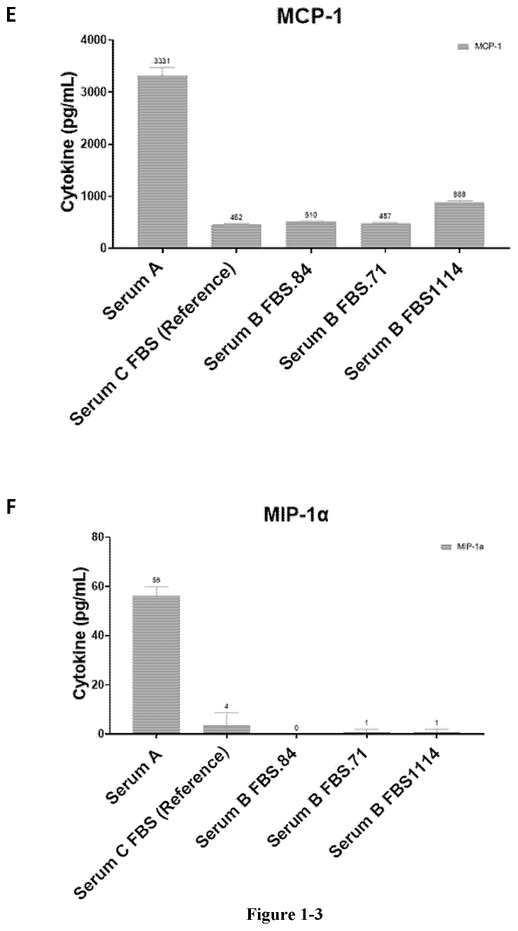

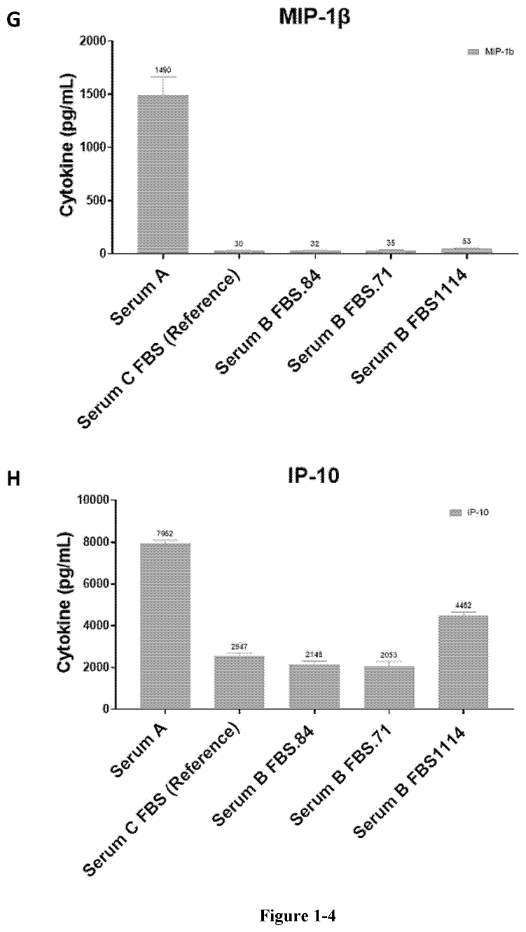

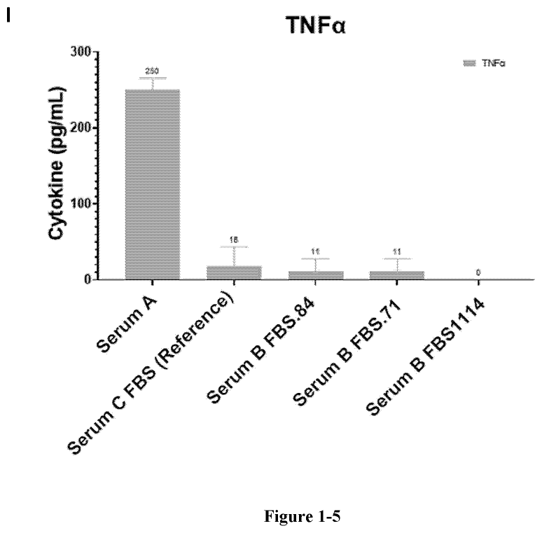

Surprisingly, pro-inflammatory cytokine levels were higher in serum preparations containing newborn calf serum (FIG. 1). Notably, there was an increase in pro-inflammatory cytokines known to bind receptors expressed on the surface of MLPSCs, such as interferon gamma (IFNγ), tumor necrosis factor alpha (TNFα) and, interleukins. For example, the following was observed in serum preparations containing newborn calf serum relative to fetal bovine serums:
At least a 2× increase in IFNγ;
At least a 13× increase in TNFα;
At least an 8× increase in IL-6;
At least a 2× increase in IL-8;
At least a 2× increase in IL-17A.

### Example 2: MLPSC Compositions Derived Using Culture Media Supplemented with Fetal Serum

The Alpha modification of Eagle's minimum essential media (MEM) with Earle's balanced salts, commonly referred to as Eagle's Alpha MEM, contains non-essential amino acids, sodium pyruvate, and additional vitamins. These modifications were first described for use in growing hybrid mouse and hamster cells (Stanners et al. 1971).

Eagle's Alpha MEM media suitable for culturing primary stem cells can be obtained from a variety of sources, including Life Technologies and Sigma.

A detailed method of establishing primary stem cell cultures, including the required growth factors used in the Exemplified processes is described in Gronthos and Simmons 1995.

Eagle's Alpha MEM media supplemented with 10% fetal calf serum (serum B), L-ascorbate-2-phosphate (100 μM), dexamethasone (10⁻⁷ M) and/or inorganic phosphate (3 mM) was used for culturing MLPSCs.

### Example 3: MLPSC Compositions Derived Using Culture Media Comprising Newborn Serum

For the MLPSC culture media comprising newborn serum, the serum component of the Eagle's Alpha MEM culture media described in Example 2 was modified by supplementing with 5% (v/v) newborn serum (Differences in the fetal serum media and newborn serum media are shown in Table 2). The newborn serum used was newborn calf serum (NBCS). NBCS was 100% bovine serum obtained from animals meeting the standard fetal bovine serum specifications but under the age of 20 days after birth.

NBCS was obtained from a commercial supplier, where it is marketed as an FCS substitute that is highly similar to FCS, to be used interchangeably, and expected to perform the same on cell lines.

TABLE 2
Summary of the differences between fetal serum
culture media and licensing culture media

| | |
|---|---|
| Fetal serum culture media | Licensing culture media |

Media (Change applicable to Thaw Feed, Passage)

| | |
|---|---|
| Alpha MEM | 50 mg/L short acting ascorbic acid derivative (Sodium L- |
| 10% v/v FCS | ascorbate) replaces long acting ascorbic acid derivative |
| | (L-ascorbic acid-2-phosphate) |
| | 5% v/v FCS and 5% v/v newborn serum |

Cryopreservation Formulation
(50% Alpha-MEM/42.5% ProFreeze/7.5% DMSO)

| | |
|---|---|
| Alpha MEM | 50 mg/L short acting ascorbic acid derivative (Sodium L- |
| 10% v/v FCS | ascorbate) replaces long acting ascorbic acid derivative |
| | (L-ascorbic acid-2-phosphate) |
| | 5% v/v FCS |
| | 5% v/v newborn calf serum |

### Example 4: Culture Expansion of MLPSCs in Media Supplemented with Newborn Serum Enhances Angiogenesis

With a view to characterising the novel MLPSC populations derived by culture expansion in media supplemented with newborn serum and/or pro-inflammatory cytokines (and to potentially identify a mechanism for the observed increase in therapeutic efficacy described in Example 5), we assessed the angiogenic potential of MPCs cultured under different conditions.

Cell culture: MPCs were cultured with either 5% NBCS/5% FCS (serum A) or 10% FCS (serum B) to generate MPC-conditioned media. To control for donor variation, MPCs were obtained from the same donor and then cultured under different conditions. In some experiments, MPCs belonging to same donor but cultured during a different manufacturing expansion are indicated by separate “lot” numbers.

Conditioned media was obtained by separating the cells from the culture media supernatant. Briefly, cryopreserved MPCs were thawed and seeded at 50,000/cm² in alpha MEM and either 10% FBS or 5% NBCS/5% FCS. Conditioned media (CM) was collected after incubating the cells for 72 h at 37° C. 5% CO₂. VEGF, SDF-1 and angiogenin levels in CM were measured using Luminex (R&D Systems). The CM was concentrated using a 3k protein concentration filtration column (Amicon® Ultra-15) and reconstituted back to 1× or 0.25× in Assay medium.

Angiogenesis potency assay: In-vitro angiogenesis was measured using a kinetic, quantitative 96-well co-culture angiogenesis model. Lentivirus-transduced human umbilical vein endothelial cells (HUVEC) expressing CytoLight Green (a GFP variant) cultured with normal human dermal fibroblasts (NHDF) were seeded into 96-well plates and simultaneously incubated and imaged using the IncuCyte® Live-Cell Analysis System. This system enables the fluorescent identification of HUVEC (CytoLight Green⁺) cells and allows visualization of tube formation over time by time-lapse image acquisition. The acquired images are analysed using an integrated angiogenesis algorithm to measure network length, network area and branch point formation to provide a quantitation of the stage and extent of angiogenesis throughout the assay.

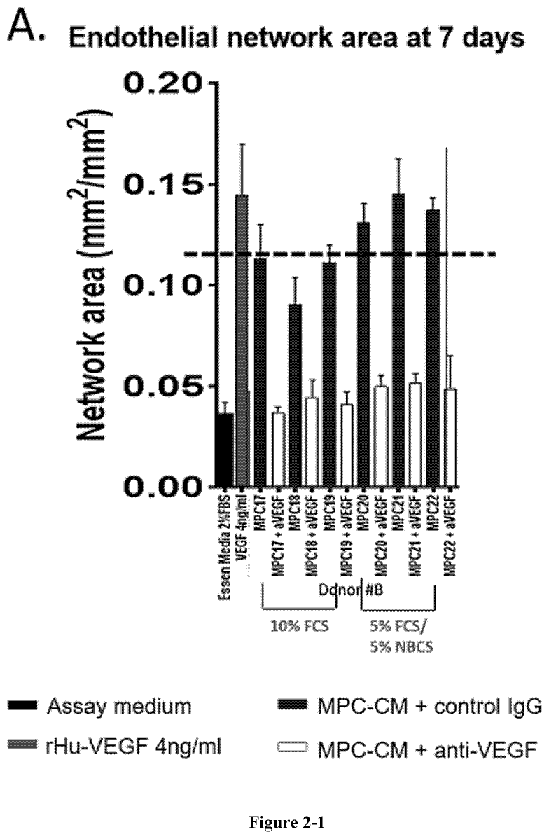

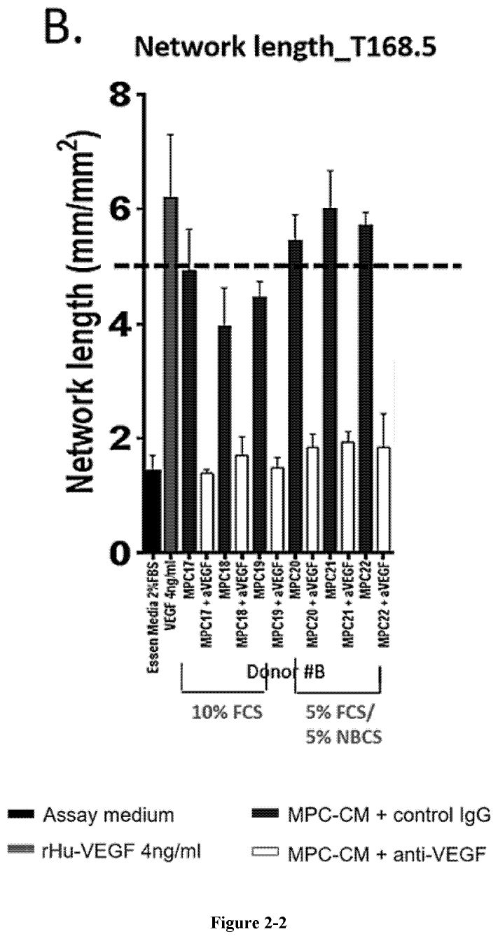

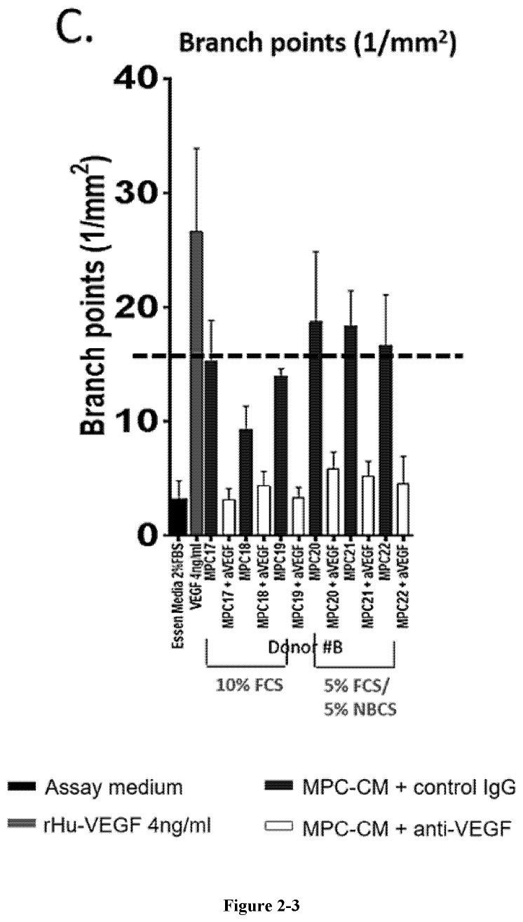

Results: Conditioned media from MPCs cultured in media supplemented with newborn calf serum was found to increase angiogenesis. As shown in FIG. 2, conditioned media from MPCs cultured in 5% NBCS/5% FCS increased network area (FIG. 2A), network length (FIG. 2B) and branch points (FIG. 2C) in the co-culture angiogenesis model. Angiogenin levels were also increased in conditioned media from MPCs cultured in 5% NBCS/5% FCS when compared to 10% FCS (FIG. 3). FIG. 4 shows further analysis of the levels of angiogenic factors SDF-1α, VEGF and Ang1 (ANGPT1) present in MPCs cultured in either 10% FCS (“serum B Media”) or 5% FCS/5% NBCS (“serum A Media”). These data show that both VEGF and SDF-1α are elevated in newborn serum media-cultured MPCs.

In view of the data provided in Example 1, these data indicate that culture expansion of MLPSCs in media supplemented with newborn serum and/or pro-inflammatory cytokines provides novel cell populations with enhanced angiogenic potential. This enhanced potential may be, if required, characterised in various ways (for example, to define the novel cell populations identified by the present inventors) including, for example:
Capacity for conditioned media derived from the MLPSC to increase network area, network length and/or branch points when contacted with HUVECs;
Angiogenin, VEGF and/or SDF-1 levels in conditioned media.

Without wishing to be bound by a particular theory, these data provide a potential mechanism via which MPCs cultured in newborn serum have improved therapeutic efficacy, namely, the enhanced angiogenesis and increased production of pro-angiogenic growth factors, VEGF, SDF-1α and angiogenin. Furthermore, these data provide basis for a method of selecting cells with a sufficient potency for the treatment of inflammatory disorders. In particular, the data shows that a threshold level of about >3.45 ng/mL VEGF, >3000 ng/ml SDF-1α, or >1114 μg/mL angiogenin, with concentrations in advance of these amounts indicating therapeutic potency and increased biological activity of MPCs. For example, cells can be cultured according to the methods disclosed herein, conditioned media could be harvested and measured in the angiogenesis assay and/or for levels of VEGF and angiogenin. Cells which produce VEGF/angiogenin above the threshold are considered therapeutically potent/biologically active. Similarly, cells which produce conditioned media that enhances angiogenesis as determined by a network area of about >0.12 mm²/mm², network length of >5 mm²/mm² and/or branch points of >15 l/mm² are also considered to be therapeutically potent/biologically active for treating diseases with an inflammatory component such as heart failure in patients with persistent inflammation.

### Example 5: Selection of a Population of MPCs with High Angiogenic Potential

Introduction: The results from Example 4 indicate that MPCs with high angiogenic potential can be selected based on (i) the level of growth factors expressed in MPC-conditioned media, and/or (ii) the level of network area, network length and branch length induced in endothelial cells treated with conditioned media obtained from MPCs.

Methods: MPCs were cultured with either 10% FCS, 5% NBCS/5% FCS or xeno-free media to generate MPC-conditioned media (CM). Per Example 4, angiogenin, VEGF and Ang1 levels in CM were measured using Luminex (R&D Systems). Endothelial network area, network length and branch length was measured using the angiogenesis potency assay described in Example 4 above and shown in FIG. 2.

Results: FIG. 3 and Table 3 shows that MPCs cultured in both 5% NBCS/5% FCS and xeno-free media express elevated levels of angiogenin. MPCs cultured in 10% FCS had a mean angiogenin level of approximately 695 pg/ml, whereas MPCs cultured in 5% NBCS/5% FCS and xeno-free media had a mean of angiogenin level of approximately 2010 pg/ml and 1372 pg/ml, respectively (combined mean=1691 pg/ml). This represents an approximate two-fold increase in angiogenin levels relative to MPCs cultured in 10% FCS.

TABLE 3
Angiogenin levels (pg/ml) in MPC conditioned media

| | 10% FCS | 5% FCS/5% NCBS | Xeno-free | |
|---|---|---|---|---|
| Donor A | 1146.99 | 3200.10 | Replicates | 1838.69 |
| | 541.05 | 2238.73 | 1-5 | 1132.15 |
| | 396.19 | 2763.70 | | 1518.46 |
| | 824.95 | | | 1129.78 |
| Donor B | 726.99 | 1257.92 | | 1245.31 |
| | 675.45 | 1490.25 | | |
| | 555.08 | 1114.79 | | |
| Mean | 695.24 | 2010.91 | | 1372.88 |

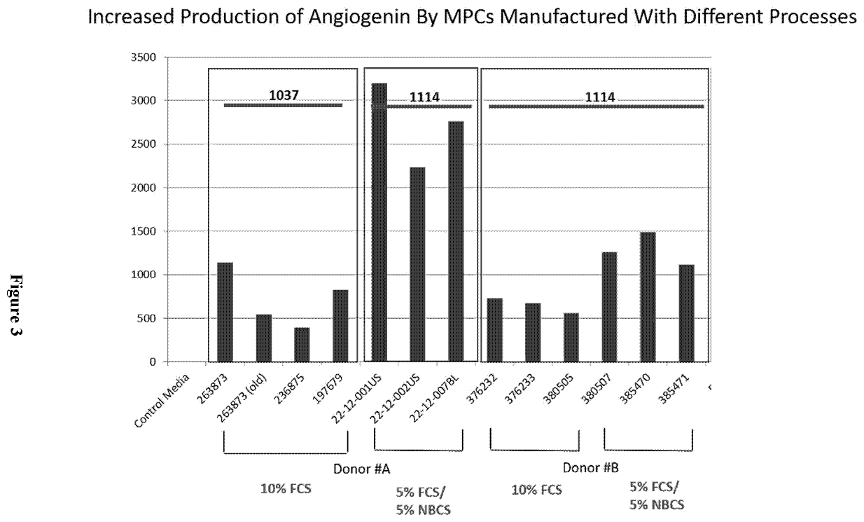

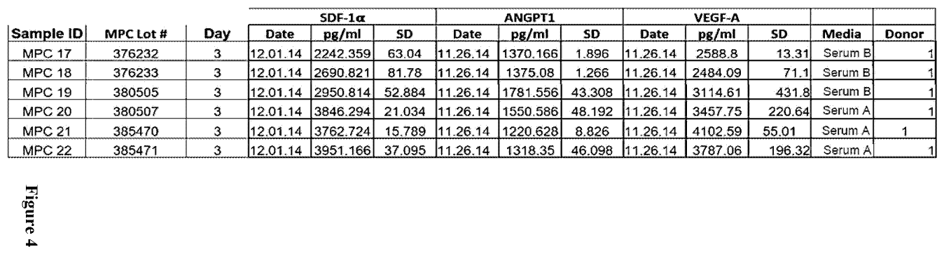

FIG. 2 shows that conditioned media from the same cells expressing high levels of angiogenin also induces increased network area, network length and branch length in endothelial cells. Conditioned media from MPCs with a mean angiogenin level of approximately 2010 pg/ml (cultured in 5% NBCS/5% FCS) increased network formation (as measured by network area) (FIG. 2A), network length (FIG. 2B) and branch points (FIG. 2C), as measured in the angiogenesis potency assay described in Example 4. Network area, network length, and branch points are increased relative to MPCs cultured in 10% FBS (mean angiogenin level of approximately 695 pg/ml). Cells cultured in xeno-free media (mean angiogenin level of approximately 1372 pg/ml) also increased network area (FIG. 6A), network length (FIG. 6B) and branch length (FIG. 6C), relative to MPCs cultured in 10% FCS (mean angiogenin level of approximately 695 pg/ml). The estimated raw values of network area, network length, and branch length are presented in Table 4.

TABLE 4
Network area, network length and branch length in endothelial
cells treated with conditioned media obtained from MPCs

| 10% FCS | | 5% FCS/5% NCBS | | Xeno-free | |
|---|---|---|---|---|---|
| | Network | | Network | | Network |
| | area | | area | | area |
| Sample | (mm²/mm²) | Sample | (mm²/mm²) | Sample | (mm²/mm²) |
| MPC17 | 0.11 | MPC20 | 0.14 | MPC1 | 0.15 |
| (Donor B) | | (Donor B) | | | |
| MPC18 | 0.09 | MPC21 | 0.15 | MPC2 | 0.17 |
| (Donor B) | | (Donor B) | | | |
| MPC19 | 0.11 | MPC22 | 0.14 | MPC3 | 0.16 |
| (Donor B) | | (Donor B) | | | |
| | | | | MPC4 | 0.16 |
| Mean | 0.10 | | 0.14 | | 0.16 |
| network | | | | | |
| area | | | | | |
| 10% FCS | | 5% FCS/5% NCBS | | Xeno-free | |
| | Network | | Network | | Network |
| | length | | length | | length |
| Sample | (mm²/mm²) | Sample | (mm²/mm²) | Sample | (mm²/mm²) |
| MPC17 | 5 | MPC20 | 5.75 | MPC1 | 6.25 |
| (Donor B) | | (Donor B) | | | |
| MPC18 | 4 | MPC21 | 6 | MPC2 | 7 |
| (Donor B) | | (Donor B) | | | |
| MPC19 | 4.5 | MPC22 | 5.75 | MPC3 | 6.5 |
| (Donor B) | | (Donor B) | | | |
| | | | | MPC4 | 6.5 |
| Mean | 4.5 | | 5.83 | | 6.56 |
| network | | | | | |
| length | | | | | |

| 10% FCS | | 5% FCS/5% NCBS | | Xeno-free | |
|---|---|---|---|---|---|
| | Branch | | Branch | | Branch |
| | length | | length | | length |
| Sample | (1/mm²) | Sample | (1/mm²) | Sample | (1/mm²) |
| MPC17 | 15 | MPC20 | 19 | MPC1 | 24 |
| (Donor B) | | (Donor B) | | | |
| MPC18 | 10 | MPC21 | 18 | MPC2 | 29 |
| (Donor B) | | (Donor B) | | | |
| MPC19 | 14 | MPC22 | 16 | MPC3 | 26 |
| (Donor B) | | (Donor B) | | | |
| | | | | MPC4 | 26 |
| Mean | 13 | | 17.67 | | 26.25 |
| branch | | | | | |
| length | | | | | |

Without wishing to be bound by a particular theory, these data show that angiogenin levels can be used to identify an MPC with high angiogenic potential. For example, MPCs expressing angiogenin levels above a threshold level of approximately 1200 pg/ml have high angiogenic potential, which is demonstrated by their ability to induce increased network area, network length, and/or branch length in an angiogenesis assay. Accordingly, the data provide basis for obtaining novel compositions by selecting MPCs based on high angiogenic potential as determined by the level of angiogenin.

These data also provide basis for obtaining novel compositions by selecting MPCs based on high angiogenic potential as determined by the level of one or more of endothelial network formation, endothelial network length, or endothelial branch length, measured after treating a population of endothelial cells with conditioned media obtained from the MPCs. For example, conditioned media taken from MPCs that is able to induce a threshold level of (i) greater than about 0.12 mm²/mm² network formation, (ii) greater than about 5 mm²/mm² network length, and/or (iii) greater than about 15 l/mm² branch length, indicates that those MPCs have high angiogenic potential.

The findings of the present inventors also indicate that cells cultured in non-fetal serum or xeno-free media produce cells with high angiogenic potential (i.e. increased angiogenin, network formation, network length, and/or branch length), relative to cells that have been cultured in media comprising 10% FCS. Given that 10% FCS is the amount of serum typically used in standard cell culture conditions, this suggests that cells cultured in 10% FCS is an appropriate control to determine angiogenic potential against.

Accordingly, the present inventor's findings also provide basis for selecting cells with high angiogenic potential, wherein high angiogenic potential is determined relative to MLPSCs that have been culture expanded in a control medium comprising 10% FCS. Furthermore, the findings of the present inventors underpin broad application of a high-potency population of MPCs, namely a population of MPCs that have been culture expanded in a cell culture medium comprising at least one pro-inflammatory cytokine and selected based on high angiogenic potential.

### Example 6: Assessment of MPCs Cultured in Non-Fetal Versus Fetal Serum: Treatment Efficacy in Context of Persistent Inflammation

NYHA Class II/III high-risk heart failure with reduced ejection fraction (HFrEF) is a clinical model of persistent inflammation. HFrEF patients are characterized by cardiac and systemic inflammation, as determined by the presence of elevated inflammatory biomarkers. MPCs cultured under different serum conditions were administered to HFrEF patients in the clinical study described below.

In HFrEF patients, cardiac macrophages produce high levels of pro-inflammatory cytokines (IL-6, IL-1, TNF-alpha) which cause endothelial dysfunction and cardiomyocyte apoptosis. Plasma C-Reactive Protein (CRP) levels, as determined by a high sensitivity CRP (hsCRP) assay, reflect hepatic production of acute phase reactants in response to the high levels of pro-inflammatory cytokines (IL-6, IL-1 and TNF-alpha) produced by cardiac macrophages. Accordingly, plasma hsCRP levels (<2 mg/L vs >2 mg/L) are representative systemic measurements reflective of low or high intra-cardiac inflammation. In the following study, HFrEF patients were categorized as having persistent inflammation if their plasma hsCRP levels were >2 mg/L.

Study details: Eligible NYHA Class II/III patients were enrolled in the Double-blind, Randomized, Sham-procedure-controlled, Parallel-Group Efficacy and Safety Study of Allogeneic Mesenchymal Precursor Cells (Rexlemestrocel-L) in Chronic Heart Failure Due to LV Systolic Dysfunction (Ischemic or Nonischemic) (DREAM HF-1) trial. HFrEF patients were administered: (1) MPCs cultured in 10% fetal serum (n=37), (2) MPCs cultured in media supplemented with newborn calf serum (5% FCS/5% NBCS; n=153) or, (3) a sham control (i.e., no MPCs; n=241). As evidenced by the serum analysis described in Example 1, cells cultured in media supplemented with newborn serum were effectively cultured in media comprising increased levels of pro-inflammatory cytokines. Cells were administered in a single transendocardial injection. LV systolic function in HFrEF was measured by echocardiogram (ECHO) parameters including left ventricular ejection fraction (LVEF; %), left ventricular end-systolic volume (LVESV; mL), and left ventricular end-diastolic volume (LVEDV; mL) at baseline and 12 months post treatment. Plasma CRP levels were measured to determine baseline levels of inflammation.

Results: MPCs cultured in media supplemented with newborn calf serum (5% FCS/5% NBCS) were found to improve left ventricular (LV) systolic function in HFrEF patients at 12 months. In particular, newborn serum-cultured MPCs significantly increased LVEF and decreased LVESV compared to sham controls (p=0.0398 and 0.0426, respectively) (FIG. 5).

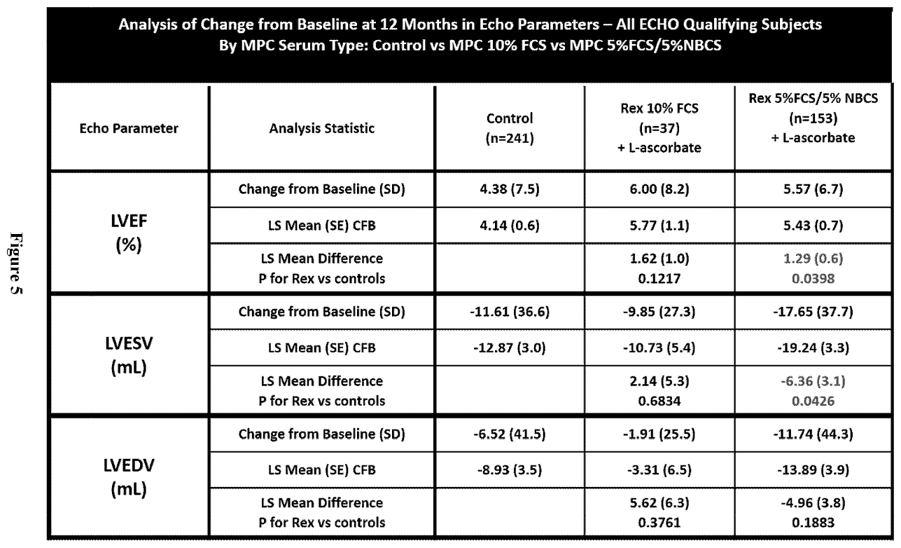

HFrEF patients were then characterised according to plasma hsCRP levels of either <2 mg/L (normal baseline systemic inflammation) or >2 mg/L (elevated baseline systemic inflammation). Importantly, when HFrEF patients were differentiated according to baseline systemic inflammation status (CRP >2), the effect of treatment with MPCs cultured in media supplemented with newborn calf serum (5% FCS/5% NBCS) on LV systolic functional recovery induced was more pronounced. In contrast, MPCs cultured in 10% FBS did not show a significant improvement (FIG. 6). Newborn serum-cultured MPCs significantly increased LVEF % by an average (LS mean) of 2.46 and decreased LVESV by an average of 8.99 mL compared to sham controls (p=0.0033 and 0.0264, respectively). Effect of MPCs cultured in 10% fetal calf serum or 5%/FCS/5% NBCS on LV systolic function in HFrEF patients without elevated baseline inflammation (HFrEF patients with CRP <2) are shown in FIG. 7.

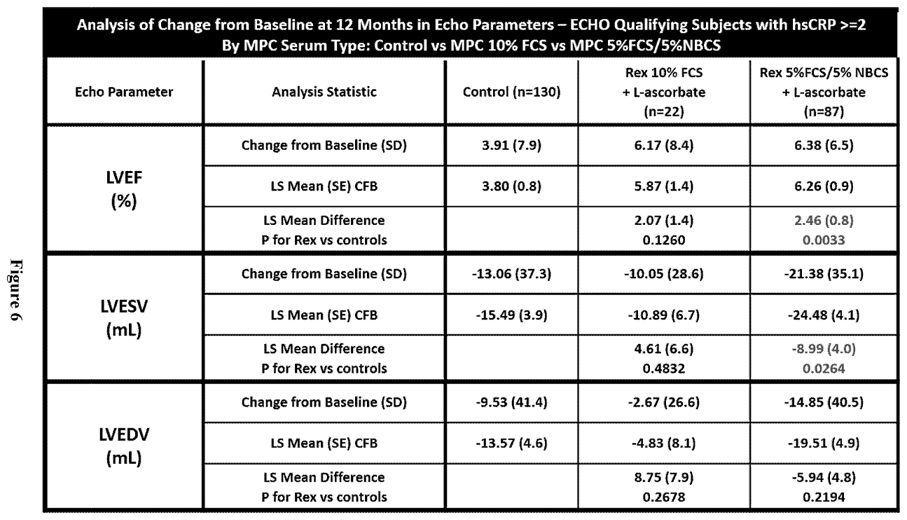

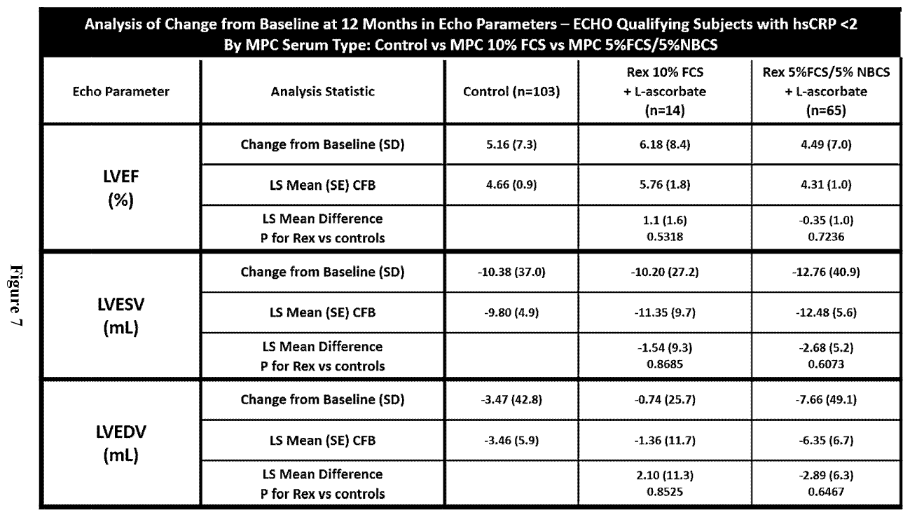

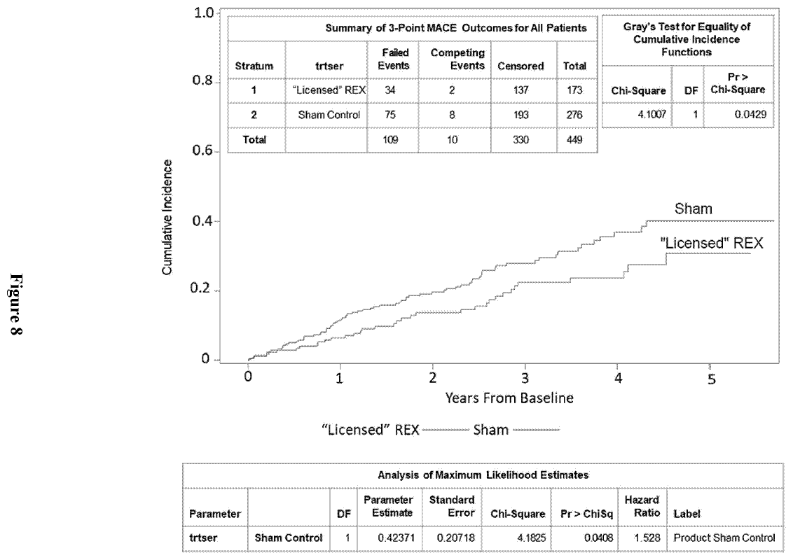

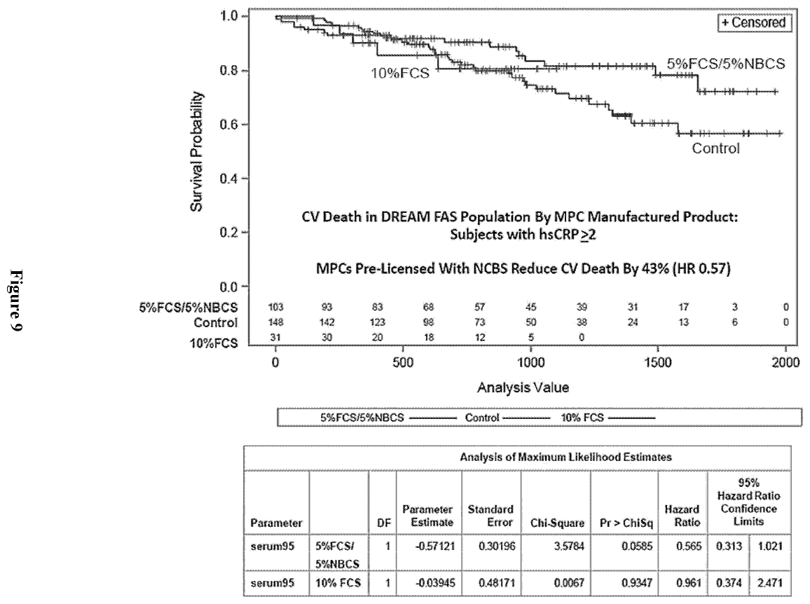

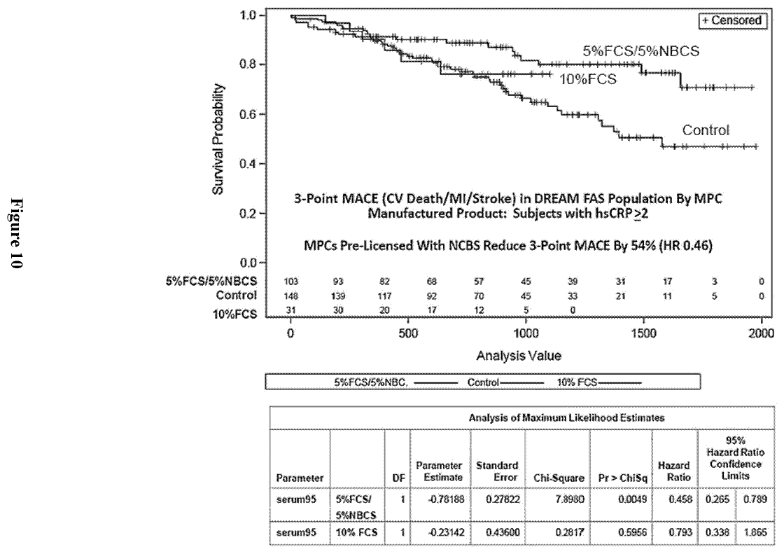

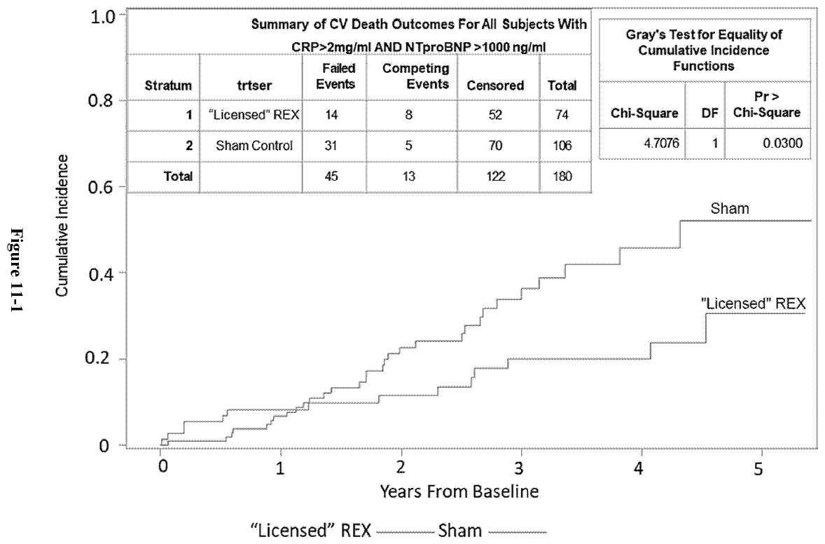

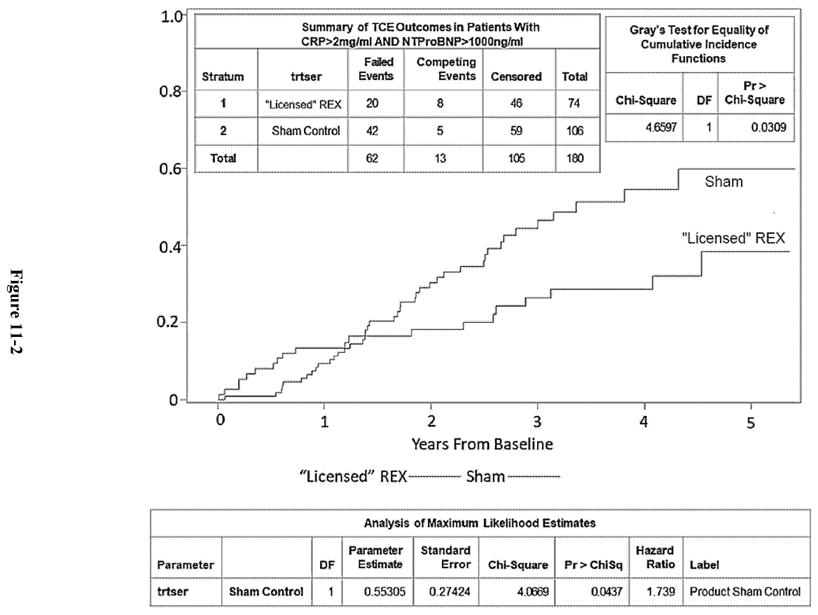

MPCs cultured in media supplemented with newborn serum reduced incidence of 3-point MACE (CV Death/MI/Stroke) in all patients (FIG. 8). MPCs cultured in media supplemented with newborn serum were also found to reduce other cardiac outcomes in HFrEF patients with CRP>2, including reducing the risk of cardiovascular death by 43% (FIG. 9) and incidence of 3-point MACE (CV Death/MI/Stroke) by 54% (FIG. 10). Notably, MPCs cultured in media supplemented with newborn calf serum (5% FCS/5% NBCS) significantly reduced CV death (FIG. 11A) and TCE (FIG. 11B) in the highest risk patients (CRP>2 mg/ml; NTpro-BNP>1000 ng/ml).

These data show that MPCs cultured in media supplemented with newborn serum provide improved therapeutic efficacy, in particular in the context of persistent inflammation.

Summary: MPCs expanded in media supplemented with newborn calf serum (NBCS) and/or pro-inflammatory cytokines:
improved left ventricular systolic dysfunction in HFrEF patients with inflammation, as measured by LS mean change in LVEF and LVESV at 12 months;
reduced cardiovascular death by 43% in high-risk NYHA Class II/III patients with HFrEF and inflammation; and,
reduced long-term 3 Point MACE by 54% in high-risk NYHA Class II/III patients with HFrEF and inflammation.

Taken together with the results in Examples 1 and 4, these human trial data indicate that supplementing cell culture media with newborn serum and/or pro-inflammatory cytokines provides a cell population with different functional characteristics, at least in terms of capacity to direct therapeutic efficacy in an inflammatory environment.

Without wishing to be bound by any particular theory, these data suggest that culturing in NBCS, in particular NBCS obtained from animals less than 20 days old, pre-licenses MPCs to respond more effectively to inflammatory environments and this capacity to respond is influenced (or associated with) increased Angiogenin levels. Indeed, Angiogenin levels may also represent a useful potency assay for therapeutic efficacy in the context of conditions associated with persistent inflammation such as heart failure. While the present inventors findings indicate pre-licensing using non-fetal serum, it is thought that one or more pro-inflammatory cytokines in the non-fetal serum facilitate pre-licensing. Accordingly, the present inventors findings are not limited to use of non-fetal serum but rather extend to culture expansion of MLPSCs in cell culture medium comprising at least one pro-inflammatory cytokine.

The findings of the present inventors underpin broad application of pre-licensed MPCs (e.g. MPCs cultured in cell culture medium comprising at least one pro-inflammatory cytokine; e.g. using culture medium comprising non-fetal serum, in particular newborn serum) for treatment of any disease or disorder wherein there is an elevated level of baseline inflammation, in particular diseases characterized by persistent inflammation such as heart failure.

### Example 7: Treatment of a HFrEF Patient Population with Licensed MPLSCs

Left ventricular assist devices (LVADs) were implanted in patients with end-stage heart failure with reduced ejection fraction (HFrEF). HFrEF patients who received LVADs were categorized as having either ischemic heart failure or non-ischemic heart failure. LVAD patients in the treatment arm received MPCs cultured in media supplemented with newborn serum or MPCs cultured in media containing 10% FBS. Control patients were not administered stem cell therapy.

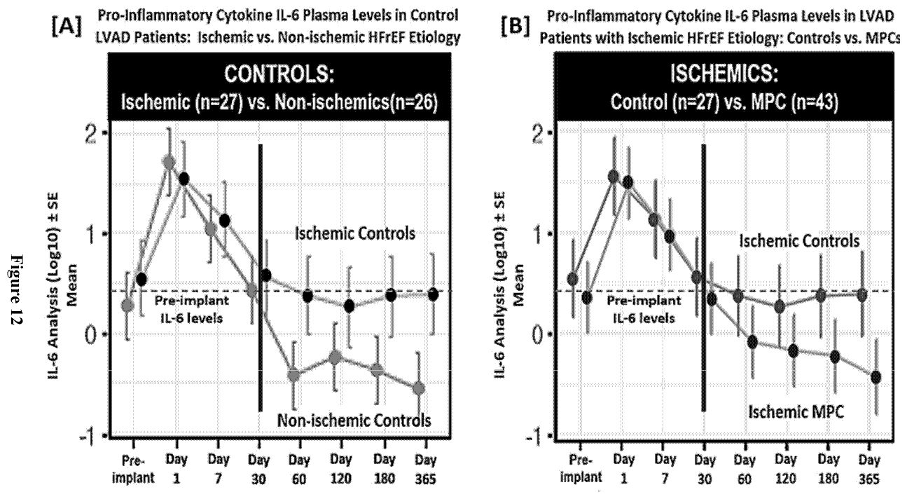

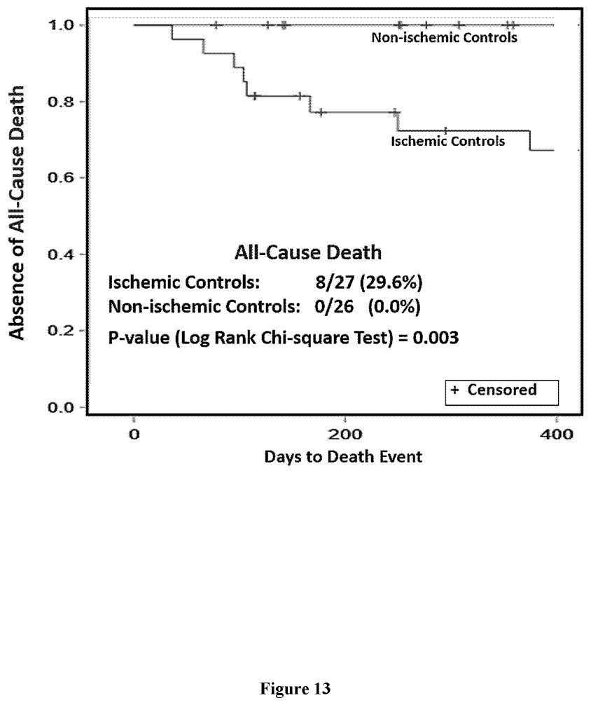

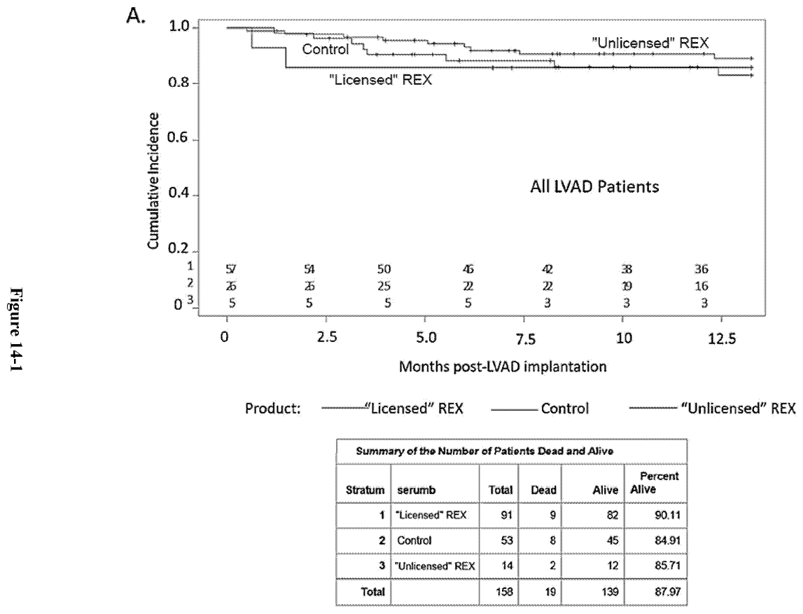

Inflammation was assessed based on serum IL-6 levels. Prior to LVAD implantation, both ischemic and non-ischemic heart failure control patients had similarly elevated IL-6 levels (FIG. 12). From Day 1 through Day 30 post-surgery, similar surgery-related IL-6 increases were observed in both control ischemic and non-ischemic groups (FIG. 12). From Day 30 through Day 365 post-surgery, IL-6 levels in the non-ischemic group reduced to levels below those observed pre-LVAD implantation. However, patients in the ischemic group maintained IL-6 levels similar to the level present pre-LVAD implantation and that were significantly higher than the non-ischemic group (FIG. 12). In addition, the ischemic control group were found to have significantly higher risk for all-cause death over 12 months after LVAD implantation, compared to non-ischemic controls (FIG. 13).

These data suggest that LVAD implantation reduces the inflammatory process associated with end-stage HFrEF in non-ischemic heart failure patients but not in ischemic heart failure patients. Furthermore, LVAD patients with ischemic end-stage HFrEF represent a specific patient subgroup who have persistent inflammation and are at high-risk of all-cause death.

FIG. 12B also shows that administration of MPCs cultured in media supplemented with newborn serum not only reduced IL-6 levels but that the IL-6 levels were, over time, reduced to levels corresponding with non-ischemic control.

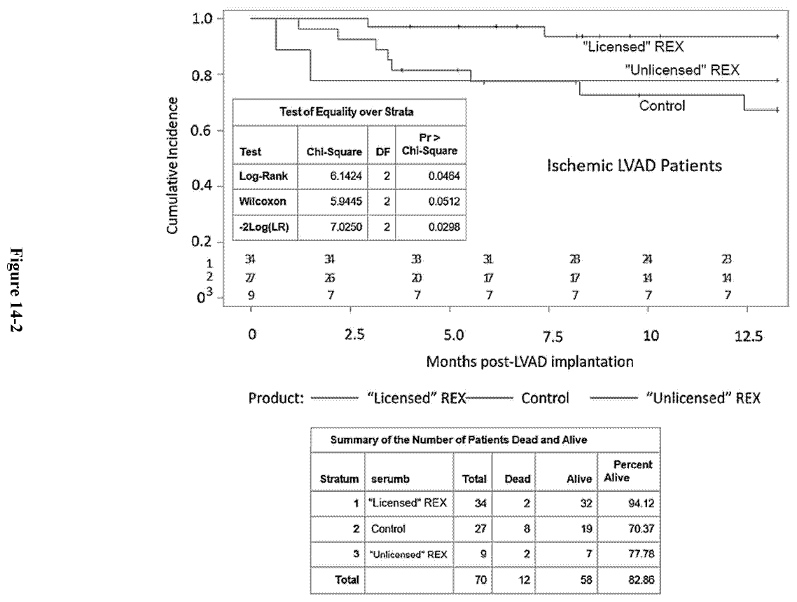

FIGS. 14 and 15 show all-cause death over a period of 12.5 months following LVAD implantation in patients who received MPCs cultured in media supplemented with newborn calf serum, MPCs cultured in media containing 10% FBS, and control patients who did not receive cell therapy. Surprisingly, ischemic LVAD patients who were administered the MPCs cultured in media supplemented with newborn calf serum had significantly reduced all-cause death compared to both the control group and patients who received unlicensed MPCs (FIG. 14B). Specifically, the MPCs cultured in media supplemented with newborn calf serum reduced all-cause death by 83% in the ischemic LVAD patients compared with the ischemic control group.

The findings of the present inventors provide additional evidence (on top of the studies in Example 6) that MPCs culture expanded in media supplemented with at least one pro-inflammatory cytokine and/or newborn calf serum are particularly effective in treating diseases characterized by persistent inflammation such as heart failure. They also provide basis for selecting a patient that will respond particularly well to treatment with pre-licensed MPCs, based on the patient's level of persistent inflammation (e.g. ischemic LVAD patients).

### Example 8: Media Analysis and Summary of Findings

Based on the data described in Example 1, cytokine levels were increased in culture medium used to expand MLPSC populations characterised by increase(s) in one or more angiogenic markers and increased therapeutic efficacy in heart failure patients. The correlation between increased pro-inflammatory cytokine levels in culture media and therapeutic efficacy in separate disease indications associated with inflammation suggests a pre-licensing effect on MLPSCs.

Surprisingly, MLPSCs described herein appear to have been pre-licensed by culture with pro-inflammatory cytokines, despite these cytokines being present at very low levels (e.g. pg/ml levels). This is surprising because it was not previously envisaged that pro-inflammatory cytokines, in particular TNF-alpha and IFN-gamma, could have such dramatic impacts (e.g. increased angiogenic potential; increased therapeutic efficacy in disease indications such as heart failure and GvHD) when present at pg/ml levels. Without wishing to be bound by any particular theory, the data provided by the present inventors, surprisingly suggest synergistic and/or more than additive effects of cytokines in the context of MLPSC culture expansion. For example, the present data indicates that provision of culture medium comprising TNF-alpha and IFN-gamma at concentrations <1 ng/ml can have profound impacts on MLPSCs culture expanded in the same and, that these impacts can be characterised based on levels of various angiogenic markers and/or clinical efficacy in patients.

Accordingly, the present inventors findings represent a significant advance in the art as they have shown how to prepare novel MLPSC populations that can direct improved therapeutic efficacy, in particular in the context of inflammation. These findings not only suggest that improved MLPSC populations can be provided through culture expansion in media supplemented with pro-inflammatory cytokines, they also indicate that relevant pro-inflammatory cytokines can be provided through culture expansion in medium supplemented with newborn serum. Accordingly, the present inventors findings underpin criteria for culture expansion of MLPSC in serum and serum free media.

### Example 9: MLPSC Isolation and Expansion

MLPSCs can be isolated using techniques such as STRO-3+ immunoselection of MPCs or density gradient separation of MSCs.

In general, relevant for bone marrow derived MLPSC, bone marrow (BM) is harvested from healthy normal adult volunteers (20-35 years old). Briefly, 40 ml of BM is aspirated from the posterior iliac crest into lithium-heparin anticoagulant-containing tubes.

BMMNC are prepared by density gradient separation using Lymphoprep (Nycomed Pharma, Oslo, Norway) as previously described (Zannettino et al. 1998). Following centrifugation at 400×g for 30 minutes at 4 C, the buffy layer is removed with a transfer pipette and washed three times in “HHF”, composed of Hank's balanced salt solution (HBSS; Life Technologies, Gaithersburg, MD), containing 5% fetal calf serum (FCS, CSL Limited, Victoria, Australia).

Relevant for immunoselection, STRO-3+ (or TNAP+) cells are subsequently isolated by magnetic activated cell sorting as previously described (Gronthos et al. 2003; Gronthos and Simmons 1995). Briefly, approximately 1-3×10⁸ BMMNC are incubated in blocking buffer, consisting of 10% (v/v) normal rabbit serum in HHF for 20 minutes on ice. The cells are incubated with 200 ul of a 10 ug/ml solution of STRO-3 mAb in blocking buffer for 1 hour on ice. The cells are subsequently washed twice in HHF by centrifugation at 400×g. A 1/50 dilution of goat anti-mouse-biotin (Southern Biotechnology Associates, Birmingham, UK) in HHF buffer is added and the cells incubated for 1 hour on ice. Cells are washed twice in MACS buffer (Ca2+- and Mn2+-free PBS supplemented with 1% BSA, 5 mM EDTA and 0.01% sodium azide) as above and resuspended in a final volume of 0.9 ml MACS buffer.

One hundred ul streptavidin microbeads (Miltenyi Biotec; Bergisch Gladbach, Germany) are added to the cell suspension and incubated on ice for 15 minutes. The cell suspension is washed twice and resuspended in 0.5 ml of MACS buffer and subsequently loaded onto a mini MACS column (MS Columns, Miltenyi Biotec), and washed three times with 0.5 ml MACS buffer to retrieve the cells which did not bind the STRO-3 mAb (deposited on 19 Dec. 2005 with American Type Culture Collection (ATCC) under accession number PTA-7282—see International Publication No. WO 2006/108229). After addition of a further 1 ml MACS buffer, the column is removed from the magnet and the TNAP+ cells are isolated by positive pressure. An aliquot of cells from each fraction can be stained with streptavidin-FITC and the purity assessed by flow cytometry.

Alternatively, MSCs may be expanded from BMMNC using plastic adherence techniques. For example, bone marrow mononuclear cells can be isolated using ficoll-hypaque and placed into two T175 flasks with 50 ml per flask of culture expansion medium which includes alpha modified MEM (αMEM) containing gentamycin, glutamine (2 mM) and 10% (v/v) fetal bovine serum (FBS).

Cells are cultured for 2-3 days in 37° C., 5% CO₂ at which time the non-adherent cells are removed; the remaining adherent cells are continually cultured until cell confluence reaches 70% or higher (7-10 days), and then the cells are trypsinized and replaced in six T175 flasks with expansion medium.

It will be appreciated by persons skilled in the art that numerous variations and/or modifications may be made to the disclosure as shown in the specific embodiments without departing from the spirit or scope of the disclosure as broadly described. The present embodiments are, therefore, to be considered in all respects as illustrative and not restrictive.

The present application claims priority from U.S. 63/386,878 filed 9 Dec. 2022, U.S. 63/386,876 filed 9 Dec. 2022, U.S. 63/486,792 filed 24 Feb. 2023, U.S. 63/507,009 filed 8 Jun. 2023, U.S. 63/507,013 filed 8 Jun. 2023 and U.S. 63/513,777 filed 14 Jul. 2023, the disclosures of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present disclosure. It is not to be taken as an admission that any or all of these matters form part of the prior art base or were common general knowledge in the field relevant to the present disclosure as it existed before the priority date of each claim of this application.

1. A method of treating progressive heart failure in a subject, the method comprising administering to the subject a composition comprising a population of culture expanded mesenchymal lineage precursor or stem cells (MLPSCs) or conditioned media obtained therefrom, wherein the subject has persistent inflammation and, wherein the MLPSCs have been culture expanded in a cell culture media comprising at least one pro-inflammatory cytokine.

2. The method according to claim 1, wherein the MLPSCs have been culture expanded in media containing:
IFN-gamma and/or TNF-alpha; and/or,
one or more pro-inflammatory cytokines selected from the group consisting of IL-6; IL-8; IL-17A; MCP-1; MIP-1-alpha; MIP-1-beta; IP-10.

3. The method according to claim 1 or 2, wherein the media contains three or more pro-inflammatory cytokines.

4. The method according to claim 2 or 3, wherein the media contains two or more pro-inflammatory cytokines selected from the group consisting of IL-6; IL-8; IL-17A; MCP-1; MIP-1-alpha; MIP-1-beta; IP-10.

5. The method according to any one of claims 1 to 4, wherein the media contains IL-6.

6. The method according to any one of claims 1 to 5, wherein the media contains IL-8 and/or IL-17A.

7. The method according to any one of claims 1 to 6, wherein the media contains IFN-gamma and TNF-alpha.

8. The method according to any one of claims 2 to 7, wherein the level of IFN-gamma is <1 ng/ml, preferably <500 pg/ml, more preferably <100 pg/ml.

9. The method according to any one of claims 2 to 8, wherein the level of TNF-alpha is <1 ng/ml, preferably <750 pg/ml, more preferably <400 pg/ml.

10. The method according to any one of claims 1 to 9, wherein the media contains serum which comprises the pro-inflammatory cytokine(s).

11. The method according to claim 10, wherein the media comprises a non-fetal serum.

12. The method according to claim 10 or 11, wherein the serum is a newborn mammalian serum.

13. The method according to any one of claims 10 to 12, wherein the serum is newborn calf serum (NBCS).

14. The method according to any one of claims 10 to 13, wherein the serum is obtained no more than 21 days after birth.

15. The method according to any one of claims 10 to 14, wherein the serum is obtained between the day of birth and 21 days after birth; between the day of birth and 14 days after birth; between the day of birth and 10 days after birth; or, between the day of birth and 7 days after birth.

16. The method according to any one of claims 10 to 15, wherein the serum is obtained between the day of birth and 10 days after birth.

17. The method according to any one of claims 1 to 16, wherein the media is characterised by one or more or all of the following:
i. a level of IFN-gamma greater than 1 pg/ml;
ii. a level of TNF-alpha greater than 2 pg/ml;
iii. a level of IL-6 greater than 3 pg/ml;
iv. a level of IL-8 greater than 500 pg/ml;
v. a level of IL-17A greater than 0.2 pg/ml;
vi. a level of MCP-1 greater than 3 pg/ml;
vii. a level of MIP-1-alpha greater than 0.5 pg/ml;
viii. a level of MIP-1-beta greater than 3 pg/ml;
ix. a level of IP-10 greater than 500 pg/ml.

18. The method according to any one of claims 10 to 17, wherein the media comprises at least 5% (v/v) newborn calf serum.

19. The method according to any one of claim 1 to 9 or 17, wherein the media is serum free and/or xeno free.

20. The method according to any one of claims 10 to 19, wherein the media comprises 5% non-fetal serum.

21. The method according to any one of claims 10 to 19, wherein the media comprises 5% non-fetal serum and 5% fetal serum.

22. The method according to claim 20 or 21, wherein the non-fetal serum is NBCS.

23. The method according to any one of claim 1 to 9 or 17, wherein the media is a xeno-free media.

24. The method according to claim 23, wherein the xeno-free media comprises human serum.

25. The method according to any one of claim 1 to 9 or 17, wherein the media is serum-free.

26. The method according to claim any one of claims 1 to 25, wherein the subject has myocardial ischemia and/or diabetes.

27. The method according to any one of claims 1 to 25, wherein the subject's CRP level is ≥2 mg/L.

28. The method according to any one of claims 1 to 27, wherein the subject has a LVEF of less than about 45%, preferably less than 40%, preferably between 30 and 35%.

29. The method according to any one of claims 1 to 28, wherein the subject has a LVESV greater than 70 ml.

30. The method according to any one of claims 1 to 29, wherein the subject has a LVESV between 70 ml and 160 ml.

31. The method according to any one of claims 1 to 30, wherein the subject has Class II heart failure according to the New York Heart Association (NYHA) classification scale.

32. The method according to any one of claims 1 to 31, which comprises the steps of: i) selecting a subject having a micro-vascular disease and/or a macro-vascular disease for treatment, and ii) administering the MLPSCs.

33. The method according to any one of claims 1 to 32, which comprises the steps of: i) selecting a subject having a CRP level ≥2 mg/L for treatment, and ii) administering to the MLPSCs.

34. The method according to any one of claims 1 to 33, wherein the subject's level of N-terminal pro-B-type natriuretic peptide (NT-proBNP) is:
>1000 μg/mL, or,
between 1000 pg/ml and 2500 pg/ml.

35. The method according to any one of claims 1 to 34, wherein the subject's C-reactive protein (CRP) level is between 2 and 5 mg/L, preferably between 2 and 4 mg/L, preferably between 2 and 3 mg/L.

36. The method according to any one of claims 1 to 35, wherein the subject has had a heart failure hospitalisation event over the previous 9 months.

37. The method according to any one of claims 1 to 36, wherein the subject has persistent left ventricular dysfunction.

38. The method according to any one of claims 1 to 37, wherein the subject's heart failure results from an ischaemic event or from a non-ischaemic event.

39. The method according to any one of claims 1 to 38, wherein the subject has a reduced risk of cardiac death after treatment.

40. The method according to any one of claims 1 to 39, wherein treatment improves the subject's LVEF by at least 4 percentage points.

41. The method according to any one of claims 1 to 40, wherein treatment improves the subject's LVEF by at least 5 percentage points or at least 6 percentage points.

42. The method according to any one of claims 1 to 41, wherein treatment improves the subject's LVEF by between 4 and 7 percentage points; between 5 and 7 percentage points.

43. The method according to any one of claims 1 to 42, wherein treatment:
improves the subject's LVESV by at least 17 ml;
improves the subject's LVESV by at least 20 ml;
improves the subject's LVESV by between 15 ml and 30 ml; or
improves the subject's LVEDV by at least 15 ml; preferably between 15 ml and 25 ml.

44. The method according to claim 43, wherein the reduced risk is relative to risk of cardiac death in a subject that has not been administered MLPSCs.

45. The method according to any one of claims 1 to 44, wherein the subject has a reduced risk of ischaemic MACE (MI or stroke) after treatment.

46. The method according to any one of claims 1 to 45, wherein the subject has a left ventricular assist device (LVAD).

47. The method according to claim 46, wherein the subject's IL-6 level is increased relative to baseline at least 60 days after LVAD implantation.

48. The method according to claim 46 or 47, wherein treatment reduces the subject's risk of all-cause death.

49. The method according to any one of claims 46 to 48, wherein treatment reduces the subject's risk of all-cause death by between 10% and 90%; greater than 50%; between 20 and 85%; preferably about 80%.

50. The method according to any one of claims 1 to 49, wherein the composition is administered transendocardially and/or intravenously.

51. The method according to any one of claims 1 to 50, wherein the MLPSCs are mesenchymal precursor cells (MPCs).

52. The method according to claim 51, wherein the MPCs are isolated from bone mononuclear cells with an anti-STRO-3 antibody before culture expansion.

53. The method according to any one of claims 1 to 52, wherein the MLPSCs are mesenchymal stem cells (MSCs).

54. The method according to any one of claims 1 to 53, wherein the cells are allogeneic.

55. The method according to any one of claims 1 to 54, wherein the cells have been cryopreserved prior to administration.

56. The method according to any one of claims 1 to 55 which comprises administering between 1×10⁷ and 2×10⁸ cells.

57. The method according to any one of claims 1 to 56, wherein the composition further comprises Plasma-Lyte A, dimethyl sulfoxide (DMSO), human serum albumin (HSA).

58. The method according to any one of claims 1 to 57, wherein the composition further comprises Plasma-Lyte A (70%), DMSO (10%), HSA (25%) solution, the HSA solution comprising 5% HSA and 15% buffer.

59. The method according to any one of claims 1 to 58, wherein the composition comprises greater than 6.68×10⁶ viable cells/mL.

60. The method according to any one of claims 1 to 59, wherein the composition comprises human bone marrow-derived allogeneic MPCs isolated from bone mononuclear cells with anti-STRO-3 antibodies, expanded ex vivo in culture media comprising NBCS, and cryopreserved.

61. A method for selecting a population of culture expanded mesenchymal lineage precursor or stem cells (MLPSCs) for use in treatment of progressive heart failure in a subject wherein the MLPSCs have been culture expanded in a cell culture media comprising at least one pro-inflammatory cytokine, the method comprising:
(i) obtaining a population of MLPSCs,
(ii) determining the level of one or more angiogenic markers in the population of MLPSCs, wherein the one or more angiogenic markers is selected from the group consisting of:
the level of VEGF, angiogenin, SDF-1α expressed by the MLPSCs under culture conditions; and/or,
the level of endothelial network formation, endothelial network length, endothelial branch length measured after treating a population of endothelial cells with conditioned media obtained from the MLPSCs;
(iii) selecting for use in treatment the MLPSCs that have increased level(s) of the one or more angiogenic markers.

62. A culture expanded population of mesenchymal lineage precursor or stem cells (MLPSCs), wherein the population of MLPSCs are selected based on high angiogenic potential as determined by the level of angiogenin expressed by the MLPSCs under culture conditions.

63. A culture expanded population of mesenchymal lineage precursor or stem cells (MLPSCs), wherein the population of MLPSCs are selected based on high angiogenic potential as determined by the level of one or more of the following measured after treating a population of endothelial cells with conditioned media obtained from the MLPSCs:
endothelial network formation;
endothelial network length; or,
endothelial branch length.

64. The culture expanded population of claim 62, wherein MLPSCs that express an increased level of angiogenin relative to a control population are selected.

65. The culture expanded population of claim 63, wherein MLPSCs that induce increased levels of one or more of endothelial network formation, endothelial length, or endothelial branch length, relative to a control population are selected.

66. The culture expanded population according to claim 64 or 65, wherein the control population is a population of MLPSCs that have been culture expanded in a cell culture media comprising 10% fetal calf serum.

67. The culture expanded population according to any one of claims 62 to 66, wherein MLPSCs characterised by one or more of the following are selected:
express a level of angiogenin greater than about 1200 pg/ml;
induce endothelial network formation greater than about 0.12 mm²/mm²;
induce endothelial network length greater than about 5 mm²/mm²;
induce endothelial branch length greater than about 15 l/mm².

68. A method of manufacturing drug product which comprises a population of mesenchymal lineage precursor or stem cells (MLPSCs), the method comprising: acquiring a determination of whether a test population of MLPSCs have a predetermined level of angiogenic potential under culture conditions, and processing at least a portion of the test population of MLPSCs as a drug product if the test population of MLPSCs have at least the predetermined level of angiogenic potential under culture conditions, thereby manufacturing the drug product; or discarding at least a portion of the test population of MLPSCs if the population of MLPSCs has less than the predetermined level of angiogenic potential under culture conditions, wherein angiogenic potential is measured by a predetermined level of angiogenin measured under culture conditions.

69. A method of manufacturing drug product which comprises a population of mesenchymal lineage precursor or stem cells (MLPSCs), the method comprising: acquiring a determination of whether a test population of MLPSCs have a predetermined level of angiogenic potential under culture conditions, and processing at least a portion of the test population of MLPSCs as a drug product if the test population of MLPSCs have at least the predetermined level of angiogenic potential under culture conditions, thereby manufacturing the drug product; or discarding at least a portion of the test population of MLPSCs if the population of MLPSCs has less than the predetermined level of angiogenic potential under culture conditions, wherein angiogenic potential is measured by:
a predetermined level of one or more of the following as measured after treating a population of endothelial cells with conditioned media obtained from the MLPSCs:
endothelial network formation;
endothelial network length; and/or,
endothelial branch length measured in an in-vitro angiogenesis assay.

70. The method according to claim 68, wherein the predetermined level of angiogenin is measured in conditioned media obtained from the test population of MLPSCs.

71. The method according to claim 69, wherein the predetermined level is:
endothelial network formation is greater than about 0.12 mm²/mm²;
endothelial network length is greater than about 5 mm²/mm²;
endothelial branch length is greater than about 15 l/mm².

72. The culture expanded population according to any one of claims 62 to 67, or the method of any one claims 68 to 71, wherein the MLPSCs have been culture expanded in cell culture media containing:
IFN-gamma and/or TNF-alpha; and/or,
one or more pro-inflammatory cytokines selected from the group consisting of IL-6; IL-8; IL-17A; MCP-1; MIP-1-alpha; MIP-1-beta; IP-10.

73. The culture expanded population or method according to any one of claims 62 to 71, wherein the media contains serum which comprises the pro-inflammatory cytokines.

74. The culture expanded population or method according to claim 73, wherein the serum is a newborn calf serum (NBCS).

75. The culture expanded population or method according to claim 74, wherein the serum is obtained no more than 21 days after birth.

76. A method for determining the potency of a population of culture expanded mesenchymal lineage precursor or stem cells (MLPSCs) wherein the MLPSCs have been culture expanded in a cell culture media comprising at least one pro-inflammatory cytokine, the method comprising determining the level of one or more angiogenic markers in the population of MLPSCs, wherein the one or more angiogenic markers is selected from the group consisting of:
the level of VEGF, angiogenin, SDF-1α expressed by the MLPSCs under culture conditions; and/or,
the level of endothelial network formation, endothelial network length, endothelial branch length measured after treating a population of endothelial cells with conditioned media obtained from the MLPSCs,
wherein an increased level of one or more angiogenic markers is indicative of biological activity or therapeutic efficacy.

77. The method according to claim 76, wherein the cell culture media comprises a non-fetal serum, preferably newborn calf serum (NCBS).
